SEQUENCE LISTING (59 sequences)

SEQ ID NO: 1 - DNA, 1677 bp, Pseudomonas putida
atgtacgactatataatcgttggtgctggatctgcaggatgtgtgcttgctaatcgtctttcggccgacccctctaaaagagtttgtttacttgaagctgggccgcgagatacgaatccgctaattcatatgccgttaggtattgctttgctttcaaatagtaaaaagttgaattgggcttttcaaactgcgccacagcaaaatctcaacggccggagccttttctggccacgaggaaaaacgttaggtggttcaagctcaatcaacgcaatggtctatatccgagggcatgaagacgattaccacgcatgggagcaggcggccggccgctactggggttggtaccgggctcttgagttgttcaaaaggcttgaatgcaaccagcgattcgataagtccgagcaccatggggttgacggagaattagctgttagtgatttaaaatatatcaatccgcttagcaaagcattcgtgcaagccggcatggaggccaatattaatttcaacggagatttcaacggcgagtaccaggacggcgtagggttctatcaagtaacccaaaaaaatggacaacgctggagctcggcgcgtgcattcttgcacggtgtactttccagaccaaatctagacatcattactgatgcgcatgcatcaaaaattctttttgaagaccgtaaggcggttggtgtttcttatataaagaaaaatatgcaccatcaagtcaagacaacgagtggtggtgaagtacttcttagtcttggcgcagtcggcacgcctcaccttctaatgctttctggtgttggggctgcagccgagcttaaggaacatggtgtttctctagtccatgatcttcctgaggtggggaaaaatcttcaagatcatttggacatcacattgatgtgcgcagcaaattcgagagagccgataggtgttgctctttctttcatccctcgtggtgtctcgggtttgttttcatatgtgtttaagcgcgaggggtttctcactagtaacgtggcagagtcgggtggttttgtaaaaagttctcctgatcgtgatcggcccaatttgcagtttcatttccttccaacttatcttaaagatcacggtcgaaaaatagcgggtggttatggttatacgctacatatatgtgatcttttgcctaagagccgaggcagaattggcctaaaaagcgccaatccattacagccgcctttaattgacccgaactatcttagcgatcatgaagatattaaaaccatgattgcgggtattaagatagggcgcgctattttgcaggccccatcgatggcgaagcattttaagcatgaagtagtaccgggccaggctgttaaaactgatgatgaaataatcgaagatattcgtaggcgagctgagactatataccatccggtaggtacttgtaggatgggtaaagatccagcgtcagttgttgatccgtgcctgaagatccgtgggttggcaaatattagagtcgttgatgcgtcaattatgccgcacttggtcgcgggtaacacaaacgctccaactattatgattgcagaaaatgcggcagaaataattatgcggaatcttgatgtggaagcattagaggctagcgctgagtttgctcgcgagggtgcagagctagagttggccatgatagctgtctgcatgtaa

SEQ ID NO: 2 - protein, 557 aa, Pseudomonas putida
MYDYIIVGAGSAGCVLANRLSADPSKRVCLLEAGPRDTNPLIHMPLGIALLSNSKKLNWAFQTAPQQNLNGRSLFWPRGKTLGGSSSINAMVYIRGHEDDYHAWEQAAGRYWGWYRALELFKRLECNQRFDKSEHHGVDGELAVSDLKYINPLSAFVQAGMEANINFNGDFNGEYQDGVGFYQVTQKNGQRWSSARAFLHGVLSRPNLDIITDAHASKILFEDRKAVGVSYIKKNMHHQVKTTSGGEVLLSLGAVGTPHLLMLSGVGAAAELKEHGVSLVHDLPEVGKNLQDHLDITLMCAANSREPIGVALSFIPRGVSGLFSYVFKREGFLTSNVAESGGFVKSSPDRDRPNLQFHFLPTYLKDHGRKIAGGYGYTLHICDLLPKSRGRIGLKSANPLQPPLIDPNYLSDHEDIKTMIAGIKIGRAILQAPSMAKHFKHEVVPGQAVKTDDEIIEDIRRRAETIYHPVGTCRMGKDPASVVDPCLKIRGLANIRVVDASIMPHLVAGNTNAPTIMIAENAAEIIMRNLDVEALEASAEFAREGAELELAMIAVCM

SEQ ID NO: 3 - DNA, 39 bp, Artificial Sequence
  forward primer
actgggatccgatgtacgactatataatcgttggtgctg

SEQ ID NO: 4 - DNA, 35 bp, Artificial Sequence
  reverse primer
actgagatctttacatgcagacagctatcatggcc

SEQ ID NO: 5 - DNA, 1380 bp, Chromobacterium violaceum
atgcaaaaacaacgcaccacctcacaatggcgcgaactggatgccgcacaccacctgcacccgtttaccgacaccgcaagcctgaatcaggccggcgcccgtgttatgacccgcggcgaaggtgtgtatctgtgggattctgagggtaacaaaattatcgacggcatggctggtctgtggtgcgttaatgtcggctatggtcgtaaagattttgccgaagcggcccgtcgccaaatggaagaactgccgttctacaacacctttttcaaaaccacgcatccggcggtggttgaactgagcagcctgctggcggaagttacgccggccggctttgatcgtgtgttctataccaattcaggttcggaaagcgtggatacgatgatccgcatggttcgtcgctactgggacgtccagggcaaaccggaaaagaaaaccctgatcggtcgttggaacggctatcatggttctacgattggcggtgcaagtctgggcggtatgaaatacatgcacgaacagggcgatctgccgattccgggtatggcgcatatcgaacaaccgtggtggtacaaacacggcaaagatatgaccccggacgaatttggtgtcgtggcagctcgctggctggaagaaaaaattctggaaatcggcgccgataaagtggcggcctttgttggcgaaccgattcagggtgcgggcggtgtgattgttccgccggccacctattggccggaaattgaacgtatctgccgcaaatacgatgttctgctggtcgcagacgaagttatttgtggctttggtcgtaccggcgaatggttcggtcatcagcactttggcttccaaccggacctgtttacggcagctaaaggcctgagttccggttatctgccgatcggcgccgtcttcgtgggtaaacgcgttgcagaaggtctgattgctggcggtgattttaatcatggcttcacctatagcggtcacccggtctgtgcggccgtggcacatgctaatgtggcagctctgcgtgacgaaggcatcgtgcagcgcgttaaagatgacattggtccgtatatgcaaaaacgttggcgcgaaacgtttagccgtttcgaacacgtcgatgacgtgcgcggcgttggtatggtccaggcatttaccctggtgaaaaataaagctaaacgcgaactgtttccggatttcggcgaaattggtacgctgtgccgtgacatctttttccgcaacaatctgattatgcgtgcgtgtggtgatcacattgttagcgccccgccgctggttatgacccgcgcagaagtcgacgaaatgctggccgtggcggaacgctgcctggaagaatttgaacagaccctgaaagctcgtggcctggcgtaa

SEQ ID NO: 6 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 7 - DNA, 47 bp, Artificial Sequence
  forward primer
cgagatcttaaggagatatataatgcaaaaacaacgcaccacctcac

SEQ ID NO: 8 - DNA, 40 bp, Artificial Sequence
  reverse primer
actgctcgaggaattcttacgccaggccacgagctttcag

SEQ ID NO: 9 - DNA, 1137 bp, Bacillus subtilis
atgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgacttaaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

SEQ ID NO: 10 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 11 - DNA, 50 bp, Artificial Sequence
  forward primer
actggaattctaaggagatatataatgatcataggggttcctaaagagat

SEQ ID NO: 12 - DNA, 35 bp, Artificial Sequence
  reverse primer
actgctcgagttaagcacccgccacagatgattca

SEQ ID NO: 13 - DNA, 39 bp, Artificial Sequence; identical to SEQ ID NO: 3
  forward cloning primer

SEQ ID NO: 14 - DNA, 35 bp, Artificial Sequence; identical to SEQ ID NO: 12
  reverse cloning primer

SEQ ID NO: 15 - DNA, 1134 bp, Artificial Sequence
  codon optimised for E coli expression
atgcgtgaaccgctgacgctggatgatttcgctcgcctggcccgtggtcaactgccggcagcaacctgggattttatcgcgggtggcgcaggtcgtgaacgtaccctggcagcaaacgaagctgtgtttggtgcagttcgtctgcgcccgcgtgcgctgccgggcattgaagaaccggataccagcgtggaagttctgggttctcgctggccggcaccggttggtatcgctccggtcgcgtatcatggtctggcacacccggatggtgaaccggcaacggcagctgcggccggcgctctgggtctgccgctggttgtgagtacctttgcaggccgctccctggaagaagtggcccgtgcagcttcagcaccgctgtggctgcaactgtattgcttccgcgatcacgaaaccacgctgggtctggcacgtcgcgcacgtgactcgggctaccaagcgctggttctgaccgtcgatacgccgtttaccggtcgtcgcctgcgcgacctgcgtaacggcttcgctgtgccggcgcatattacgccggcgaatctgaccggcaccgcggcggcgggcagcgcaaccccgggcgcacactcgcgcctggcgtttgatcgtcgcctggactggagcttcgttgcacgtctgggcgctgcatctggtctgccggtgctggcaaaaggtgttctgaccgcaccggatgcagaagccgcagtcgctgcgggcgtggcaggtatcgtcgtgagtaatcatggcggtcgccagctggacggtgcgccggcaacgctggaagcgctgccggaagttgtctccgccgtccgcggtcgttgtccggtgctgctggatggcggtgttcgtaccggtgcagacgtcctggcagcactggctctgggtgctcgcgcggtcctggtgggccgtccggcactgtacgccctggcagtgggcggtgcaagtggtgttcgtcgcatgctgacgctgctgaccgaagatttcgcggacacgatggttctgaccggtcacgctgcaaccggcacgattggtccggatacgctggctccgccgcatcacgcaccgccgcatcacggtccgccgaccgctccgcgtccggcaccgcatcgcgatcgttctcacggctaa

SEQ ID NO: 16 - protein, 377 aa, Artificial Sequence
  codon optimised for E coli
MREPLTLDDFARLARGQLPAATWDFIAGGAGRERTLAANEAVFGAVRLRPRALPGIEEPDTSVEVLGSRWPAPVGIAPVAYHGLAHPDGEPATAAAAGALGLPLVVSTFAGRSLEEVARAASAPLWLQLYCFRDHETTLGLARRARDSGYQALVLTVDTPFTGRRLRDLRNGFAVPAHITPANLTGTAAAGSATPGAHSRLAFDRRLDWSFVARLGAASGLPVLAKGVLTAPDAEAAVAAGVAGIVVSNHGGRQLDGAPATLEALPEVVSAVRGRCPVLLDGGVRTGADVLAALALGARAVLVGRPALYALAVGGASGVRRMLTLLTEDFADTMVLTGHAATGTIGPDTLAPPHHAPPHHGPPTAPRPAPHRDRSHG

SEQ ID NO: 17 - DNA, 34 bp, Artificial Sequence
  forward primer
actgtcatgatgcgtgaaccgctgacgctggatg

SEQ ID NO: 18 - DNA, 35 bp, Artificial Sequence
  reverse primer
actggaattcttagccgtgagaacgatcgcgatgc

SEQ ID NO: 19 - DNA, 930 bp, Escherichia coli
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 20 - protein, 309 aa, Escherichia coli
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQ

SEQ ID NO: 21 - DNA, 49 bp, Artificial Sequence
  forward primer
actggaattctaaggagatatataatgaccacgaagaaagctgattaca

SEQ ID NO: 22 - DNA, 38 bp, Artificial Sequence
  reverse primer
actgagatctttattgattaacttgatctaaccagccc

SEQ ID NO: 23 - DNA, 1344 bp, Escherichia coli
atggatcagacatattctctggagtcattcctcaaccatgtccaaaagcgcgacccgaatcaaaccgagttcgcgcaagccgttcgtgaagtaatgaccacactctggccttttcttgaacaaaatccaaaatatcgccagatgtcattactggagcgtctggttgaaccggagcgcgtgatccagtttcgcgtggtatgggttgatgatcgcaaccagatacaggtcaaccgtgcatggcgtgtgcagttcagctctgccatcggcccgtacaaaggcggtatgcgcttccatccgtcagttaacctttccattctcaaattcctcggctttgaacaaaccttcaaaaatgccctgactactctgccgatgggcggtggtaaaggcggcagcgatttcgatccgaaaggaaaaagcgaaggtgaagtgatgcgtttttgccaggcgctgatgactgaactgtatcgccacctgggcgcggataccgacgttccggcaggtgatatcggggttggtggtcgtgaagtcggctttatggcggggatgatgaaaaagctctccaacaataccgcctgcgtcttcaccggtaagggcctttcatttggcggcagtcttattcgcccggaagctaccggctacggtctggtttatttcacagaagcaatgctaaaacgccacggtatgggttttgaagggatgcgcgtttccgtttctggctccggcaacgtcgcccagtacgctatcgaaaaagcgatggaatttggtgctcgtgtgatcactgcgtcagactccagcggcactgtagttgatgaaagcggattcacgaaagagaaactggcacgtcttatcgaaatcaaagccagccgcgatggtcgagtggcagattacgccaaagaatttggtctggtctatctcgaaggccaacagccgtggtctctaccggttgatatcgccctgccttgcgccacccagaatgaactggatgttgacgccgcgcatcagcttatcgctaatggcgttaaagccgtcgccgaaggggcaaatatgccgaccaccatcgaagcgactgaactgttccagcaggcaggcgtactatttgcaccgggtaaagcggctaatgctggtggcgtcgctacatcgggcctggaaatggcacaaaacgctgcgcgcctgggctggaaagccgagaaagttgacgcacgtttgcatcacatcatgctggatatccaccatgcctgtgttgagcatggtggtgaaggtgagcaaaccaactacgtgcagggcgcgaacattgccggttttgtgaaggttgccgatgcgatgctggcgcagggtgtgatttaa

SEQ ID NO: 24 - protein, 447 aa, Escherichia coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 25 - DNA, 50 bp, Artificial Sequence
  forward primer
actgagatcttaaggagatatataatggatcagacatattctctggagtc

SEQ ID NO: 26 - DNA, 35 bp, Artificial Sequence
  reverse primer
actgggtaccttaaatcacaccctgcgccagcatc

SEQ ID NO: 27 - DNA, 2262 bp, Escherichia coli
atgtcgcaacataacgaaaagaacccacatcagcaccagtcaccactacacgattccagcgaagcgaaaccggggatggactcactggcacctgaggacggctctcatcgtccagcggctgaaccaacaccgccaggtgcacaacctaccgccccagggagcctgaaagcccctgatacgcgtaacgaaaaacttaattctctggaagacgtacgcaaaggcagtgaaaattatgcgctgaccactaatcagggcgtgcgcatcgccgacgatcaaaactcactgcgtgccggtagccgtggtccaacgctgctggaagattttattctgcgcgagaaaatcacccactttgaccatgagcgcattccggaacgtattgttcatgcacgcggatcagccgctcacggttatttccagccatataaaagcttaagcgatattaccaaagcggatttcctctcagatccgaacaaaatcaccccagtatttgtacgtttctctaccgttcagggtggtgctggctctgctgataccgtgcgtgatatccgtggctttgccaccaagttctataccgaagagggtatttttgacctcgttggcaataacacgccaatcttctttatccaggatgcgcataaattccccgattttgttcatgcggtaaaaccagaaccgcactgggcaattccacaagggcaaagtgcccacgatactttctgggattatgtttctctgcaacctgaaactctgcacaacgtgatgtgggcgatgtcggatcgcggcatcccccgcagttaccgcaccatggaaggcttcggtattcacaccttccgcctgattaatgccgaagggaaggcaacgtttgtacgtttccactggaaaccactggcaggtaaagcctcactcgtttgggatgaagcacaaaaactcaccggacgtgacccggacttccaccgccgcgagttgtgggaagccattgaagcaggcgattttccggaatacgaactgggcttccagttgattcctgaagaagatgaattcaagttcgacttcgatcttctcgatccaaccaaacttatcccggaagaactggtgcccgttcagcgtgtcggcaaaatggtgctcaatcgcaacccggataacttctttgctgaaaacgaacaggcggctttccatcctgggcatatcgtgccgggactggacttcaccaacgatccgctgttgcagggacgtttgttctcctataccgatacacaaatcagtcgtcttggtgggccgaatttccatgagattccgattaaccgtccgacctgcccttaccataatttccagcgtgacggcatgcatcgcatggggatcgacactaacccggcgaattacgaaccgaactcgattaacgataactggccgcgcgaaacaccgccggggccgaaacgcggcggttttgaatcataccaggagcgcgtggaaggcaataaagttcgcgagcgcagcccatcgtttggcgaatattattcccatccgcgtctgttctggctaagtcagacgccatttgagcagcgccatattgtcgatggtttcagttttgagttaagcaaagtcgttcgtccgtatattcgtgagcgcgttgttgaccagctggcgcatattgatctcactctggcccaggcggtggcgaaaaatctcggtatcgaactgactgacgaccagctgaatatcaccccacctccggacgtcaacggtctgaaaaaggatccatccttaagtttgtacgccattcctgacggtgatgtgaaaggtcgcgtggtagcgattttacttaatgatgaagtgagatcggcagaccttctggccattctcaaggcgctgaaggccaaaggcgttcatgccaaactgctctactcccgaatgggtgaagtgactgcggatgacggtacggtgttgcctatagccgctacctttgccggtgcaccttcgctgacggtcgatgcggtcattgtcccttgcggcaatatcgcggatatcgctgacaacggcgatgccaactactacctgatggaagcctacaaacaccttaaaccgattgcgctggcgggtgacgcgcgcaagtttaaagcaacaatcaagatcgctgaccagggtgaagaagggattgtggaagctgacagcgctgacggtagttttatggatgaactgctaacgctgatggcagcacaccgcgtgtggtcacgcattcctaagattgacaaaattcctgcctga

SEQ ID NO: 28 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 29 - DNA, 49 bp, Artificial Sequence
  forward primer
actgggtacctaaggagatatataatgtcgcaacataacgaaaagaacc

SEQ ID NO: 30 - DNA, 36 bp, Artificial Sequence
  reverse primer
actgctcgagtcaggcaggaattttgtcaatcttag

SEQ ID NO: 31 - DNA, 34 bp, Artificial Sequence; identical to SEQ ID NO: 17
  forward primer

SEQ ID NO: 32 - DNA, 36 bp, Artificial Sequence; identical to SEQ ID NO: 30
  reverse primer

SEQ ID NO: 33 - DNA, 1248 bp, Solanum tuberosum
atgaaaaagcgtatcggtattgttggtgcaggcactgccggcctccatcttggtctcttccttcgtcagcatgacgtcgacgtcactgtgtacactgatcgtaagcccgatgagtacagcggactgcgtctcctgaataccgttgctcacaacgcggtgacggtgcagcgggaggttgccctcgacgtcaatgagtggccgtctgaggagtttggttatttcggccactactactacgtaggtgggccgcagcccatgcgtttctacggtgatctcaaggctcccagccgtgcagtggactaccgtctctaccagccgatgctgatgcgtgcactggaagccaggggcggcaagttctgctacgacgcggtgtctgccgaagatctggaagggctgtcggagcagtacgatctgctggttgtgtgcactggtaaatacgccctcggcaaggtgttcgagaagcagtccgaaaactcgcccttcgagaagccgcaacgggcactgtgcgttggtctcttcaagggcatcaaggaagcaccgattcgcgcggtgactatgtccttctcgccagggcatggcgagctgattgagattccaaccctgtcgttcaatggcatgagcacagcgctggtgctcgaaaaccatattggtagcgatctggaagttctcgcccacaccaagtatgacgatgacccgcgtgcgttcctcgatctgatgctggagaagctgggtaagcatcatccttccgttgccgagcgcatcgatccggctgagttcgaccttgccaacagttctctggacatcctccagggtggtgttgtgccggcattccgcgacggtcatgcgaccctcaataacggcaaaaccatcattgggctgggcgacatccaggcaactgtcgatccggtcttgggccagggcgcgaacatggcgtcctatgcggcatggattctgggcgaggaaatccttgcgcactctgtctacgacctgcgcttcagcgaacacctggagcgtcgccgccaggatcgcgtgctgtgtgccacgcgatggaccaacttcactctgagcgctctctcggcacttccgccggagttcctcgccttccttcagatcctgagccagagccgtgaaatggctgatgagttcacggacaacttcaactacccggaacgtcagtgggatcgcttctccagcccggaacgtatcggacagtggtgcagtcagttcgcacccactatcgcggcctga

SEQ ID NO: 34 - protein, 415 aa, Solanum tuberosum
MKKRIGIVGAGTAGLHLGLFLRQHDVDVTVYTDRKPDEYSGLRLLNTVAHNAVTVQREVALDVNEWPSEEFGYFGHYYYVGGPQPMRFYGDLKAPSRAVDYRLYQPMLMRALEARGGKFCYDAVSAEDLEGLSEQYDLLVVCTGKYALGKVFEKQSENSPFEKPQRALCVGLFKGIKEAPIRAVTMSFSPGHGELIEIPTLSFNGMSTALVLENHIGSDLEVLAHTKYDDDPRAFLDLMLEKLGKHHPSVAERIDPAEFDLANSSLDILQGGVVPAFRDGHATLNNGKTIIGLGDIQATVDPVLGQGANMASYAAWILGEEILAHSVYDLRFSEHLERRRQDRVLCATRWTNFTLSALSALPPEFLAFLQILSQSREMADEFTDNFNYPERQWDRFSSPERIGQWCSQFAPTIAA

SEQ ID NO: 35 - DNA, 513 bp, Solanum tuberosum
atgacgttaaaaaaagatatggcggtggatatcgactccaccaacttccgccaggcggttgcattgttcgcgacgggaattgcggttctcagcgcggagactgaagagggcgatgtgcacggcatgaccgtgaacagtttcacctccatcagtctggatccgccgactgtgatggtttccctgaaatcgggccgtatgcatgagttgctgactcaaggcggacgcttcggagttagcctcttgggtgaaagccagaaggtgttctcggcattcttcagcaagcgcgcgatggatgacacgcctccccccgccttcaccattcaggccggccttcccactctgcagggcgccatggcctggttcgaatgcgaggtggagagcacggttcaagtacacgaccacacgctcttcattgcgcgcgttagcgcctgtggaacgcctgaggcgaataccccccagccgctgctgttctttgccagccgttatcacggcaacccgttgccactgaattga

SEQ ID NO: 36 - protein, 170 aa, Solanum tuberosum
MTLKKDMAVDIDSTNFRQAVALFATGIAVLSAETEEGDVHGMTVNSFTSISLDPPTVMVSLKSGRMHELLTQGGRFGVSLLGESQKVFSAFFSKRAMDDTPPPAFTIQAGLPTLQGAMAWFECEVESTVQVHDHTLFIARVSACGTPEANTPQPLLFFASRYHGNPLPLN

SEQ ID NO: 37 - DNA, 1146 bp, Sphingomonas sp. HXN-200
atgatgaacgtcgaacatatccgcccgttccgcgtcgaggtgccgcaggacgcgctcgacgatcttcgcgaccggctggcgcgcactcgctggcccgagaaggaaacggtcgacgactgggatcagggcatcccgctcgcctatgcccgcgaactcgccatctactggcgcgacgagtacgactggcggcggatcgaggcgcggctcaacacctggcccaactttctggccacagtcgacgggctcgatatccatttcctccatatccgctcggacaatcctgccgcgcggccgctggtgttgacgcacggctggccgggatcggtcctcgaatttctcgacgtcatcgaaccgctgtcggccgactatcacctcgtcatcccgtcgcttcccggtttcggtttctcgggcaagcccacccgccccggctgggatgtcgagcatatcgccgccgcgtgggacgcgctgatgcgcgcgctcggctatgaccgctattttgcgcagggcggcgactggggcagcgcggtaacctcggcgatcggcatgcaccacgccggccattgcgcgggcatccacgtcaacatggtcgtcggcgcgccgccgcccgagttgatgaacgacctcaccgacgaagagaagctctatctcgcgcgcttcggctggtatcaggcgaaggacaatggctattcgacgcagcaggcgacgcggccgcagacgatcggctatgcgctcaccgattccccggccggacagatggcgtggatcgcggagaaattccacggctggaccgattgcgggcaccagcccggcggccagtcggtcggcggccaccccgaacaggcggtctcgaaggatgcgatgctcgacacgatcagcctctattggctgaccgccagcgccgcttcgtcggcgcggctatactggcacagcttccgtcagttcgcggcgggcgagatcgacgtgccgacgggatgcagcctgttcccgaacgagatcatgcgcctgtcgcggcgctgggccgaacggcggtatcgcaacatcgtctattggagcgaagcggctcgcggcggccatttcgccgcctgggaacaacccgagctgtttgccgccgaggtccgcgcggcctttgcacagatggatctttga

SEQ ID NO: 38 - protein, 381 aa, Sphingomonas sp. HXN-200
MMNVEHIRPFRVEVPQDALDDLRDRLARTRWPEKETVDDWDQGIPLAYARELAIYWRDEYDWRRIEARLNTWPNFLATVDGLDIHFLHIRSDNPAARPLVLTHGWPGSVLEFLDVIEPLSADYHLVIPSLPGFGFSGKPTRPGWDVEHIAAAWDALMRALGYDRYFAQGGDWGSAVTSAIGMHHAGHCAGIHVNMVVGAPPPELMNDLTDEEKLYLARFGWYQAKDNGYSTQQATRPQTIGYALTDSPAGQMAWIAEKFHGWTDCGHQPGGQSVGGHPEQAVSKDAMLDTISLYWLTASAASSARLYWHSFRQFAAGEIDVPTGCSLFPNEIMRLSRRWAERRYRNIVYWSEAARGGHFAAWEQPELFAAEVRAAFAQMDL

SEQ ID NO: 39 - DNA, 1500 bp, Escherichia coli
atgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaa

SEQ ID NO: 40 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLEPLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCGKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 41 - DNA, 1503 bp, Aspergillus niger
atgagcgcgcaacctgcgcacctgtgcttccgcagtttcgtggaagcactgaaagttgataacgatctggtggaaattaataccccgatcgatccgaacctggaagcggcggcaattacccgtcgcgtgtgcgaaacgaatgataaagccccgctgtttaacaatctgattggcatgaaaaacggtctgttccgcatcctgggtgcaccgggcagtctgcgtaaaagctctgcggatcgttatggtcgtctggcacgtcatctggcactgccgccgaccgcaagcatgcgtgaaattctggataaaatgctgagtgcgagcgatatgccgccgattccgccgaccatcgtgccgacgggtccgtgtaaagaaaatagcctggatgattctgaatttgatctgaccgaactgccggttccgctgatccataaaagcgatggcggtaaatatattcagacgtacggtatgcacatcgtgcagagtccggatggcacctggacgaattggagcattgcgcgtgcgatggtgcatgataaaaaccacctgaccggtctggtgatcccgccgcagcatatttggcagatccaccagatgtggaaaaaagaaggtcgtagcgatgttccgtgggcactggcattcggcgtgccgccggcggcaattatggcgagtagcatgccgatcccggatggtgttaccgaagcgggttatgtgggcgccatgacgggctctagtctggaactggttaaatgcgataccaacgatctgtacgttccggcgacgtctgaaattgtgctggaaggcaccctgtctatcagtgaaacgggtccggaaggcccgtttggtgaaatgcatggctatattttcccgggtgatacccacctgggcgccaaatataaagtgaatcgcattacgtaccgtaacaatgcaatcatgccgatgagcagctgcggtcgcctgaccgatgaaacccatacgatgattggcagcctggcagcggccgaaatccgtaaactgtgtcagcagaacgatctgccgatcacggatgcatttgcgccgttcgaaagccaggtgacctgggttgccctgcgcgttgatacggaaaaactgcgtgcaatgaaaaccacgtctgaaggttttcgtaaacgcgtgggcgatgtggttttcaatcataaagcgggttataccattcaccgcctggtgctggttggtgatgatatcgatgtttacgaaggcaaagatgtgctgtgggccttttctacccgttgtcgcccgggtatggatgaaacgctgtttgaagatgttcgcggcttcccgctgattccgtacatgggtcatggcaacggtccggcacaccgtggcggtaaagttgttagtgatgccctgatgccgaccgaatataccacgggtcgtaattgggaagcagcggattttaaccagtcttacccggaagacctgaaacagaaagtgctggataattggaccaaaatgggcttcagtaactaa

SEQ ID NO: 42 - protein, 500 aa, Aspergillus niger
MSAQPAHLCFRSFVEALKVDNDLVEINTPIDPNLEAAAITRRVCETNDKAPLFNNLIGMKNGLFRILGAPGSLRKSSADRYGRLARHLALPPTASMREILDKMLSASDMPPIPPTIVPTGPCKENSLDDSEFDLTELPVPLIHKSDGGKYIQTYGMHIVQSPDGTWTNWSIARAMVHDKNHLTGLVIPPQHIWQIHQMWKKEGRSDVPWALAFGVPPAAIMASSMPIPDGVTEAGYVGAMTGSSLELVKCDTNDLYVPATSEIVLEGTLSISETGPEGPFGEMHGYIFPGDTHLGAKYKVNRITYRNNAIMPMSSCGRLTDETHTMIGSLAAAEIRKLCQQNDLPITDAFAPFESQVTWVALRVDTEKLRAMKTTSEGFRKRVGDVVFNHKAGYTIHRLVLVGDDIDVYEGKDVLWAFSTRCRPGMDETLFEDVRGFPLIPYMGHGNGPAHRGGKVVSDALMPTEYTTGRNWEAADFNQSYPEDLKQKVLDNWTKMGFSN

SEQ ID NO: 43 - DNA, 30 bp, Artificial Sequence
  forward primer
actgtcatgagcgcgcaacctgcgcacctg

SEQ ID NO: 44 - DNA, 35 bp, Artificial Sequence
  reverse primer
actggaattcttagttactgaagcccattttggtc

SEQ ID NO: 45 - DNA, 687 bp, Aspergillus niger
atgatgttcaactcacttctgtccggcactactacaccaaactccggccgtgcaagccctccggcaagcgaaatgccgattgataacgaccatgttgcagtcgcacgtccggcaccgcgtcgccgtcgcatcgtggttgcaatgaccggtgcaacgggtgcaatgctgggcattaaagtgctgatcgccctgcgtcgcctgaacgtcgaaacccacctggtgatgagtaaatgggcagaagctaccattaaatatgaaacggattaccatccgtcaaatgtgcgcgcgctggccgattatgttcacaacattaatgacatggcggccccggttagctctggcagctttcgtgcggatggtatgatcgtcgtgccgtgctctatgaaaaccctggcagctattcatagtggcttctgtgatgacctgatctcccgcacggcagatgtcatgctgaaagaacgtcgccgtctggtgctggttgctcgtgaaaccccgctgtccgaaatccacctgcgcaacatgctggaagttacgcgtgcaggtgctgttatttttccgccggtcccggcattctacatcaaagctggctcaattgaagatctgatcgaccagtcggtgggtcgcatgctggacctgtttgatctggacaccggcgacttcgaacgttggaatggttgggaaaaataa

SEQ ID NO: 46 - protein, 227 aa, Aspergillus niger
MFNSLLSGTTTPNSGRASPPASEMPIDNDHVAVARPAPRRRRIVVAMTGATGAMLGIKVLIALRRLNVETHLVMSKWAEATIKYETDYHPSNVRALADYVHNINDMAAPVSSGSFRADGMIVVPCSMKTLAAIHSGFCDDLISRTADVMLKERRRLVLVARETPLSEIHLRNMLEVTRAGAVIFPPVPAFYIKAGSIEDLIDQSVGRMLDLFDLDTGDFERWNGWEK

SEQ ID NO: 47 - DNA, 48 bp, Artificial Sequence
  forward primer
actggaattctaaggagatatatcatgttcaactcacttctgtccggc

SEQ ID NO: 48 - DNA, 34 bp, Artificial Sequence
  reverse primer
actgctgcagttatttttcccaaccattccaacg

SEQ ID NO: 49 - DNA, 2154 bp, Arabidopsis thaliana
atggatcaaatcgaagcaatgttgtgcggcggaggagagaagacaaaagtggcggttactacgaagactttggcagatccattgaattggggtttagcagcggatcaaatgaaaggaagtcatttagatgaagtgaagaagatggtcgaagagtatcgtagaccagtcgtgaatcttggcggagaaacactgacgatcggacaagttgctgccatctccaccgtaggaggcagcgttaaggttgagttagcggagacttcaagagccggtgtgaaagctagcagtgattgggttatggagagcatgaacaaaggtactgacagttacggagtcaccaccggctttggtgctacttctcaccggagaaccaaaaacggcaccgcattacaaacagaactcattagatttttgaacgccggaatattcggaaacacgaaggagacatgtcacacactgccgcaatccgccacaagagccgccatgctcgtcagagtcaacactcttctccaaggatactccgggatccgattcgagatcctcgaagcgattacaagtctcctcaaccacaacatctctccgtcactacctctccgtggaaccattaccgcctccgggcatctcgttcctctctcttacatcgccggacttctcaccggccgtcctaattccaaagccaccggtcccgacggtgaatcgctaaccgagaaagaagcttttgagaaagccggaatcagtactggattcttcgatttacaacctaaggaaggtttagctctcgttaatggcacggcggttggatctggaatggcgtcgatggttctattcgaagcgaatgtccaagcggtgttagcggaggttttatcagcgatcttcgcggaggttatgagcgggaaacctgagtttaccgatcatctgactcatcgtttaaaacatcatcccggacaaatcgaagcggcggcgataatggagcacatactcgacggaagctcatacatgaaattagctcaaaaggttcacgagatggatccattgcagaaaccaaaacaagatcgttacgctcttcgtacatctcctcaatggctaggtcctcaaattgaagtaatccgtcaagctacgaaatcgatagagcgtgaaatcaactccgttaacgataatccgttgatcgatgtttcgaggaacaaggcgattcacggtggtaacttccaaggaacaccaatcggagtttctatggataacacgagattggcgattgctgcgattgggaagctaatgtttgctcaattctctgagcttgttaatgatttctacaacaatggacttccttcgaatctaactgcttcgagtaatccaagtttggattatggattcaaaggagcagagattgctatggcttcttattgttctgagcttcaatacttggctaatccagtcacaagccatgttcaatcagctgagcaacataatcaagatgtgaactctcttggtttgatctcgtctcgtaaaacatctgaagctgtggatattcttaagctaatgtcaacaacgttccttgtggggatatgtcaagctgttgatttgagacatttggaggagaatctgagacaaactgtgaagaacacagtttctcaagttgctaagaaagtgttaaccactggaatcaacggtgagttacatccgtcaaggttttgcgagaaggacttgcttaaggttgttgatcgtgagcaagtgttcacgtatgtggatgatccttgtagcgctacgtacccgttgatgcagagactaagacaagttattgttgatcacgctttgtccaacggtgagactgagaagaatgcagtgacttcgatctttcaaaagattggagcttttgaagaggagcttaaggctgtgcttccaaaggaagttgaagcggctagagcggcttatgggaatggaactgcgccgattcctaaccggattaaggaatgtaggtcgtatccgttgtataggttcgtgagggaagagcttggaacgaagttgttgactggagaaaaggttgtgtctccgggagaggagtttgataaggtcttcactgctatgtgtgaaggtaaacttattgatccgttgatggattgtctcaaggaatggaacggagctccgattccgatttgctaa

SEQ ID NO: 50 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGHLVPLSYIAGLLTGRPNSKATGPDGESLTEKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 51 - DNA, 33 bp, Artificial Sequence
  forward primer
actgcatatggatcaaatcgaagcaatgttgtg

SEQ ID NO: 52 - DNA, 34 bp, Artificial Sequence
  reverse primer
actgctcgagttatttttcccaaccattccaacg

SEQ ID NO: 53 - DNA, 32 bp, Artificial Sequence
  styA forward primer
actgtcatgaaaaagcgtatcggtattgttgg

SEQ ID NO: 54 - DNA, 37 bp, Artificial Sequence
  StyA reverse primer
actggaattctcatgctgcgatagttggtgcgaactg

SEQ ID NO: 55 - DNA, 43 bp, Artificial Sequence
  styB forward primer
gaattctaaggagatttcaaatgacgctgaaaaaagatatggc

SEQ ID NO: 56 - DNA, 33 bp, Artificial Sequence
  styB reverse primer
actgggtacctcaattcagtggcaacgggttgc

SEQ ID NO: 57 - DNA, 31 bp, Artificial Sequence
  StEH reverse primer
actgctcgagttagaatttttgaataaaatc

SEQ ID NO: 58 - DNA, 49 bp, Artificial Sequence
  Aldh forward primer
cgagatcttaaggagatatataatgacagagccgcatgtagcagtatta

SEQ ID NO: 59 - DNA, 36 bp, Artificial Sequence
  Aldh reverse primer
actgctcgagttaataccgtacacacaccgacttag

(19) United States
(12) Patent Application Publication
(10) Pub. No.: US 2017/0067084 A1
(43) Pub. Date: Mar. 9, 2017

(54) PRODUCTION OF CHIRAL 1,2-AMINO ALCOHOLS AND ALPHA-AMINO ACIDS FROM ALKENES BY CASCADE BIOCATALYSIS

(71) Applicant: National University of Singapore, Singapore (SG)

(72) Inventors: Zhi Li, Singapore (SG); Shuke Wu, Singapore (SG)

(21) Appl. No.: 15/257,223

(22) Filed: Sep. 6, 2016

Related U.S. Application Data
(60) Provisional application No. 62/283,508, filed on Sep. 3, 2015.

(51) Int. Cl. C12P 13/04; C12N 9/02; C12P 13/00; C12N 9/10; C12N 15/52; C12N 15/70; C12N 9/04

(52) U.S. Cl. CPC C12P 13/04; C12N 15/70; C12N 9/0069; C12N 9/0006; C12N 9/1096; C12Y 103/00; C12Y 101/03013; C12Y 206/00; C12Y 101/99006; C12N 15/52; C12P 13/001

(57) ABSTRACT

Disclosed herein are methods of forming chiral 1,2-aminoalcohols and α-aminoacids from alkene starting materials by way of an enzymatic cascade reaction sequence that may be accomplished in a single reaction vessel without the need to isolate any intermediates. Also disclosed herein are recombinant nucleic acids, vectors and host cells for use in the methods of the invention.

### CROSS-REFERENCE TO RELATED APPLICATIONS

The present application claims priority of Provisional Application No. 62/283,508, filed on Sep. 3, 2015. The content of this prior application is hereby incorporated by reference in its entirety.

### FIELD OF INVENTION

The current invention relates to the use of a cascade reaction to transform starting materials such as alkenes, amino acids and α-carboxyalkenes into new and useful chiral compounds that may have useful industrial applications, such as starting materials for the formation of pharmaceuticals and agrichemicals.

### BACKGROUND

Sustainable manufacturing of chemicals from renewable feedstock is attracting increasing attention due to the oil depletion and global climate change. Significant progress has been made in chemical or enzymatic conversion of biomass to bulk chemicals. The advances of metabolic engineering and synthetic biology have enabled the fermentation of (hemi)cellulose-derived sugars to produce a variety of bio-based bulk chemicals. However, the fermentative production of non-natural high-value fine chemicals still faces many challenges including the lack of efficient pathways towards the non-natural chemicals. For example, chiral 1,2-amino alcohols and α-amino acids are two important classes of fine chemicals with widespread applications in many industries. However, methods to produce these chemicals (especially non-natural variations) still generally require the use of expensive and, potentially environmentally-unfriendly, chemical reagents.

Many enantiopure amino alcohols, such as phenylglycinol and phenylethanolamine, are widely used as chiral auxiliaries in asymmetric synthesis, and as intermediates for some chiral bioactive drug candidates. For example, (S)-phenylethanolamine was used to synthesize novel β₃-adrenoceptor agonists, CB2 selective agonists, and PAK Inhibitors. (R)-Phenylethanolamine is the building block of Robotnikinin, a Hedgehog signalling protein inhibitor, with significant biological and pharmaceutical potential. (S)-Phenylglycinol is a key precursor for the synthesis of chiral ligands for asymmetric annulation or cyclization, potent histone deacetylase inhibitors, and novel anti-cancer asymmetric triplex metallohelices. (R)-Phenylglycinol is also very useful for novel antibiotic pyrazolopyrimidines, EGFR inhibitor thienopyrimidines, and chiral ligands in copper-based multicomponent catalytic processes. Enantiopure 2-amino-1-phenylpropan-1-ol (norephedrine, norpseudoephedrine) is a key precursor for ephedrine, which is a sympathomimetic agent and has widespread use as an adrenergic stimulant. Another very useful chiral 1,2-amino alcohol is (1S, 2R)-1-amino-2-indanol, which is the chiral building block for anti-HIV drug Indinavir (Crixivan™).

In addition to amino alcohols, enantiopure α-amino acids are also a class of chiral chemicals with many applications. One outstanding example is (R)-phenylglycine, which is produced in more than 5000 tons per year for the manufacture of antibiotics, such as Ampicillin and Cefalexin. The unit price of (R)-phenylglycine was estimated to be US$10-20/kg, and thus the estimated yearly production of (R)-phenylglycine alone is worth about US$50-100 million. The other enantiomer, (S)-phenylglycine, plays a crucial role in the synthesis of the side chain for the anticancer drug Taxol. Besides D- and L-phenylglycine, other substituted phenylglycines are also very useful fine chemicals. For instance, (R)-p-hydroxy-phenylglycine is the key building block for Amoxicillin and Cefadroxil and is produced on a scale of several thousand tons per year. (S)-o-Chloro-phenylglycine can be used for the blockbuster drug Clopidogrel (Plavix™) synthesis. (S)-p-Fluoro-phenylglycine is a synthon for the antiemetic drug Aprepitant (Emend™). Besides phenylglycine type of amino acids, (R)-2-amino-4-phenylbutyric acid is the key chiral precursor to manufacture a group of angiotensin-converting enzyme (ACE) inhibitors (such as Enalapril, Lisinopril, and Ramipril) and anti-cancer drug Carfilzomib (Kyprolis™). Another important α-amino acid is optically pure tert-leucine for the synthesis of many protease inhibitor drugs, such as Telaprevir (Incivek™) and Atazanavir (Reyataz™).

Enantiopure, or enantioenriched, 1,2-amino alcohols can be produced by several chemical approaches. One of the most useful reactions is asymmetric aminohydroxylation (oxyamination) of olefins which utilizes expensive and toxic metal catalysts, such as osmium, rhodium, and palladium, and also requires the use of expensive complex chiral ligands. For example, the Sharpless asymmetric aminohydroxylation of alkenes employs the use of OsO₄ as a catalyst and dihydroquinidine-derived molecules as chiral ligands. Although there are several aminohydroxylation reactions that do not use toxic and expensive metals, they usually give poor enantioselectivity of the resulting 1,2-amino alcohols. In another example, a chemo-enzymatic method was used to provide access to chiral 1,2-amino alcohols. The method first involved the enzymatic reduction of a β-keto ester/amide substrate to a chiral β-hydroxy ester/amide, followed by rearrangement to produce chiral vicinal aminoalcohols. The disadvantages associated with this hybrid method are the multiple reaction steps, difficult purification and isolation of the intermediates and the less readily-available β-keto ester/amide starting materials. Recently, a novel cascade biocatalysis method to produce chiral 1,2-amino alcohol in a one-pot manner from aldehydes and α-keto acids was reported. However, the method only demonstrated the synthesis of nor(pseudo)ephedrine, and it is not considered particularly suitable for adaption to produce other 1,2-amino alcohols.

Chiral non-natural α-amino acids can also be produced by several methods. The asymmetric hydrogenation, developed by Knowles, is one of the most widely-used methods and it involves the asymmetric reduction of α,β-dehydro-α-amino acid derivatives to chiral α-amino acid derivatives using a rhodium catalyst. While this method has many industrial applications, its disadvantage lies in the use of highly expensive and toxic precious metals. Another method for the production of α-amino acid is via the Strecker synthesis from an aldehyde/ketone, a cyanide, and ammonia. Though efficient and highly selective asymmetric Strecker synthesis can be achieved by the use of chiral organo-catalysts, the reaction utilizes a very toxic cyanide salt as one of the key reactants, which is not desirable. While enzymes can provide alternate methods for the synthesis of chiral α-amino acids, many natural proteinogenic α-amino acids are typically produced from the fermentation of microbes. It may be possible to provide non-natural α-amino acids from α-keto acids with the use of transaminases, but a drawback in putting this into effect is that α-keto acids are not easily available at low cost, making it difficult to access these valuable materials cost-effectively. While the hydantoinase process, an efficient cascade biocatalysis system, is used to make enantiopure phenylglycine on an industrial scale, it involves the use of highly toxic cyanide to synthesize the substrate hydantoin.

Thus, while methods exist to manufacture the above 1,2-aminoalcohols and α-amino acids in bulk, they require the use of expensive and environmentally unfriendly catalysts and chemicals. Therefore, there remains a need for new methods to generate the compounds more cheaply and in a more environmentally-friendly manner.

### SUMMARY OF INVENTION

It has been surprisingly found that the use of more than two enzymes can be used in a cascade reaction to provide 1,2-aminoalcohols and α-amino acids in bulk. Thus, in a first aspect of the invention, there is provided a method for producing an enantiomerically pure or enantiomerically enriched 1,2-amino alcohol or α-amino acid, which method comprises subjecting an alkene starting material to multiple enzyme-catalyzed chemical transformations in a one-pot reaction system, wherein the method comprises generating a vicinal diol from the alkene and an α-hydroxyaldehyde from the vicinal diol.

In an embodiment of the first aspect of the invention, the method produces an enantiomerically pure or enantiomerically enriched 1,2-amino alcohol, which is produced using a method comprising the steps of:
(a) generating a vicinal diol from an alkene by a dihydroxylation reaction catalyzed by a dioxygenase, or by conducting an epoxidation reaction catalyzed by an epoxidase to form an epoxide and conducting a hydrolysis reaction catalyzed by an epoxide hydrolase on the epoxide;
(b) generating an α-hydroxyaldehyde or an α-hydroxyketone from the vicinal diol by an oxidation reaction catalyzed by an alcohol oxidase or alcohol dehydrogenase; and
(c) generating a 1,2-aminoalcohol from the α-hydroxyaldehyde or α-hydroxyketone by a transamination reaction catalyzed by a transaminase or a reductive amination reaction catalyzed by an amine dehydrogenase.

In further embodiments of the method that generates an enantiomerically pure or enantiomerically enriched 1,2-amino alcohol:
(a) the method may further comprise providing the alkene by generating a vinyl carboxylic acid from an α-amino acid by a deamination reaction catalyzed by an ammonia lyase and generating the alkene from the vinyl carboxylic acid in a decarboxylation reaction catalyzed by a decarboxylase;
(b) the alcohol oxidase is alditol oxidase or its mutants, or the alcohol dehydrogenase is selected from the group consisting of AlkJ from Pseudomonas putida, AlkJ homologue from Sphingomonas sp. HXN-200, dihydrodiol dehydrogenase, and mutants thereof;
(c) the amine dehydrogenase is a phenylalanine dehydrogenase, a leucine dehydrogenase or their mutants
(d) the method may comprise the use of one or more reactive components selected from the group consisting of cells, immobilized cells, cell extract, isolated enzymes and immobilized enzymes.

In further embodiments of the method that generates an enantiomerically pure or enantiomerically enriched 1,2-amino alcohol, the transaminase is a ω-transaminase. For example, the ω-transaminase is from Vibrio fluvialis, Chromobacterium violaceum or their mutants.

In yet further embodiments of the method that generates an enantiomerically pure or enantiomerically enriched 1,2-amino alcohol, the alkene may have the formula (I):

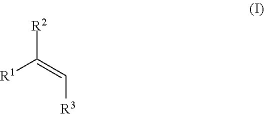

where:

R¹ to R³ independently represent H, an alkyl group, an alkenyl group, an alkynyl group, a cycloalkyl group, a cycloalkenyl group, an aryl group, an aryl alkyl group, a heterocyclic group, and a heterocyclic alkyl group, which groups are substituted or unsubstituted by one or more substituents, provided that at least one of R¹ to R³ is not H.

In an embodiment of the first aspect of the invention, the method produces an enantiomerically pure or enantiomerically enriched α-amino acid, which is produced using a method comprising the steps of:
(a) generating a vicinal diol from an alkene by a dihydroxylation reaction catalyzed by a dioxygenase, or by conducting an epoxidation reaction catalyzed by an epoxidase to form an epoxide and conducting a hydrolysis reaction catalyzed by an epoxide hydrolase on the epoxide;
(b) generating an α-hydroxyaldehyde from the vicinal diol by an oxidation reaction catalyzed by an alcohol oxidase or alcohol dehydrogenase;
(c) generating an α-hydroxy acid from the α-hydroxyaldehyde in an oxidation reaction catalyzed by an aldehyde dehydrogenase or an aldehyde oxidase;
(d) generating an α-ketoacid from the α-hydroxy acid in an oxidation reaction catalyzed by a hydroxy acid dehydrogenase or a hydroxy acid oxidase; and
(e) generating an α-amino acid from the α-ketoacid by a transamination reaction catalyzed by a transaminase or a reductive amination reaction catalyzed by an amino acid dehydrogenase.

In further embodiments of the method that generates an enantiomerically pure or enantiomerically enriched α-amino acid:
(a) the method may further comprise providing the alkene by generating a vinyl carboxylic acid from an α-amino acid by a deamination reaction catalyzed by an ammonia lyase and generating the alkene from the vinyl carboxylic acid in a decarboxylation reaction catalyzed by a decarboxylase;
(b) the alcohol oxidase is alditol oxidase or its mutants, or the alcohol dehydrogenase is selected from the group consisting of AlkJ from Pseudomonas putida, AlkJ homologue from Sphingomonas sp. HXN-200, dihydrodiol dehydrogenase, and mutants thereof;
(c) the aldehyde dehydrogenase is AlkH from Pseudomonas putida or its mutants or phenyl aldehyde dehydrogenase from Escherichia coli or its mutants, or combinations thereof;
(d) the hydroxy acid dehydrogenase mandelate dehydrogenase or its mutants, and the hydroxy acid oxidase is mandelate oxidase or its mutants, or hydroxymandelate oxidase from S. coelicolor and its mutants, or combinations thereof;
(e) the method may comprise the use of one or more reactive components selected from the group consisting of cells, immobilized cells, cell extract, isolated enzymes and immobilized enzymes.

In further embodiments of the method that generates an enantiomerically pure or enantiomerically enriched α-amino acid, the transaminase is an α-transaminase. For example, the α-transaminase is selected from one or more of the group consisting of α-transaminase (IlvE) from Escherichia coli or its mutants, α-transaminase (Tyr8) from Saccharomyces cerevisiae or its mutants, and α-transaminase (D-phenylglycine aminotransferase) from Pseudomonas stutzeri or its mutants.

In still further embodiments of the method that generates an enantiomerically pure or enantiomerically enriched α-amino acid, the alkene may have the formula (II):

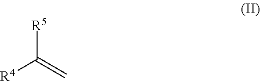

where:

R⁴ and R⁵ independently represent H, a straight chain or branched alkyl group, a straight chain or branched alkenyl group, a straight chain or branched alkynyl group, a cycloalkyl group, a cycloalkenyl group, an aryl group, an aryl alkyl group, and a heterocyclic group, which groups are substituted or unsubstituted by one or more substituents, provided that at least one of R⁴ and R⁵ is not H.

It will be appreciated that the method outlined above works by the combination of particular enzymes into a single reaction system. As such, in a second aspect of the invention, there is provided an isolated nucleic acid molecule encoding at least one catalytic enzyme selected from the group comprising:
(a) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(b) an alcohol oxidase or alcohol dehydrogenase for generating an α-hydroxyaldehyde from the vicinal diol by an oxidation reaction; and
(c) a transaminase for generating a 1,2-aminoalcohol from the α-hydroxyaldehyde by a transamination reaction; or
(d) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(e) an alcohol oxidase or alcohol dehydrogenase for generating an α-hydroxyaldehyde from the vicinal diol by an oxidation reaction;
(f) an aldehyde dehydrogenase for generating an α-hydroxy acid from the α-hydroxyaldehyde in an oxidation reaction;
(g) a hydroxy acid dehydrogenase or a hydroxy acid oxidase for generating an α-ketoacid from the α-hydroxy acid in an oxidation reaction; and
(h) a transaminase for generating an α-amino acid from the α-ketoacid by a transamination reaction.

According to an aspect of the present invention, there is provided an isolated nucleic acid molecule encoding at least one catalytic enzyme, or a variant, mutant, or fragment thereof according to any aspect of the present invention. More particularly, the present invention provides an isolated nucleic acid molecule encoding at least one heterologous catalytic enzyme selected from the group comprising:
(a) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(b) an alcohol oxidase or alcohol dehydrogenase for generating an α-hydroxyaldehyde or an α-hydroxyketone from the vicinal diol by an oxidation reaction; and
(c) a transaminase or an amine dehydrogenase for generating a 1,2-aminoalcohol from the α-hydroxyaldehyde or the α-hydroxyketone by a transamination reaction or a reductive amination; or
(d) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(e) an alcohol oxidase or alcohol dehydrogenase for generating an α-hydroxyaldehyde from the vicinal diol by an oxidation reaction;
(f) an aldehyde dehydrogenase or an aldehyde oxidase for generating an α-hydroxy acid from the α-hydroxyaldehyde in an oxidation reaction;
(g) a hydroxy acid dehydrogenase or a hydroxy acid oxidase for generating an α-ketoacid from the α-hydroxy acid in an oxidation reaction; and
(h) a transaminase or an amino acid dehydrogenase for generating an α-amino acid from the α-ketoacid by a transamination reaction or reductive amination reaction.

It will be appreciated that the isolated nucleic acid of the second aspect of the invention may encode for a plurality of catalytic enzymes of which at least one is heterologous. For example, the plurality of catalytic enzymes is arranged as at least one module selected from the group comprising:
i) a module comprising heterologous nucleic acid sequences that, when expressed, enzymatically transforms a terminal alkene to a 1,2-diol;
ii) a module comprising heterologous nucleic acid sequences that, when expressed, enzymatically transforms a 1,2-diol to a 1,2-amino alcohol;
iii) a module comprising heterologous nucleic acid sequences that, when expressed, enzymatically transforms a 1,2-diol to an α-hydroxy acid;
iv) a module comprising heterologous nucleic acid sequences that, when expressed, enzymatically transforms an α-hydroxy acid to an α-amino acid; and
v) a module comprising heterologous nucleic acid sequences that, when expressed, enzymatically transforms an L-amino acid to a terminal alkene.

In another preferred embodiment, said isolated nucleic acid molecule comprises one or more modules selected from the group comprising:
i) a module comprising heterologous nucleic acid sequences that, when expressed, enzymatically transforms a terminal alkene to a 1,2-diol and a module comprising heterologous nucleic acid sequences that, when expressed, enzymatically transforms a 1,2-diol to a 1,2-amino alcohol;
ii) a module comprising heterologous nucleic acid sequences that, when expressed, enzymatically transforms a terminal alkene to a 1,2-diol and a module comprising heterologous nucleic acid sequences that, when expressed, enzymatically transforms a 1,2-diol to an α-hydroxy acid; and
iii) a module comprising heterologous nucleic acid sequences that, when expressed, enzymatically transforms a terminal alkene to a 1,2-diol; a module comprising heterologous nucleic acid sequences that, when expressed, enzymatically transforms a 1,2-diol to a 1,2-amino alcohol and a module comprising heterologous nucleic acid sequences that, when expressed, enzymatically transforms an α-hydroxy acid to an α-amino acid; and optionally, a module comprising heterologous nucleic acid sequences that, when expressed, enzymatically transforms an L-amino acid to a terminal alkene.

In particular, the isolated nucleic acid molecule may encode:
i) at least one of SEQ ID NOs: 34, 36 and 38, variants, mutants, or fragments thereof to transform a terminal alkene to a 1,2-diol; and/or
ii) at least one of SEQ ID NOs: 2 and 40, variants, mutants, or fragments thereof to transform a 1,2-diol to a 1,2-amino alcohol; and/or
iii) at least one of SEQ ID NOs: 2, 6 and 10, variants, mutants, or fragments thereof to transform a 1,2-diol to an α-hydroxy acid; and/or
iv) at least one of SEQ ID NOs: 16, 20, 24 and 28, variants, mutants, or fragments thereof to transform an α-hydroxy acid to an α-amino acid and/or
v) at least one of SEQ ID NOs: 42, 46 and 50, variants, mutants, or fragments thereof to transform an L-amino acid to a terminal alkene.

More in particular, the isolated nucleic acid molecule may comprise:
i) at least one of SEQ ID NOs: 33, 35 and 37, variants, mutants, or fragments thereof to transform a terminal alkene to a 1,2-diol; and/or
ii) at least one of SEQ ID NOs: 1 and 39, variants, mutants, or fragments thereof to transform a 1,2-diol to a 1,2-amino alcohol; and/or
iii) at least one of SEQ ID NOs: 1, 5 and 9, variants, mutants, or fragments thereof to transform a 1,2-diol to an α-hydroxy acid; and/or
iv) at least one of SEQ ID NOs: 15, 19, 23 and 27, variants, mutants, or fragments thereof to transform an α-hydroxy acid to an α-amino acid; and/or
v) at least one of SEQ ID NOs: 41, 45 and 49, variants, mutants, or fragments thereof to transform an L-amino acid to a terminal alkene.

In a preferred embodiment, the isolated nucleic acid molecule may encode:
i) SEQ ID NOs: 34, 36, 38, 2 and 40, variants, mutants, or fragments thereof to transform a terminal alkene to a 1,2-amino alcohol; and/or
ii) SEQ ID NOs: 34, 36, 38, 2, 6 and 10, variants, mutants, or fragments thereof to transform a terminal alkene to an α-hydroxy acid; and/or
iii) SEQ ID NOs: 34, 36, 38, 2, 40, 16, 20, 24 and 28 variants, mutants, or fragments thereof to transform a terminal alkene to an α-amino acid, and/or
iv) SEQ ID NOs: 42, 46 and 50, variants, mutants, or fragments thereof to transform an L-amino acid to a terminal alkene.

Another aspect the invention provides an expression construct comprising at least one nucleic acid molecule according to any aspect of the invention and a heterologous nucleic acid sequence.

In one aspect of the invention there is provided one or more recombinant prokaryotic or eukaryotic cells selected from the group comprising bacterial cells, yeast cells, mammalian cells and insect cells, wherein said cells comprise at least one expression construct and/or heterologous nucleic acid molecule that encodes at least one catalytic enzyme required in the pathway from alkene to enantiomerically pure or enantiomerically enriched 1,2-amino alcohol or from an α-amino acid to enantiomerically pure or enantiomerically enriched 1,2-amino alcohol. Preferably said cells are recombinant bacterial cells.

In another preferred embodiment, said catalytic enzymes for use in the invention have at least 60% 65%, 70%, 75%, 80%, 85%, 90%, 95%, 96%, 97%, 98%, 99%, or more, homology or amino acid identity with at least one enzyme selected from the group comprising an alcohol dehydrogenase with amino acid sequence represented by SEQ ID NO: 2, an ω-transaminase with amino acid sequence represented by SEQ ID NO: 5, an alanine dehydrogenase with amino acid sequence represented by SEQ ID NO: 9, a styrene monooxygenase with amino acid sequence represented by SEQ ID NOs: 34 & 36, an epoxide hydrolase with amino acid sequence represented by SEQ ID NO: 38, an aldehyde dehydrogenase with amino acid sequence represented by SEQ ID NO: 40; a phenylacrylic acid decarboxylase with amino acid sequence represented by SEQ ID NOs: 42 & 45 and a phenylalanine ammonia lyase with amino acid sequence represented by SEQ ID NO: 50.

In another preferred embodiment, the one or more recombinant cells express catalytic enzymes selected from the groups comprising;
i) styrene monooxygenase, epoxide hydrolase, alcohol dehydrogenase, ω-transaminase, and alanine dehydrogenase, variants or bioactive fragments thereof for producing a 1, 2 amino-alcohol from a terminal alkene; or
ii) styrene monooxygenase, epoxide hydrolase, alcohol dehydrogenase, aldehyde dehydrogenase, hydroxy acid oxidase, α-transaminase, glutamate dehydrogenase, and catalase, variants or bioactive fragments thereof for producing an alpha amino acid from a terminal alkene and, optionally;
iii) lyase and decarboxylase, variants or bioactive fragments thereof for producing a terminal alkene from an L-amino acid.

According to another aspect of the invention there is provided a kit comprising at least one recombinant cell, expression construct or isolated nucleic acid according to any aspect of the invention.

### BRIEF DESCRIPTION OF THE DRAWINGS

FIG. 1. Shows an overall novel cascade biocatalysis route to produce chiral 1,2-amino alcohols in high e.e. from alkenes. 110: Dihydroxylation (epoxidation-hydrolysis); 120: oxidation; 130: amination.

FIG. 2 Shows an overall novel cascade biocatalysis route to produce chiral α-amino acids in high e.e. from terminal alkenes. 140: dihyroxylation (epoxidation-hydrolysis); 150: double oxidation; 160: oxidation-amination.

FIG. 3 Shows the design of four independent modular transformations. Module 1: epoxidase (EP) and epoxide hydrolase (EH) for epoxidation-hydrolysis of terminal alkene to 1,2-diol; Module 2: alcohol dehydrogenase (ADH) and aldehyde dehydrogenase (ALDH) for terminal double oxidation of 1,2-diol to α-hydroxy acid; Module 3: ADH, co-transaminase (co-TA) and alanine dehydrogenase (AlaDH) for oxidation-transamination of 1,2-diol to 1,2-amino alcohol; Module 4: hydroxy acid oxidase (HO), α-transaminase (α-TA), catalase (CAT) and glutamate dehydrogenase (GluDH) for oxidation-transamination of α-hydroxy acid to α-amino acid.

FIG. 4 Shows a targeted cascade transformation of styrenes to corresponding (S)-phenylethanol amines and (S)-phenylglycines, with recombinant E. coli strains co-expressing multiple enzymes on multiple modules.

FIG. 5 Shows a cascade biocatalysis for one-pot transformation of biobased L-phenylalanine (S)-1 into styrene (S)-3, (S)-styrene oxide (S)-4, (S)- and (R)-1-phenylethane-1,2-diol (S)-5 and (R)-5, respectively, (S)-mandelic acid (S)-7, and (S)-phenylglycine (S)-9.

FIG. 6 Shows recombinant plasmids used in this study. The recombinant plasmids were constructed on pACYCDuet-1, pCDFDuet-1, pETDuet-1, and pRSFDuet-1 (Novagen) with arrows representing promoters.

FIG. 7 Shows a cascade transformation and genetic construction of Module 1.

FIG. 8 Shows SDS-PAGE analysis of whole-cell protein of four E. coli strains containing Module 1-4, respectively. Arrows indicate the expressed enzymes. C: control; M: Marker; M1: Module 1 (StyA, SpEH); M2: (AlkJ, EcALDH); M3: (AlkJ, CvωTA, AlaDH); M4: (CAT, GluDH, HMO, EcaTA).

FIG. 9 Shows a cascade transformation and genetic construction of Module 2.

FIG. 10 Shows a cascade transformation and genetic construction of Module 3.

FIGS. 11a and 11 b Show a cascade transformation over 24 h of 40 mM (S)-phenylethane diol (PED) to (S)-phenylethanol amine (PEA) with (FIG. 11a) E. coli expressing AlkJ and CvωTA and 200 mM L-alanine; (FIG. 11b) E. coli expressing AlkJ, CvωTA, and AlaDH (Module 3) and 200 mM NH₃.

FIG. 12 Shows a cascade transformation and genetic construction of Module 4.

FIGS. 13a and 13b Oxidation of 50 mM (S)-mandelic acid (MA) to phenylglyoxylic acid (PGA) over 24 h with (FIG. 13a) E. coli expressing HMO; (FIG. 13b) E. coli expressing MDH.

FIGS. 14a and 14b Show amination of 50 mM phenylglyoxylic acid (PGA) over 24 h with (FIG. 14a) E. coli expressing α-TA and 200 mM glutamate; (FIG. 14b) E. coli expressing α-TA and/or GluDH and 200 mM NH₃.

FIG. 15 Shows a cascade transformation of 45 mM (S)-mandelic acid (MA) to (S)-phenylglycine (PG) over 32 h with E. coli co-expressing HMO, EclIvE, GluDH, KatE (Module 4).

FIG. 16 Shows a cascade transformation of styrene to chiral (S)-phenylethanol amine with recombinant E. coli co-expressing multiple enzymes on Module 1 and Module 3.

FIGS. 17a and 17b Show a 3-dimensional representation of a cascade transformation of 50 mM styrene to (S)-phenylethanol amine (PEA) with various amount of glucose and ammonia using (FIG. 17a) E. coli (AR); (FIG. 17b) E. coli (CE).

FIG. 18 Shows a cascade transformation of 50 mM styrene to (S)-phenylethanol amine (PEA) with twelve different E. coli strains each containing Module 1 and 3, co-expressing SMO, SpEH, AlkJ, CvωTA, and AlaDH. Values are average of three independent results.

FIG. 19 Shows SDS-PAGE analysis of whole-cell protein of twelve E. coli strains each containing Module 1 and 3, co-expressing SMO, SpEH, AlkJ, CvωTA, and AlaDH.

FIG. 20 Shows a time course of biotransformation over 24 h of 60 mM styrene (Sty) to (S)-phenylethanol amine (PEA) with E. coli (AE) cells (15 g cdw/L) in a two-liquid-phase system.

FIG. 21 Shows a cascade transformation of styrene to chiral (S)-phenylglycine with recombinant E. coli co-expressing multiple enzymes on Module 1, Module 2, and Module 4.

FIG. 22 Shows a cascade transformation of 50 mM styrene to (S)-phenylglycine (PG) with eight different E. coli strains each containing Module 1, 2, and 4, co-expressing SMO, SpEH, AlkJ, EcALDH, HMO, EclIvE, GluDH, and CAT. Values are average of three independent results.

FIG. 23 Shows a SDS-PAGE analysis of whole-cell protein of eight E. coli strains each containing Module 1, 2, and 4, co-expressing SMO, SpEH, AlkJ, EcALDH, HMO, EclIvE, GluDH, and CAT.

FIG. 24 Shows a 24 h time course of biotransformation of 60 mM styrene (Sty) to (S)-phenylglycine (PG) with E. coli (ARC) cells (15 g cdw/L) in a two-liquid-phase system.

FIG. 25 Shows time curves of cascade biotransformation of L-phenylalanine to styrenes with E. coli (pRSF-PAL-PAD). Error bars represent the standard deviation of triplicated biotransformations.

FIGS. 26a and 26b Show (FIG. 26a) biotransformation of 100 mM L-phenylalanine (S)-1 to (S)-styrene oxide 4 with 12 different E. coli strains (10 g cdw L⁻¹ resting cells) coexpressing PAL, PAD, and SMO; (FIG. 26b) biotransformation of 120 mM (S)-1 to (S)-4 with the best three E. coli strains (15 g cdw L⁻¹ resting cells). The reaction was performed in a two-phase system containing KP buffer (200 mM, pH 8, 2% glucose) and n-hexadecane (1:1) and shaken at 250 rpm and 30° C.

FIGS. 27a and 27b Show (FIG. 27a) biotransformation of 100 mM L-phenylalanine (S)-1 to (R)-1-phenylethane-1,2-diol 5 with 12 different E. coli strains (10 g cdw L⁻¹ resting cells) coexpressing PAL, PAD, SMO, and StEH; (FIG. 27b) biotransformation of 120 mM (S)-1 to (R)-5 with the best three E. coli strains (15 g cdw L⁻¹ resting cells). The reaction was performed in a two-phase system containing KP buffer (200 mM, pH 8, 2% glucose) and n-hexadecane (1:1) and shaken at 250 rpm and 30° C.

FIGS. 28a and 28b Show (FIG. 28a) biotransformation of 100 mM L-phenylalanine (S)-1 to (S)-1-phenylethane-1,2-diol 5 with 12 different E. coli strains (10 g cdw L⁻¹ resting cells) coexpressing PAL, PAD, SMO, and SpEH; (FIG. 28b) biotransformation of 120 mM (S)-1 to (S)-5 with the best three E. coli strains (15 g cdw L⁻¹ resting cells). The reaction was performed in a two-phase system containing KP buffer (200 mM, pH 8, 2% glucose) and n-hexadecane (1:1) and shaken at 250 rpm and 30° C.

FIGS. 29a and 29b Show (FIG. 29a) biotransformation of 100 mM L-phenylalanine (S)-1 to (S)-mandelic acid 7 with 24 different E. coli strains (10 g cdw L⁻¹ resting cells) coexpressing PAL, PAD, SMO, SpEH, AlkJ, and EcALDH; (FIG. 29b) biotransformation of 120 mM (S)-1 to (S)-7 with the best three E. coli strains (15 g cdw L⁻¹ resting cells). The reaction was performed in a two-phase system containing KP buffer (200 mM, pH 8, 0.5% glucose) and n-hexadecane (1:1) and shaken at 250 rpm and 30° C. The biotransformation in b) was performed in triplicate, and error bars show ±s.d.

FIGS. 30a and 30b Show (FIG. 30a) biotransformation of 30 mM L-phenylalanine (S)-1 to (S)-phenylglycine 9 with 24 different E. coli strains (10 g cdw L⁻¹ resting cells) coexpressing PAL, PAD, SMO, SpEH, AlkJ, EcALDH, HMO, EcaTA, GluDH, and CAT; (FIG. 30b) biotransformation of 50 mM (S)-1 to (S)-9 with the best three E. coli strains (15 g cdw L⁻¹ resting cells). The reaction was performed in a two-phase system containing KP buffer (200 mM, pH 8, 0.5% glucose) and n-hexadecane (1:1) and shaken at 250 rpm and 30° C. 200 mM NH₃/NH₄Cl and 2% glucose was added at 10 h. The biotransformation in FIG. 30b was performed in triplicate, and error bars show ±s.d.

### DESCRIPTION

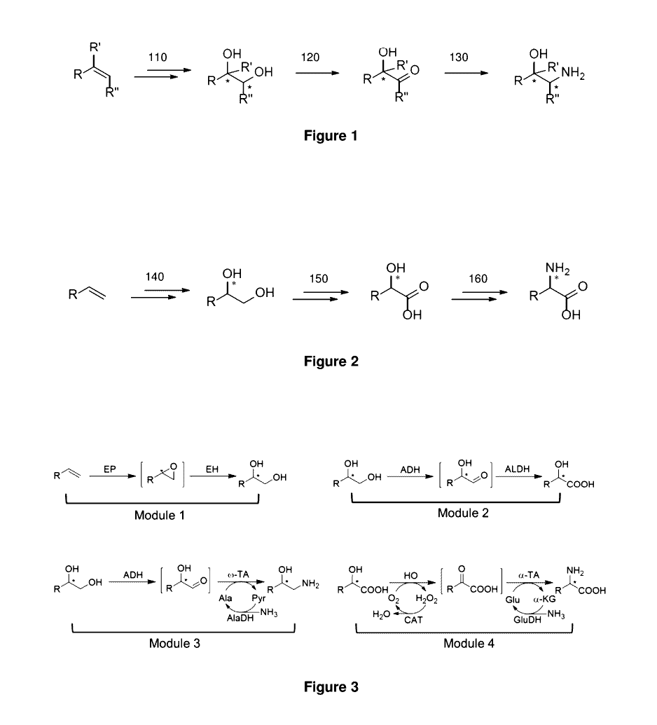

Novel biocatalytic routes to produce enantiomerically pure or enriched 1,2-amino alcohols (FIG. 1) and α-amino acids (FIG. 2) from the readily available and cheap alkenes are provided herein. For both products, the first step involves the dihydroxylation of alkenes to vicinal diols with dioxygenase in a single step or with epoxidase-hydrolase (a two-step process involving epoxidation and hydrolysis).

In order to provide the amino alcohol, an oxidation-amination cascade is used to convert one of hydroxyl groups of the vicinal diol into an amino group, which cascade involves the use of a dehydrogenase/oxidase (e.g. to form an aldehyde or ketone) and a transaminase (e.g. a ω-transaminase to form the amino group from the aldehyde or ketone group) to form the desired 1,2-amino alcohol in an enantiopure or enantioenriched form (FIG. 1).

To produce an α-amino acid, a double oxidation of a terminal vicinal diol is conducted to provide an α-hydroxy acid (e.g. using suitable oxidases). This is followed by an oxidation-amination cascade that converts the α-hydroxyl group to α-amino group by use of a dehydrogenase/oxidase and a α-transaminase (e.g. an α-transaminase) to form an α-amino acid (FIG. 2) in an enantiopure or enantioenriched form. To obtain products in high enantiomeric and diastereomeric purities, the enzymes responsible for these reactions need to demonstrate high stereo- and regio-selectivity.

These chemical transformations can be classified into four different modular transformations (FIG. 3): Module 1 is dihydroxylation of alkene (epoxidation-hydrolysis); Module 2 is double oxidation of diol to α-hydroxy acid; Module 3 is oxidation-amination of diol to 1,2-amino alcohol; Module 4 is oxidation-amination of α-hydroxy acid to α-amino acid. As such, the combination of Module 1 and Module 3 enables the conversion of alkene to 1,2-amino alcohol, and the combination of Module 1, Module 2, and Module 4 enables conversion of alkene to α-amino acid.

As disclosed herein, these multiple reactions may be performed simultaneously or sequentially in one reaction vessel, to allow for the green, efficient, and economical production of chiral 1,2-amino alcohols and α-amino acids directly from alkenes. Such, one-pot cascade reactions may avoid the expensive and energy-consuming isolation and purification of intermediates, minimize waste generation, and overcome the possible thermodynamic hurdles normally encountered in traditional multi-step synthesis. For example, multiple enzymes may be co-expressed inside one recombinant microbe strain, and the whole cells of the strain may be directly applied as catalysts for a series of cascade reactions in one pot. Alternatively, the enzymes could be separately expressed in cells of different strains, purified individually, or immobilized (the purified enzymes or cells containing all or some of said enzymes). In any event, the biocatalysts (enzymes, cells, immobilized enzymes, and immobilized cells) can be mixed together in any suitable combination to effect the one pot transformation of an alkene into a 1,2-amino alcohol or an α-amino acid.

As discussed hereinbefore, there is provided a method for producing an enantiomerically pure or enantiomerically enriched 1,2-amino alcohol or α-amino acid, which method comprises subjecting an alkene starting material to multiple enzyme-catalyzed chemical transformations in a one-pot reaction system, wherein the method comprises generating a vicinal diol from the alkene and an α-hydroxyaldehyde from the vicinal diol.

Suitable substrate alkenes can be manufactured on very large scales in the petrochemical industry (e.g. by hydrocarbon cracking), and so form easily available and cheap starting materials for organic synthesis of the type discussed herein. For example, many aromatic and aliphatic alkenes are produced in very large amounts and at very low price. As discussed hereinbelow, styrenes and substituted styrenes are very useful substrates to produce various chiral aromatic 1,2-amino alcohols (such as phenylethanol amine, phenylglycinol, nor(pseudo)ephedrine) and α-amino acids (such as phenylglycine) in high enantiomeric purity. Other readily available alkenes that may be useful include aliphatic alkenes such as 1-hexene, which could serve as the starting material to produce different chiral aliphatic 1,2-amino alcohols and α-amino acids. Other alkenes that may be useful include, but are not limited to, 3,3-dimethyl-1-butene, indene, and 4-phenyl-1-butene, as the resulting products following the enzymatic cascades disclosed herein enable the formation of tert-leucine, (1S, 2R)-1-amino-2-indanol, and (R)-2-amino-4-phenylbutyric acid in high enantiomeric purity, which are all desirable starting materials for the efficient chemical synthesis of commercially and/or pharmaceutically important compounds.

While the use of suitable substrate alkenes generated from the petrochemical industry is envisaged, the current invention also allows for the conversion of suitable amino acids and/or carboxyalkenes into suitable substrate alkenes by way of further enzyme-catalyzed transformations which will be discussed hereinbelow. This may enable access to alkenes that are otherwise difficult to obtain access to and provide a greater pool of possible alkene substrates for use in the enzyme-catalyzed transformations described herein.

It will be understood that the terms “enantiomerically pure” and “enantiomerically enriched” refer to enantiomers of a compound. “Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and L or (+) and (−) are employed to designate the sign of rotation of plane-polarized light by the compound, with (−) or l (L) meaning that the compound is levorotatory. A compound prefixed with (+) or d (D) is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

When referred to herein, the term “enantiomerically enriched” may refer to an enantiomeric excess of 50% or more. For example, the methods disclosed herein may provide a final product having an enantiomeric excess of 60%, 70%, 80%, 90%, 95%, 98%, or 99% or more. In embodiments of the invention, only one enantiomer or diastereomer of a chiral compound is provided by the process described herein (i.e. the compound is “enantiomerically pure”).

The vicinal diol intermediate referred to herein may be obtained from an alkene by a dihydroxylation reaction catalyzed by a dioxygenase. Alternatively, the vicinal diol intermediate may be obtained from an alkene by a two-step process involving as a first step an epoxidation reaction catalyzed by an epoxidase and as a second step a hydrolysis reaction on the epoxide formed in the first step, which is catalyzed by an epoxide hydrolase. Suitable enzyme catalysts for the epoxidation step include, but are not limited to monooxygenases and peroxidases. For example, suitable monooxygenases include, but are not limited to, a styrene monooxygenase or its mutants (e.g. styrene monooxygenase from Pseudomonas sp. VLB120), a P450 monooxygenase (e.g. P450pyr from Sphingomonas sp. HXN-200), and an alkene monooxygenase. Suitable epoxide hydrolases include, but are not limited to, SpEH from Sphingomonas sp. HXN-200 or its mutants, StEH from Solanum tuberosum or its mutants, AnEH from Aspergillus niger or its mutants.

In order to provide an enantiomerically pure or enantiomerically enriched 1,2-amino alcohol, the method may comprise the steps of:
(a) generating a vicinal diol from an alkene by a dihydroxylation reaction catalyzed by a dioxygenase, or by conducting an epoxidation reaction catalyzed by an epoxidase to form an epoxide and conducting a hydrolysis reaction catalyzed by an epoxide hydrolase on the epoxide;
(b) generating an α-hydroxyaldehyde or a or an α-hydroxyketone from the vicinal diol by an oxidation reaction catalyzed by an alcohol oxidase or alcohol dehydrogenase; and
(c) generating a 1,2-aminoalcohol from the α-hydroxyaldehyde or α-hydroxyketone by a transamination reaction catalyzed by a transaminase or a reductive amination reaction catalyzed by an amine dehydrogenase.

Enzymes and enzymatic combinations suitable for use in the generation of vicinal diols from alkenes are discussed hereinbefore.

A suitable alcohol oxidase that may be used herein includes, but it not limited to, alditol oxidase or its mutants. Suitable alcohol dehydrogenases that may be used herein include, but are not limited to, AlkJ from Pseudomonas putida, AlkJ homologue from Sphingomonas sp. HXN-200, dihydrodiol dehydrogenase, and mutants thereof. Suitable amine dehydrogenases include, but are not limited to, phenylalanine dehydrogenase, a leucine dehydrogenase or their mutants.

In order to provide a 1,2-amino alcohol, the alkene must necessarily be an alkene that has at least one hydrogen atom on an sp² carbon atom involved in the formation of the carbon to carbon double bond. As such, suitable alkenes can be represented by formula (I):

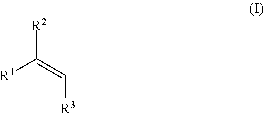

where:

R¹ to R³ independently represent H, an alkyl group, an alkenyl group, an alkynyl group, a cycloalkyl group, a cycloalkenyl group, an aryl group, an aryl alkyl group, a heterocyclic group, and a heterocyclic alkyl group, which groups are substituted or unsubstituted by one or more substituents, provided that at least one of R¹ to R³ is not H.

It will be appreciated that the alkenes of formula (I) may also be generated by forming a vinyl carboxylic acid from an α-amino acid by a deamination reaction catalyzed by an ammonia lyase and generating the alkene from the vinyl carboxylic acid in a decarboxylation reaction catalyzed by a decarboxylase. It will also be appreciated that the alkenes of formula (I) may also be generated directly from vinyl carboxylic acid in a decarboxylation reaction catalyzed by a decarboxylase.

Unless otherwise stated, the term “alkyl” refers to an unbranched or branched, saturated or unsaturated hydrocarbyl radical (so forming, for example, an alkenyl or alkynyl), which may be substituted or unsubstituted (with, for example, one or more halogen atoms). The alkyl group may be C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). The terms “alkenyl” and “alkynyl” are to be interpreted accordingly.

Unless otherwise stated, the term “cycloalkyl” refers to an unbranched or branched, saturated or unsaturated hydrocarbyl radical (so forming, for example, a cycloalkenyl group) that may be substituted or unsubstituted. The cycloalkyl group may be C₃₋₁₂ cycloalkyl and, more preferably, C₅₋₁₀ (e.g. C₅₋₇) cycloalkyl. The term “cycloalkenyl” is to be interpreted accordingly.

The term “halogen”, when used herein, includes fluorine, chlorine, bromine and iodine.

The term “aryl” when used herein includes C₆₋₁₄ (such as C₆₋₁₃ (e.g. C₆₋₁₀)) aryl groups that may be substituted or unsubstituted. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Most preferred aryl groups include phenyl.

When used herein, the term “aryl alkyl” is to be interpreted in line with the definitions provided hereinbefore for “alkyl” and “aryl”, where the point of attachment of the group to the rest of the compound of formula (I) or (II) is through the alkyl portion of the aryl alkyl group.

When used herein, the term “heterocyclic” refers to a fully saturated, partly unsaturated, wholly aromatic or partly aromatic ring system in which one or more (e.g. one to four) of the atoms in the ring system is other than carbon (i.e. a heteroatom, which heteroatom is preferably selected from N, O and S), and in which the total number of atoms in the ring system is between three and twelve (e.g. between five and ten). The heterocyclic groups may be substituted or unsubstituted. Heterocyclic groups that may be mentioned include 7-azabicyclo[2.2.1]heptanyl, 6-azabicyclo[3.1.1]heptanyl, 6-azabicyclo[3.2.1]octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thirranyl, thiolanyl, thiomorpholinyl, trithianyl (including 1,3,5-trithianyl), tropanyl, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heterocyclic groups may, where appropriate, be located on any atom in the ring system including a heteroatom. The point of attachment of heterocyclic groups may be via any atom in the ring system including (where appropriate) a heteroatom (such as a nitrogen atom), or an atom on any fused carbocyclic ring that may be present as part of the ring system. Heterocyclic groups may also be in the N- or S-oxidised form. Heterocyclic groups that may be mentioned herein include cyclic amino groups such as pyrrolidinyl, piperidyl, piperazinyl, morpholinyl or a cyclic ether such as tetrahydrofuranyl.

When used herein, the term “heterocyclic alkyl” is to be interpreted in line with the definitions provided hereinbefore for “alkyl” and “heterocyclic”, where the point of attachment of the group to the rest of the compound of formula (I) or (II) is through the alkyl portion of the heterocyclic alkyl group.

The substituents mentioned herein may be substituted or unsubstituted. When the substituents are substituted, they may be substituted with one or more of the groups selected from the group of halogen (e.g., a single halogen atom or multiple halogen atoms forming, in the latter case, groups such as CF₃ or an alkyl group bearing Cl₃), cyano, nitro, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and R_{d} are independently hydrogen, alkyl, cycloalkyl, heterocycle, aryl, or said R_{b} and R_{c} together with the N to which they are bonded optionally form a heterocycle; and Rₑ is alkyl, cycloalkyl, alkenyl, cycloalkenyl, alkynyl, heterocycle, or aryl. It will be appreciated that these substituted groups may be unsubstituted or are themselves substituted with one or more halogen atoms.

For the avoidance of doubt, in cases in which the identity of two or more substituents in a compound of formula (I) may be the same, the actual identities of the respective substituents are not in any way interdependent unless otherwise specified.

Particular alkenes of formula (I) that may be mentioned herein includes, but it not limited to, styrene, [(E)-prop-1-enyl]benzene, [(Z)-prop-1-enyl]benzene, 1H-indene, allylbenzene, but-3-enylbenzene, 3,3,-dimethylbut-1-ene and hex-1-ene. 1,2-Amino alcohols that may be mentioned herein as the products of the processes described herein include, but are not limited to enantiomerically pure or enriched 2-amino-1-phenyl-ethanol (R or S), 2-amino-1-phenyl-propan-1-ol (S,S; R,R; R,S; or S,R), 2-amino-2-phenyl-ethanol (R or S), 1-amino-1-phenyl-propan-2-ol (S,S; R,R; R,S; or S,R), and 1-aminoindan-2-ol (S,S; R,R; R,S; or S,R). It will be appreciated that the alkenes and 1,2-aminoalcohols described directly above may be unsubstituted or substituted as described hereinbefore for compounds of formula (I).

In order to provide an enantiomerically pure or enantiomerically enriched α-amino acid, the method may comprise the steps of:
(a) generating a vicinal diol from an alkene by a dihydroxylation reaction catalyzed by a dioxygenase, or by conducting an epoxidation reaction catalyzed by an epoxidase to form an epoxide and conducting a hydrolysis reaction catalyzed by an epoxide hydrolase on the epoxide;
(b) generating an α-hydroxyaldehyde from the vicinal diol by an oxidation reaction catalyzed by an alcohol oxidase or alcohol dehydrogenase;
(c) generating an α-hydroxy acid from the α-hydroxyaldehyde in an oxidation reaction catalyzed by an aldehyde dehydrogenase or an aldehyde oxidase;
(d) generating an α-ketoacid from the α-hydroxy acid in an oxidation reaction catalyzed by a hydroxy acid dehydrogenase or a hydroxy acid oxidase; and
(e) generating an α-amino acid from the α-ketoacid by a transamination reaction catalyzed by a transaminase or a reductive amination reaction catalyzed by an amino acid dehydrogenase.

Enzymes and enzymatic combinations suitable for use in the generation of vicinal diols from alkenes are discussed hereinbefore.

A suitable alcohol oxidase that may be used herein is alditol oxidase or its mutants. Suitable alcohol dehydrogenases that may be mentioned herein include, but are not limited to, AlkJ from Pseudomonas putida, AlkJ homologue from Sphingomonas sp. HXN-200, dihydrodiol dehydrogenase, and mutants thereof. Suitable aldehyde dehydrogenases include, but are not limited to AlkH from Pseudomonas putida or its mutants, phenyl aldehyde dehydrogenase from Escherichia coli or its mutants and combinations thereof. A suitable hydroxy acid dehydrogenase is, but is not limited to, mandelate dehydrogenase or its mutants. Suitable hydroxy acid oxidases include, but are not limited to, mandelate oxidase or its mutants, hydroxymandelate oxidase from S. coelicolor and its mutants and combinations thereof. Suitable amino acid dehydrogenases include, but are not limited to, D-amino acid dehydrogenase and L-amino acid dehydrogenase.

In order to provide an α-amino acid, the alkene must necessarily be an alkene that has at least two hydrogen atoms on a sp² carbon atom involved in the formation of the carbon to carbon double bond. As such, suitable alkenes can be represented by formula (II):

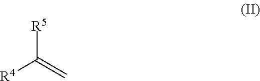

where:

R⁴ and R⁵ independently represent H, a straight chain or branched alkyl group, a straight chain or branched alkenyl group, a straight chain or branched alkynyl group, a cycloalkyl group, a cycloalkenyl group, an aryl group, an aryl alkyl group, and a heterocyclic group, which groups are substituted or unsubstituted by one or more substituents, provided that at least one of R⁴ and R⁵ is not H.

It will be appreciated that the alkenes of formula (II) may also be generated by forming a vinyl carboxylic acid from an α-amino acid by a deamination reaction catalyzed by an ammonia lyase and generating the alkene from the vinyl carboxylic acid in a decarboxylation reaction catalyzed by a decarboxylase. It will also be appreciated that the alkenes of formula (I) may also be generated directly from vinyl carboxylic acid in a decarboxylation reaction catalyzed by a decarboxylase.

When used in the context of formula (II), the terms “alkyl”, “alkenyl”, “alkynyl”, “cycloalkyl”, “halogen”, “aryl”, “aryl alkyl”, “heterocyclic” and “heterocyclic alkyl” are as defined hereinbefore for the compounds of formula (I). For the avoidance of doubt, the listing of substituted substituents presented hereinbefore applies to the compounds of formulae (I) and (II).

For the avoidance of doubt, in cases in which the identity of two or more substituents in a compound of formula (II) may be the same, the actual identities of the respective substituents are not in any way interdependent unless otherwise specified.

Particular alkenes of formula (II) that may be mentioned herein includes, but it not limited to, styrene, allylbenzene, but-3-enylbenzene, 3,3,-dimethylbut-1-ene and hex-1-ene. α-Amino acids that may be mentioned herein as the products of the processes described herein include, but are not limited to enantiomerically pure or enriched phenylglycine (R or S), phenylalanine (R or S), 2-amino-4-phenylbutanoic acid (R or S), 3-methylvaline (R or S), and norleucine (R or S). It will be appreciated that the alkenes and α-amino acids described directly above may be unsubstituted or substituted as described hereinbefore for compounds of formula (II).

The transamination reactions mentioned herein may be catalyzed by a transaminase. In order to function, the transaminase may require the presence of a co-reactant, such as a suitable nitrogen-containing reactant, which may be a suitable amino acid (e.g. alanine or glutamate). The co-reactant may be provided in at least a stoichiometric amount or it may be provided in a sub-stoichiometric amount in the form of the co-reactant or in a precursor/sideproduct that may be (re)generated by a further enzyme to provide the co-reactant, which further enzyme may require the presence of a nitrogen source (e.g. ammonia) to effect the (re)generation. For example, when the transaminase is an α-transaminase (e.g. selected from one or more of the group consisting of α-transaminase (IlvE) from Escherichia coli or its mutants, α-transaminase (Tyr8) from Saccharomyces cerevisiae or its mutants, and α-transaminase (D-phenylglycine aminotransferase) from Pseudomonas stutzeri or its mutants), the co-reactant may be alanine and the side product of the transamination reaction is pyruvate. When alanine or pyruvate are provided in sub-stoichiometric quantities, the further enzyme may be a dehydrogenase (e.g. alanine dehydrogenase) and the nitrogen source may be ammonia (in a stoichiometric amount), such that alanine is (re)generated in sufficient quantities to ensure that the transamination reaction may proceed to completion. When the transaminase is a ω-transaminase (e.g. a ω-transaminase from Chromobacterium violaceum or its mutants) the co-reactant may be glutamate and the side product of the transamination reaction is α-ketoglutarate. When alanine or α-ketoglutarate are provided in sub-stoichiometric quantities, the further enzyme may be a dehydrogenase (e.g. glutarate dehydrogenase) and the nitrogen source may be ammonia (in a stoichiometric amount), such that glutarate is (re)generated in sufficient quantities to ensure that the transamination reaction may proceed to completion.

Certain oxidation reactions used herein may generate peroxides (e.g. hydrogen peroxide) that may cause cell death or denature other enzymes if not modulated, and thus prevent the reaction cascade from completion. Given this, when a peroxide is produced, a further enzyme may be added to the reaction system in order to decompose the peroxide to oxygen and water. For example, the enzyme catalyzed oxidation of an α-hydroxyacid to an α-ketoacid may be conducted using hydroxymandelate oxidase, which may result in the generation of peroxide that is in turn converted to oxygen and water by the presence of a catalase (e.g. a catalase from E. coli).

The methods described hereinbefore make use of enzymes to catalyse a sequence of reactions. While these reactions may be performed individually or, more particularly, two or more of them in combination, it is particularly preferred that all of the reactions are combined into a cascade reaction sequence that provides the product from the initial starting material in one pot, thereby eliminating the need for isolation of the intermediates and, potentially, increasing the overall yield of the reaction sequence. These cascade reactions may involve the use of one or more reactive components selected from the group consisting of cells, immobilized cells, cell extracts, isolated enzymes and immobilized enzymes in said reaction vessel.

When microorganisms are used in the processes described herein, they may be wild type strains containing one or more of the necessary enzymes for the cascade reactions to take place. Additionally or alternatively, the microorganisms may be several recombinant E. coli strains expressing enzymes individually, several recombinant E. coli strains expressing multiple enzymes for individual reaction sequences (e.g. separate E. coli strains expressing multiple enzymes for module A, module B etc) or a single recombinant E. coli strain co-expressing multiple enzymes that are used to catalyze all of the reaction steps in the cascade reaction sequence to provide the desired product. When the microorganisms are recombinant E. coli strains, the E. coli strains may comprise one or more T7 expression plasmids and systems. While the process may involve the microorganism cells moving freely in a vehicle, they may also be immobilized onto a solid support. In additional or alternative embodiments, the method may involve the use of cell extracts of one or more microorganisms or isolated enzymes necessary for the reactions, which extracts and enzymes may also be immobilized onto a solid support or free-moving in a vehicle. It will be appreciated that any technically sensible combination of the above microorganisms, cell extracts and enzymes (both on solid support or in a vehicle) may be used. That is, the enzyme catalyzed transformations discussed herein may be conducted using a mixture of two or more of cells, immobilized cells, cell extract, isolated enzymes and immobilized enzymes. For example, a one-pot reaction system may comprise wild type cells, recombinant E. coli strains and isolated enzymes in combination, with the isolated enzymes immobilized onto a solid support. More particularly, the one-pot reaction system may comprise one or more recombinant E. coli strains.

Suitable solid supports that may be mentioned herein include, but are not limited to, inorganic carriers such as porous glass, SiO₂, alumosilica or ion-oxides; organic carriers with natural origin such as polysaccharides (Agarose), crosslinked dextrans (Sepharose) or cellolose; organic synthetic carriers such as acrylate-derivatives (co-polymers), acrylamide derivatives (co-polymers), vinylacetate derivatives (co-polymers), maleic acid anhydride derivatives, polyamides, polystyrene derivatives, polypropylenes or polymer-coated ion oxide particles.

Suitable vehicles for the reactions mentioned herein include, but are not limited to, aqueous buffer (e.g. phosphate buffer, citrate buffer, Tris buffer and HEPES buffer); a biphase system (e.g. an aqueous: ionic liquid system and an aqueous: organic system, such as an aqueous: hexadecane system).

The pH used in the methods described herein may be any suitable pH where the cells, immobilized cells, cell extract, isolated enzymes and immobilized enzymes are able to perform the necessary catalytic functions. A suitable pH may be from 3 to 12, preferably from 6 to 9. The temperature used in the methods described herein may be any suitable temperature where the cells, immobilized cells, cell extract, isolated enzymes and immobilized enzymes are able to perform the necessary catalytic functions. A suitable temperature may be from 0° C. to 90° C., preferably from 20° C. to 40° C.

For example, in an embodiment of the invention, all the enzymes responsible for the reactions are co-expressed in one recombinant E. coli strain. To construct the recombinant biocatalyst, the enzymes are cloned as several artificial operons or separately on one plasmid or several compatible plasmids. After transforming the plasmids into the E. coli strain, the multiple enzymes are co-expressed and the whole recombinant cells serve as a biocatalyst for the cascade reactions. The expression level of multiple enzymes could be adjusted and optimized for efficient cascade transformation without significant accumulation of intermediates. There are many methods to tune the expression level of multiple enzymes: using plasmids with different copy numbers, different inducer or different concentration of inducer, promoters with different strength, and different non-coding sequence (e.g. ribosome binding sites).

In embodiments of the invention, the cascade transformations may be best performed in the aqueous phase. For low concentration biotransformation, aqueous one phase system fulfils the requirement and can achieve the final product easily. However, the alkene substrates are generally hydrophobic (limited solubility in aqueous phase), toxic to the cells and may inhibit the enzyme. Thus, organic:aqueous two-phase reaction system is a better choice for high-concentration biotransformation. The alkenes and intermediate epoxides have higher solubility in the organic phase, while the diols, amino alcohols, amino acids, cells, and enzymes are mostly in the aqueous phase. By applying the two-phase reaction system, the problems of low solubility and inhibition of substrates can be overcome. In addition, the products, 1,2-amino alcohols and α-amino acids, are easily separated from the unreacted substrates and some intermediates, which will significant facilitate the downstream purification and isolation.

Other forms of biocatalyst can also be applied to synthesize the chiral compounds in high e.e., which include isolated enzyme, enzymes immobilized on nano or micro size support (such as magnetic nano particles) to increase their stability and reusability, wild type microbial cells, and recombinant cells immobilized on some carriers. By utilizing isolated enzymes, immobilized enzymes or immobilized cells, the cascade biocatalysis can be performed to produce the chiral compounds in high e.e. and good yield. A mixture of different forms of biocatalyst is also a suitable system to carry out the cascade biocatalysis.

In another aspect, the invention includes, vectors, preferably expression vectors, comprising at least one nucleic acid encoding at least one catalytic enzyme described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

The terms ‘variant’ and ‘mutant’ are used interchangeably herein. The at least one nucleic acids encoding at least one catalytic enzyme may encode a variant or mutant of the exemplified catalytic enzyme which retains activity. A “variant” of a catalytic enzyme, as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing catalytic activity may be found using computer programs well known in the art, for example, DNASTAR software. In some embodiments, variant enzymes are at least 60%, 65%, 70%, 75%, 80%, 85%, 90%, 95%, 96%, 97%, 98%, 99%, or more, homologous or identical at the amino acid level to an exemplary amino acid sequence described herein (e.g., catalase, alcohol dehydrogenase, α-transaminase) or a functional fragment thereof—e.g., over a length of about: 50, 55, 60, 65, 70, 75, 80, 85, 90, 95, or 100% of the length of the mature reference sequence. An exemplary catalase is from the katE gene (SEQ ID NO: 27) from E. coli which encodes the protein sequence (SEQ ID NO: 28), whereas an exemplary alcohol dehydrogenase is represented by SEQ ID NO: 2, and an exemplary α-transaminase is represented by SEQ ID NO: 5.

A vector can include one or more catalytic enzyme nucleic acid(s) in a form suitable for expression of the nucleic acid(s) in a host cell. Preferably the recombinant expression vector includes one or more regulatory sequences operatively linked to the nucleic acid sequence(s) to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences such as the T7 IPTG-inducible promoters disclosed in the Examples herein. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., catalytic enzyme proteins, fusion proteins, and the like).

The recombinant expression vectors of the invention can be designed for expression of catalytic enzyme proteins in prokaryotic or eukaryotic cells. For example, polypeptides of the invention can be expressed in bacteria (e.g., E. coli), insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector(s) can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. Gene 1988, 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

To maximize recombinant protein expression in E. coli is to express the protein in a host bacterium with an impaired capacity to proteolytically cleave the recombinant protein (Gottesman, S. Gene Expression Technology: Methods in Enzymology 1990 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., Nucleic Acids Res. 1992 20: 2111-2118). Such alteration of nucleic acid sequences of the invention can be carried out by standard DNA synthesis techniques and is described in the Examples.

The catalytic enzyme expression vector can be a yeast expression vector, a vector for expression in insect cells, e.g., a baculovirus expression vector, a vector for expression in bacterial cells, e.g. a plasmid vector, or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In a preferred embodiment, the promoter is an inducible promoter, e.g., a promoter regulated by a steroid hormone, by a polypeptide hormone (e.g., by means of a signal transduction pathway), by a chemical (e.g., Isopropyl β-D-1-thiogalactopyranoside (IPTG)) or by a heterologous polypeptide.

According to an aspect of the present invention, there is provided an isolated nucleic acid molecule encoding at least one catalytic enzyme, or a variant, mutant, or fragment thereof according to any aspect of the present invention. More particularly, the present invention provides an isolated nucleic acid molecule encoding at least one heterologous catalytic enzyme selected from the group comprising:
(a) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(b) an alcohol oxidase or alcohol dehydrogenase for generating an α-hydroxyaldehyde or an α-hydroxyketone from the vicinal diol by an oxidation reaction; and
(c) a transaminase or an amine dehydrogenase for generating a 1,2-aminoalcohol from the α-hydroxyaldehyde or the α-hydroxyketone by a transamination reaction or a reductive amination; or
(d) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(e) an alcohol oxidase or alcohol dehydrogenase for generating an α-hydroxyaldehyde from the vicinal diol by an oxidation reaction;
(f) an aldehyde dehydrogenase or an aldehyde oxidase for generating an α-hydroxy acid from the α-hydroxyaldehyde in an oxidation reaction;
(g) a hydroxy acid dehydrogenase or a hydroxy acid oxidase for generating an α-ketoacid from the α-hydroxy acid in an oxidation reaction; and
(h) a transaminase or an amino acid dehydrogenase for generating an α-amino acid from the α-ketoacid by a transamination reaction or reductive amination reaction.

In a preferred embodiment, the isolated nucleic acid encodes a plurality of catalytic enzymes.

In another preferred embodiment, the isolated nucleic acid encodes a plurality of catalytic enzymes required to transform an alkene starting material to an enantiomerically pure or enantiomerically enriched 1,2-amino alcohol or α-amino acid.

It would be understood that not all of the enzymes introduced into a host cell have to be heterologous; there may be a mixture of heterologous and non-heterologous enzymes depending on the cell type or strain used as host.

In another preferred embodiment, said plurality of catalytic enzymes is arranged as at least one module selected from the group comprising:
i) a module comprising heterologous nucleic acid sequences that, when expressed, enzymatically transforms a terminal alkene to a 1,2-diol;
ii) a module comprising heterologous nucleic acid sequences that, when expressed, enzymatically transforms a 1,2-diol to a 1,2-amino alcohol;
iii) a module comprising heterologous nucleic acid sequences that, when expressed, enzymatically transforms a 1,2-diol to an α-hydroxy acid;
iv) a module comprising heterologous nucleic acid sequences that, when expressed, enzymatically transforms an α-hydroxy acid to an α-amino acid; and
v) a module comprising heterologous nucleic acid sequences that, when expressed, enzymatically transforms an L-amino acid to a terminal alkene.

In another preferred embodiment, said isolated nucleic acid molecule comprises one or more modules selected from the group comprising:
i) a module comprising heterologous nucleic acid sequences that, when expressed, enzymatically transforms a terminal alkene to a 1,2-diol and a module comprising heterologous nucleic acid sequences that, when expressed, enzymatically transforms a 1,2-diol to a 1,2-amino alcohol;
ii) a module comprising heterologous nucleic acid sequences that, when expressed, enzymatically transforms a terminal alkene to a 1,2-diol and a module comprising heterologous nucleic acid sequences that, when expressed, enzymatically transforms a 1,2-diol to an α-hydroxy acid; and
iii) a module comprising heterologous nucleic acid sequences that, when expressed, enzymatically transforms a terminal alkene to a 1,2-diol; a module comprising heterologous nucleic acid sequences that, when expressed, enzymatically transforms a 1,2-diol to a 1,2-amino alcohol and a module comprising heterologous nucleic acid sequences that, when expressed, enzymatically transforms an α-hydroxy acid to an α-amino acid; and

optionally, a module comprising heterologous nucleic acid sequences that, when expressed, enzymatically transforms an L-amino acid to a terminal alkene.

In particular, the isolated nucleic acid molecule may encode:
i) at least one of SEQ ID NOs: 34, 36 and 38, variants, mutants, or fragments thereof to transform a terminal alkene to a 1,2-diol; and/or
ii) at least one of SEQ ID NOs: 2 and 40, variants, mutants, or fragments thereof to transform a 1,2-diol to a 1,2-amino alcohol; and/or
iii) at least one of SEQ ID NOs: 2, 6 and 10, variants, mutants, or fragments thereof to transform a 1,2-diol to an α-hydroxy acid; and/or
iv) at least one of SEQ ID NOs: 16, 20, 24 and 28, variants, mutants, or fragments thereof to transform an α-hydroxy acid to an α-amino acid and/or
v) at least one of SEQ ID NOs: 42, 46 and 50, variants, mutants, or fragments thereof to transform an L-amino acid to a terminal alkene.

More in particular, the isolated nucleic acid molecule may comprise:
i) at least one of SEQ ID NOs: 33, 35 and 37, variants, mutants, or fragments thereof to transform a terminal alkene to a 1,2-diol; and/or
ii) at least one of SEQ ID NOs: 1 and 39, variants, mutants, or fragments thereof to transform a 1,2-diol to a 1,2-amino alcohol; and/or
iii) at least one of SEQ ID NOs: 1, 5 and 9, variants, mutants, or fragments thereof to transform a 1,2-diol to an α-hydroxy acid; and/or
iv) at least one of SEQ ID NOs: 15, 19, 23 and 27, variants, mutants, or fragments thereof to transform an α-hydroxy acid to an α-amino acid; and/or
v) at least one of SEQ ID NOs: 41, 45 and 49, variants, mutants, or fragments thereof to transform an L-amino acid to a terminal alkene.

In a preferred embodiment, the isolated nucleic acid molecule may encode:
i) SEQ ID NOs: 34, 36, 38, 2 and 40, variants, mutants, or fragments thereof to transform a terminal alkene to a 1,2-amino alcohol; and/or
ii) SEQ ID NOs: 34, 36, 38, 2, 6 and 10, variants, mutants, or fragments thereof to transform a terminal alkene to an α-hydroxy acid; and/or
iii) SEQ ID NOs: 34, 36, 38, 2, 40, 16, 20, 24 and 28 variants, mutants, or fragments thereof to transform a terminal alkene to an α-amino acid, and/or
iv) SEQ ID NOs: 42, 46 and 50, variants, mutants, or fragments thereof to transform an L-amino acid to a terminal alkene.

Another aspect the invention provides at least one expression construct comprising at least one nucleic acid sequence that is heterologous according to any aspect of the invention. Preferably the at least one construct comprises a plasmid suitable for expression of at least one catalytic enzyme in a bacterium.

Another aspect the invention provides a host cell which includes at least one nucleic acid molecule described herein, e.g., at least one catalytic enzyme nucleic acid molecule within a recombinant expression vector or a catalytic enzyme nucleic acid molecule containing sequences which allow homologous recombination into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, at least one catalytic enzyme protein can be expressed in bacterial cells (such as E. coli), insect cells (such as Spodoptera frugiperda Sf9 cells), yeast or mammalian cells (such as Chinese hamster ovary cells (CHO) or COS cells (African green monkey kidney cells CV-1 origin SV40 cells; Gluzman Cell 1981, 123: 175-182)). Other suitable host cells are known to those skilled in the art.

One or more vector DNAs can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. For example, according to the invention a host cell may comprise one, two, three, four or more plasmids, each of which may express at least one catalytic enzyme directed to a chemical transformation in the pathway from an alkene starting material to an enantiomerically pure or enantiomerically enriched 1,2-amino alcohol or α-amino acid. The host cell may also include a vector to express catalytic enzymes for providing the alkene by, for example, generating a vinyl carboxylic acid from an α-amino acid by a deamination reaction catalyzed by an ammonia lyase and generating the alkene from the vinyl carboxylic acid in a decarboxylation reaction catalyzed by a decarboxylase.

In a preferred embodiment, the catalytic enzymes required to transform an alkene starting material to an enantiomerically pure or enantiomerically enriched 1,2-amino alcohol or α-amino acid are arranged on expression vectors as modules, wherein each module comprises a combination of catalytic enzymes to perform specific reactions within the overall system. For example, a first module may comprise enzymes for the dihydroxylation of a terminal alkene to a 1,2-diol co-expressed on a plasmid; a second module may comprise enzymes for the oxidation-amination of a 1,2-diol to a 1,2-amino alcohol co-expressed on a plasmid; a third module may comprise enzymes for the double oxidation of a 1,2-diol to an α-hydroxy acid co-expressed on a plasmid; a fourth module may comprise enzymes for the oxidation-amination of α-hydroxy acid to α-amino acid co-expressed on a plasmid; and a fifth module may comprise enzymes for the deamination-decarboxylation of an α-amino acid to an alkene. Arrangements of such enzymes as modules allow flexibility in constructing a serial cascade of reactions in one pot. One or more modules may be engineered onto the same plasmid. For example, a host cell comprising the said first and third modules, on the same or separate plasmids, is capable of catalyzing the conversion of a terminal alkene to a 1,2-amino alcohol. Likewise a host cell comprising said first, a second and fourth module is capable of catalyzing the conversion of a terminal alkene to an α-amino acid. If module 5 is co-expressed in the host cell, an α-amino acid feed stock can be provided for the cell to generate its own terminal alkene for the cascade reactions.

In one aspect of the invention there is provided one or more recombinant prokaryotic or eukaryotic cells selected from the group comprising bacterial cells, yeast cells, mammalian cells and insect cells, wherein said cells comprise at least one expression construct and/or heterologous nucleic acid molecule that encodes at least one catalytic enzyme required in the pathway from alkene to enantiomerically pure or enantiomerically enriched 1,2-amino alcohol or from an α-amino acid to enantiomerically pure or enantiomerically enriched 1,2-amino alcohol.

The cells may contain a single expression vector or construct, such as a plasmid, which expresses a single catalytic enzyme or co-expresses a plurality of catalytic enzymes as described herein under the control of at least one regulatory element. The catalytic enzymes may be arranged in the plasmid as an individual artificial operon under the control of a promoter with one ribosome-binding site before every gene, or arranged with individual promoters. Accordingly, a one-pot synthesis cascade may be achieved with cells expressing all required catalytic enzymes on one or several plasmids, or with different recombinant cells which each express a specific repertoire of catalytic enzymes, providing the necessary cells are included for a particular chemical transformation.

In a preferred embodiment, the cells are recombinant bacterial cells; more preferably E. coli cells.

In another preferred embodiment, said cells comprise at least one expression construct selected from the group comprising
i) a module comprising heterologous nucleic acid sequences that, when expressed, enzymatically transforms a terminal alkene to a 1,2-diol;
ii) a module comprising heterologous nucleic acid sequences that, when expressed, enzymatically transforms a 1,2-diol to a 1,2-amino alcohol;
iii) a module comprising heterologous nucleic acid sequences that, when expressed, enzymatically transforms a 1,2-diol to an α-hydroxy acid;
iv) a module comprising heterologous nucleic acid sequences that, when expressed, enzymatically transforms an α-hydroxy acid to an α-amino acid; and
v) a module comprising heterologous nucleic acid sequences that, when expressed, enzymatically transforms an L-amino acid to a terminal alkene.

In another preferred embodiment, said catalytic enzymes for use in the invention have at least 60% 65%, 70%, 75%, 80%, 85%, 90%, 95%, 96%, 97%, 98%, 99%, or more, homology or amino acid identity with at least one enzyme selected from the group comprising an alcohol dehydrogenase with amino acid sequence represented by SEQ ID NO: 2, an ω-transaminase with amino acid sequence represented by SEQ ID NO: 5, an alanine dehydrogenase with amino acid sequence represented by SEQ ID NO: 9, a styrene monooxygenase with amino acid sequence represented by SEQ ID NOs: 34 & 36, an epoxide hydrolase with amino acid sequence represented by SEQ ID NO: 38, an aldehyde dehydrogenase with amino acid sequence represented by SEQ ID NO: 40; a phenylacrylic acid decarboxylase with amino acid sequence represented by SEQ ID NOs: 42 & 45 and a phenylalanine ammonia lyase with amino acid sequence represented by SEQ ID NO: 50.

In another preferred embodiment, the one or more recombinant cells express catalytic enzymes selected from the groups comprising;
i) styrene monooxygenase, epoxide hydrolase, alcohol dehydrogenase, ω-transaminase, and alanine dehydrogenase, variants or bioactive fragments thereof for producing a 1, 2 amino-alcohol from a terminal alkene; or
ii) styrene monooxygenase, epoxide hydrolase, alcohol dehydrogenase, aldehyde dehydrogenase, hydroxy acid oxidase, α-transaminase, glutamate dehydrogenase, and catalase, variants or bioactive fragments thereof for producing an alpha amino acid from a terminal alkene and, optionally;
iii) lyase and decarboxylase, variants or bioactive fragments thereof for producing a terminal alkene from an L-amino acid.

In a preferred embodiment, in iii) the lyase is a phenylalanine ammonia lyase (PAL) and the decarboxylase is a phenylacrylic acid decarboxylase (PAD).

A host cell of the invention can be used to produce (i.e., express) one or more catalytic enzyme proteins. Accordingly, the invention further provides methods for producing one or more catalytic enzyme proteins, e.g., one or more catalytic enzyme proteins described herein, using the host cells of the invention. In one embodiment, the method includes culturing the host cell of the invention (into which one or more recombinant expression vector(s) encoding one or more catalytic enzyme proteins has/have been introduced) in a suitable medium such that one or more catalytic enzyme proteins is/are produced. In another embodiment, the method further includes isolating one or more catalytic enzyme proteins from the medium or the host cell.

According to another aspect of the invention there is provided a kit comprising at least one recombinant cell, expression construct or isolated nucleic acid according to any aspect of the invention. Preferably, the kit can be used to provide one or more components for a one-pot system to enzyme-catalyze the production of an enantiomerically pure or enantiomerically enriched 1,2-amino alcohol or α-amino acid from an alkene starting material.

### EXAMPLES

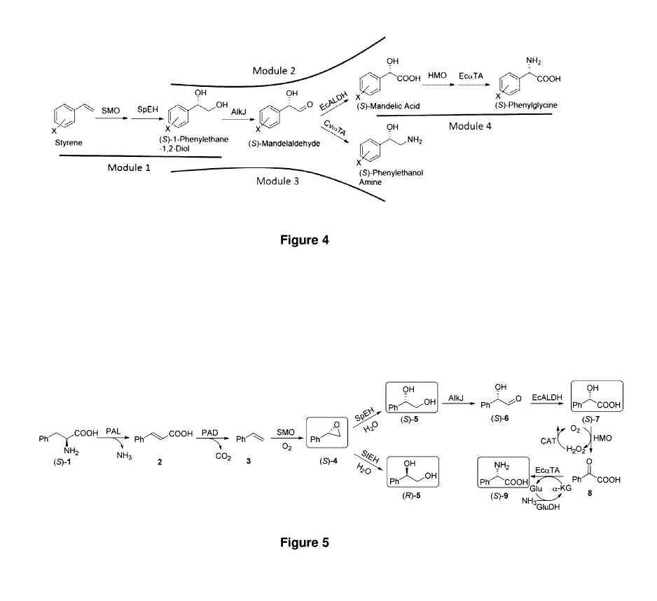

One-pot region and stereoselective multiple oxy- and amino-functionalizations of terminal alkenes to produce chiral 1,2-amino alcohols and α-amino acids, respectively, are designed as the target reactions. To realize the targeted asymmetric alkene functionalizations, microbial cells containing two to three basic enzyme modules were designed, with each of them catalyzing two to four enzymatic reactions (FIG. 4). The basic modules were designed by using the following criteria: (a) each module utilizes a stable input, such as alkene, diol and hydroxy acid, and gives a stable output, such as diol, hydroxyl acid, amino alcohol and amino acid; (b) each module enables fast conversion of unstable or toxic intermediates, such as epoxide, hydroxy aldehyde and keto acid, to minimize their accumulation and side reactions. Assemblies of module 1 and 3 in one cell and module 1, 2 and 4 in another cell gave rise to whole-cell catalysts for one-pot transformations of terminal alkene to chiral 1,2-amino alcohol and α-amino acid, respectively. To demonstrate the concept, biotransformations of styrenes and derivatives to (S)-1,2-amino alcohols and (S)-α-amino acids were chosen as the representative examples of the three types of asymmetric reactions (FIG. 4). While the styrenes are easily available substrates, (S)-1,2-amino alcohols and (S)-α-amino acids are highly valuable chiral chemicals with many applications.

Besides using alkenes as the starting material, the use of readily available amino acids is an attractive alternative as they are currently produced by fermentation in large amounts and at low cost. With that in mind, the one-pot biotransformation of biobased L-phenylalanine to valuable chiral compounds via cascade biocatalysis was achieved as an example (FIG. 5). Specifically, the biocatalytic cascade first involves the deamination-decarboxylation of L-phenylalanine (S)-1 to styrene (S)-3 by using phenylalanine ammonia lyase (PAL) and phenylacrylic acid decarboxylase (PAD). This is followed by the combination of the different enzyme modules for the functionalization of the alkenes to produce various chiral compounds (S)-5, (R)-5, (S)-6, (S)-7, (S)-8 and (S)-9. All of these can be incorporated into a single E. coli strain for the enantioselective conversion of L-phenylalanine to chiral epoxide, diols, hydroxyl acid, and amino acid, respectively, with no chemical counterparts, via cascade biocatalysis consisting of 3-8 enzymatic steps.

Strain, Biochemicals, and Culture Medium

Escherichia coli T7 expression cells were purchased from New England Biolabs. Primers (DNA oligos) were synthesized from IDT. Phusion DNA polymerase, fast digest restriction enzymes, and T4 DNA ligase were bought from Thermo Scientific. LB medium, tryptone, yeast extract, and agar were obtained from Biomed Diagnostics. Chloramphenicol, streptomycin, ampicillin, kanamycin, and glucose were purchased from Sigma-Aldrich. IPTG (Isopropyl β-D-1-thiogalactopyranoside) was obtained from Gold Biotechnology.

The culture medium used in this study is standard M9 medium supplemented with glucose (20 g/L), yeast extract (6 g/L). The M9 medium contains 6 g/L Na₂HPO₄, 3.0 g/L KH₂PO₄, 0.5 g/L NaCl, 1.0 g/L NH₄Cl, 1 mM MgSO₄, 0.1 mM CaCl₂, and 1 mL/L l⁻¹ trace metal solution. The trace metal solution contains 8.3 g/L FeCl₃.6H₂O, 0.84 g/L ZnCl₂, 0.13 g/L CuCl₂.2H₂O, 0.1 g/L CoCl₂.2H₂O, 0.1 g/L H₃BO₃, 0.016 g/L MnCl₂.4H₂O, and 0.1 g/L Na₂MoO₄.2H₂O in 1 M HCl.

SDS-PAGE Analysis and Quantification

Freshly prepared E. coli whole cells were centrifuged and resuspended in DI water to a density of 8 g cdw/L (OD₆₀₀=20). The cell suspension (60 μL) was mixed with 20 μl of SDS sample buffer (4× Laemmli Sample Buffer with DTT, Bio-Rad) and heated to 98° C. for 15 min. 60 μl of 0.2 g/L, 0.1 g/L, and 0.05 g/L of BSA standards were also mixed with 20 μL of SDS sample buffer and heated to 98° C. for 15 min. Then the mixture was centrifuged (13000 g) for 10 min. 10 μL of the supernatant was used to load into the sample well of 12% SDS-PAGE gel (hand cast). The electrophoresis was run in a setup of Mini-Protean tetra cell at 100 V for 15 min and 150 V for 75 min. After running, the PAGE gel was washed with water and then stained with Bio-Safe Coomassie Stain (Bio-Rad) according to the instruction. The figure was obtained with GS-900 calibrated densitometer (Bio-Rad), and quantification analysis was done with the volume tools in the Image Lab software (Bio-Rad).

General Procedure 1: Genetic Engineering of Recombinant E. coli Strains

Escherichia coli T7 expression strain (an E. coli B strain derivative) was purchased from New England Biolabs (#C25661) and used as host strain for all molecular cloning and biocatalysis experiments. The gene module 1 comprising of styA, styB and spEH was constructed previously (Wu, S. et al. ACS Catal. 2014, 4: 409-420). AlkJ gene was amplified from the OCT megaplasmid extracted from P. putida GPo1 as reported (Kirmair, L. & Skerra, A. Appl. Environ. Microbiol. 2014, 80: 2468-2477). Genes of padA, ilvE, gdhA and katE were amplified from the genomic DNA extracted from E. coli K12 MG 1655 with genomic DNA Purification Kit (Thermo Scientific). Ald gene was amplified from the genomic DNA extracted from B. subtilis str.168 with genomic DNA Purification Kit. Codon-optimized cv 2025 gene was synthesized from Genscript based on the sequence from C. violaceum DSM30191 (Kaulmann, U. et al., Enzyme Microb. Technol. 2007, 41: 628-637). Codon-optimized sco3228 gene was synthesized from Genscript based on the sequence from S. coelicolor A3(2) (Li, T. L. et al. Chem. Commun. 2001, 18: 1752-1753)

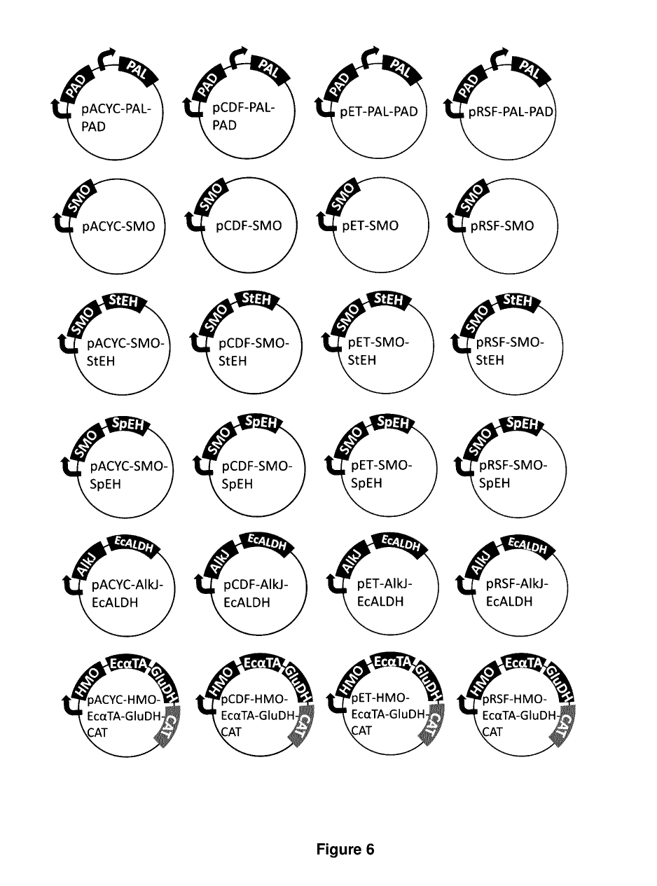

All genetic constructions were carried out by using standard molecular biology techniques with Phusion DNA polymerase, FastDigest restriction enzymes and T4 DNA ligase (all from Thermo Scientific). PCR primers were synthesized from Integrated DNA Technologies. Purification of DNA after electrophoresis or enzyme digestion was performed with E.Z.N.A. Gel Extraction Kit (Omega Biotek), and extraction of plasmids was performed with Axyprep Plasmid Miniprep Kit (Axygen). Basic gene modules 1-4 were constructed on a set of compatible plasmids pACYCDuet-1, pCDFDuet-1, pETDuet-1 and pRSFDuet-1 (Novagen) as individual artificial operon under control of a T7 promoter with one ribosome-binding site before every gene. Gene modules were transformed into E. coli T7 competent cells to obtain the E. coli with individual basic modules. Further transformation of other basic genetic module(s) into a constructed E. coli strain containing one or two basic modules gave an E. coli strain containing two or three basic modules for the desired asymmetric alkene functionalization reactions. Recombinant E. coli strains generated in the Examples are shown in Table 1 and the recombinant plasmids used in the study are shown in FIG. 6.

TABLE 1
List of recombinant strains, the plasmids contained in the strains, and
the cascade biotransformation catalyzed by the strains.

| | | Cascade |
|---|---|---|
| Strain^{[a]} | Recombinant plasmids^{[b]} in the strain | Reactions |
| E. coli (LZ01) | pACYC-PAL-PAD, pCDF-SMO | (S)-1 to (S)-4 |
| E. coli (LZ02) | pACYC-PAL-PAD, pET-SMO | (S)-1 to (S)-4 |
| E. coli (LZ03) | pACYC-PAL-PAD, pRSF-SMO | (S)-1 to (S)-4 |
| E. coli (LZ04) | pCDF-PAL-PAD, pACYC-SMO | (S)-1 to (S)-4 |
| E. coli (LZ05) | pCDF-PAL-PAD, pET-SMO | (S)-1 to (S)-4 |
| E. coli (LZ06) | pCDF-PAL-PAD, pRSF-SMO | (S)-1 to (S)-4 |
| E. coli (LZ07) | pET-PAL-PAD, pACYC-SMO | (S)-1 to (S)-4 |
| E. coli (LZ08) | pET-PAL-PAD, pCDF-SMO | (S)-1 to (S)-4 |
| E. coli (LZ09) | pET-PAL-PAD, pRSF-SMO | (S)-1 to (S)-4 |
| E. coli (LZ10) | pRSF-PAL-PAD, pACYC-SMO | (S)-1 to (S)-4 |
| E. coli (LZ1 1) | pRSF-PAL-PAD, pCDF-SMO | (S)-1 to (S)-4 |
| E. coli (LZ12) | pRSF-PAL-PAD, pET-SMO | (S)-1 to (S)-4 |
| E. coli (LZ13) | pACYC-PAL-PAD, pCDF-SMO-StEH | (S)-1 to (R)-5 |
| E. coli (LZ14) | pACYC-PAL-PAD, pET-SMO-StEH | (S)-1 to (R)-5 |
| E. coli (LZ15) | pACYC-PAL-PAD, pRSF-SMO-StEH | (S)-1 to (R)-5 |
| E. coli (LZ16) | pCDF-PAL-PAD, pACYC-SMO-StEH | (S)-1 to (R)-5 |
| E. coli (LZ17) | pCDF-PAL-PAD, pET-SMO-StEH | (S)-1 to (R)-5 |
| E. coli (LZ18) | pCDF-PAL-PAD, pRSF-SMO-StEH | (S)-1 to (R)-5 |
| E. coli (LZ19) | pET-PAL-PAD, pACYC-SMO-StEH | (S)-1 to (R)-5 |
| E. coli (LZ20) | pET-PAL-PAD, pCDF-SMO-StEH | (S)-1 to (R)-5 |
| E. coli (LZ21) | pET-PAL-PAD, pRSF-SMO-StEH | (S)-1 to (R)-5 |
| E. coli (LZ22) | pRSF-PAL-PAD, pACYC-SMO-StEH | (S)-1 to (R)-5 |
| E. coli (LZ23) | pRSF-PAL-PAD, pCDF-SMO-StEH | (S)-1 to (R)-5 |
| E. coli (LZ24) | pRSF-PAL-PAD, pET-SMO-StEH | (S)-1 to (R)-5 |
| E. coli (LZ25) | pACYC-PAL-PAD, pCDF-SMO-SpEH | (S)-1 to (S)-5 |
| E. coli (LZ26) | pACYC-PAL-PAD, pET-SMO-SpEH | (S)-1 to (S)-5 |
| E. coli (LZ27) | pACYC-PAL-PAD, pRSF-SMO-SpEH | (S)-1 to (S)-5 |
| E. coli (LZ28) | pCDF-PAL-PAD, pACYC-SMO-SpEH | (S)-1 to (S)-5 |
| E. coli (LZ29) | pCDF-PAL-PAD, pET-SMO-SpEH | (S)-1 to (S)-5 |
| E. coli (LZ30) | pCDF-PAL-PAD, pRSF-SMO-SpEH | (S)-1 to (S)-5 |
| E. coli (LZ31) | pET-PAL-PAD, pACYC-SMO-SpEH | (S)-1 to (S)-5 |
| E. coli (LZ32) | pET-PAL-PAD, pCDF-SMO-SpEH | (S)-1 to (S)-5 |
| E. coli (LZ33) | pET-PAL-PAD, pRSF-SMO-SpEH | (S)-1 to (S)-5 |
| E. coli (LZ34) | pRSF-PAL-PAD, pACYC-SMO-SpEH | (S)-1 to (S)-5 |
| E. coli (LZ35) | pRSF-PAL-PAD, pCDF-SMO-SpEH | (S)-1 to (S)-5 |
| E. coli (LZ36) | pRSF-PAL-PAD, pET-SMO-SpEH | (S)-1 to (S)-5 |
| E. coli (LZ37) | pACYC-PAL-PAD, pCDF-SMO-SpEH, | (S)-1 to (S)-7 |
| | pET-AlkJ-EcALDH | |
| E. coli (LZ38) | pACYC-PAL-PAD, pCDF-SMO-SpEH, | (S)-1 to (S)-7 |
| | pRSF-AlkJ-EcALDH | |
| E. coli (LZ39) | pACYC-PAL-PAD, pET-SMO-SpEH, | (S)-1 to (S)-7 |
| | pCDF-AlkJ-EcALDH | |
| E. coli (LZ40) | pACYC-PAL-PAD, pET-SMO-SpEH, | (S)-1 to (S)-7 |
| | pRSF-AlkJ-EcALDH | |
| E. coli (LZ41) | pACYC-PAL-PAD, pRSF-SMO-SpEH, | (S)-1 to (S)-7 |
| | pCDF-AlkJ-EcALDH | |
| E. coli (LZ42) | pACYC-PAL-PAD, pRSF-SMO-SpEH, | (S)-1 to (S)-7 |
| | pET-AlkJ-EcALDH | |
| E. coli (LZ43) | pCDF-PAL-PAD, pACYC-SMO-SpEH, | (S)-1 to (S)-7 |
| | pET-AlkJ-EcALDH | |
| E. coli (LZ44) | pCDF-PAL-PAD, pACYC-SMO-SpEH, | (S)-1 to (S)-7 |
| | pRSF-AlkJ-EcALDH | |
| E. coli (LZ45) | pCDF-PAL-PAD, pET-SMO-SpEH, | (S)-1 to (S)-7 |
| | pACYC-AlkJ-EcALDH | |
| E. coli (LZ46) | pCDF-PAL-PAD, pET-SMO-SpEH, | (S)-1 to (S)-7 |
| | pRSF-AlkJ-EcALDH | |
| E. coli (LZ47) | pCDF-PAL-PAD, pRSF-SMO-SpEH, | (S)-1 to (S)-7 |
| | pACYC-AlkJ-EcALDH | |
| E. coli (LZ48) | pCDF-PAL-PAD, pRSF-SMO-SpEH, | (S)-1 to (S)-7 |
| | pET-AlkJ-EcALDH | |
| E. coli (LZ49) | pET-PAL-PAD, pACYC-SMO-SpEH, | (S)-1 to (S)-7 |
| | pCDF-AlkJ-EcALDH | |
| E. coli (LZ50) | pET-PAL-PAD, pACYC-SMO-SpEH, | (S)-1 to (S)-7 |
| | pRSF-AlkJ-EcALDH | |
| E. coli (LZ51) | pET-PAL-PAD, pCDF-SMO-SpEH, | (S)-1 to (S)-7 |
| | pACYC-AlkJ-EcALDH | |
| E. coli (LZ52) | pET-PAL-PAD, pCDF-SMO-SpEH, | (S)-1 to (S)-7 |
| | pRSF-AlkJ-EcALDH | |
| E. coli (LZ53) | pET-PAL-PAD, pRSF-SMO-SpEH, | (S)-1 to (S)-7 |
| | pACYC-AlkJ-EcALDH | |
| E. coli (LZ54) | pET-PAL-PAD, pRSF-SMO-SpEH, | (S)-1 to (S)-7 |
| | pCDF-AlkJ-EcALDH | |
| E. coli (LZ55) | pRSF-PAL-PAD, pACYC-SMO-SpEH, | (S)-1 to (S)-7 |
| | pCDF-AlkJ-EcALDH | |
| E. coli (LZ56) | pRSF-PAL-PAD, pACYC-SMO-SpEH, | (S)-1 to (S)-7 |
| | pET-AlkJ-EcALDH | |
| E. coli (LZ57) | pRSF-PAL-PAD, pCDF-SMO-SpEH, | (S)-1 to (S)-7 |
| | pACYC-AlkJ-EcALDH | |
| E. coli (LZ58) | pRSF-PAL-PAD, pCDF-SMO-SpEH, | (S)-1 to (S)-7 |
| | pET-AlkJ-EcALDH | |
| E. coli (LZ59) | pRSF-PAL-PAD, pET-SMO-SpEH, | (S)-1 to (S)-7 |
| | pACYC-AlkJ-EcALDH | |
| E. coli (LZ60) | pRSF-PAL-PAD, pET-SMO-SpEH, | (S)-1 to (S)-7 |
| | pCDF-AlkJ-EcALDH | |
| E. coli (LZ61) | pACYC-PAL-PAD, pCDF-SMO-SpEH, | (S)-1 to (S)-9 |
| | pET-AlkJ-EcALDH, pRSF-HMO- | |
| | EcaTA-GluDH-CAT | |
| E. coli (LZ62) | pACYC-PAL-PAD, pCDF-SMO-SpEH, | (S)-1 to (S)-9 |
| | pRSF-AlkJ-EcALDH, pET-HMO- | |
| | EcaTA-GluDH-CAT | |
| E. coli (LZ63) | pACYC-PAL-PAD, pET-SMO-SpEH, | (S)-1 to (S)-9 |
| | pCDF-AlkJ-EcALDH, pRSF-HMO- | |
| | EcaTA-GluDH-CAT | |
| E. coli (LZ64) | pACYC-PAL-PAD, pET-SMO-SpEH, | (S)-1 to (S)-9 |
| | pRSF-AlkJ-EcALDH, pET-HMO-EcaTA- | |
| | GluDH-CAT | |
| E. coli (LZ65) | pACYC-PAL-PAD, pRSF-SMO-SpEH, | (S)-1 to (S)-9 |
| | pCDF-AlkJ-EcALDH, pET-HMO-EcaTA- | |
| | GluDH-CAT | |
| E. coli (LZ66) | pACYC-PAL-PAD, pRSF-SMO-SpEH, | (S)-1 to (S)-9 |
| | pET-AlkJ-EcALDH, pCDF-HMO-EcaTA- | |
| | GluDH-CAT | |
| E. coli (LZ67) | pCDF-PAL-PAD, pACYC-SMO-SpEH, | (S)-1 to (S)-9 |
| | pET-AlkJ-EcALDH, pRSF-HMO-EcaTA- | |
| | GluDH-CAT | |
| E. coli (LZ68) | pCDF-PAL-PAD, pACYC-SMO-SpEH, | (S)-1 to (S)-9 |
| | pRSF-AlkJ-EcALDH, pET-HMO-EcaTA- | |
| | GluDH-CAT | |
| E. coli (LZ69) | pCDF-PAL-PAD, pET-SMO-SpEH, | (S)-1 to (S)-9 |
| | pACYC-AlkJ-EcALDH, pRSF-HMO- | |
| | EcaTA-GluDH-CAT | |
| E. coli (LZ70) | pCDF-PAL-PAD, pET-SMO-SpEH, | (S)-1 to (S)-9 |
| | pRSF-AlkJ-EcALDH, pACYC-HMO- | |
| | EcaTA-GluDH-CAT | |
| E. coli (LZ71) | pCDF-PAL-PAD, pRSF-SMO-SpEH, | (S)-1 to (S)-9 |
| | pACYC-AlkJ-EcALDH, pET-HMO- | |
| | EcaTA-GluDH-CAT | |
| E. coli (LZ72) | pCDF-PAL-PAD, pRSF-SMO-SpEH, | (S)-1 to (S)-9 |
| | pET-AlkJ-EcALDH, pACYC-HMO- | |
| | EcaTA-GluDH-CAT | |
| E. coli (LZ73) | pET-PAL-PAD, pACYC-SMO-SpEH, | (S)-1 to (S)-9 |
| | pCDF-AlkJ-EcALDH, pRSF-HMO- | |
| | EcaTA-GluDH-CAT | |
| E. coli (LZ74) | pET-PAL-PAD, pACYC-SMO-SpEH, | (S)-1 to (S)-9 |
| | pRSF-AlkJ-EcALDH, pCDF-HMO- | |
| | EcaTA-GluDH-CAT | |
| E. coli (LZ75) | pET-PAL-PAD, pCDF-SMO-SpEH, | (S)-1 to (S)-9 |
| | pACYC-AlkJ-EcALDH, pRSF-HMO- | |
| | EcaTA-GluDH-CAT | |
| E. coli (LZ76) | pET-PAL-PAD, pCDF-SMO-SpEH, | (S)-1 to (S)-9 |
| | pRSF-AlkJ-EcALDH, pACYC-HMO- | |
| | EcaTA-GluDH-CAT | |
| E. coli (LZ77) | pET-PAL-PAD, pRSF-SMO-SpEH, | (S)-1 to (S)-9 |
| | pACYC-AlkJ-EcALDH, pCDF-HMO- | |
| | EcaTA-GluDH-CAT | |
| E. coli (LZ78) | pET-PAL-PAD, pRSF-SMO-SpEH, | (S)-1 to (S)-9 |
| | pCDF-AlkJ-EcALDH, pACYC-HMO- | |
| | EcaTA-GluDH-CAT | |
| E. coli (LZ79) | pRSF-PAL-PAD, pACYC-SMO-SpEH, | (S)-1 to (S)-9 |
| | pCDF-AlkJ-EcALDH, pET-HMO- | |
| | EcaTA-GluDH-CAT | |
| E. coli (LZ80) | pRSF-PAL-PAD, pACYC-SMO-SpEH, | (S)-1 to (S)-9 |
| | pET-AlkJ-EcALDH, pCDF-HMO- | |
| | EcaTA-GluDH-CAT | |
| E. coli (LZ81) | pRSF-PAL-PAD, pCDF-SMO-SpEH, | (S)-1 to (S)-9 |
| | pACYC-AlkJ-EcALDH, pET-HMO- | |
| | EcaTA-GluDH-CAT | |
| E. coli (LZ82) | pRSF-PAL-PAD, pCDF-SMO-SpEH, | (S)-1 to (S)-9 |
| | pET-AlkJ-EcALDH, pACYC-HMO- | |
| | EcaTA-GluDH-CAT | |
| E. coli (LZ83) | pRSF-PAL-PAD, pET-SMO-SpEH, | (S)-1 to (S)-9 |
| | pACYC-AlkJ-EcALDH, pCDF-HMO- | |
| | EcaTA-GluDH-CAT | |
| E. coli (LZ84) | pRSF-PAL-PAD, pET-SMO-SpEH, | (S)-1 to (S)-9 |
| | pCDF-AlkJ-EcALDH, pACYC-HMO- | |
| | EcaTA-GluDH-CAT | |

Module 1:

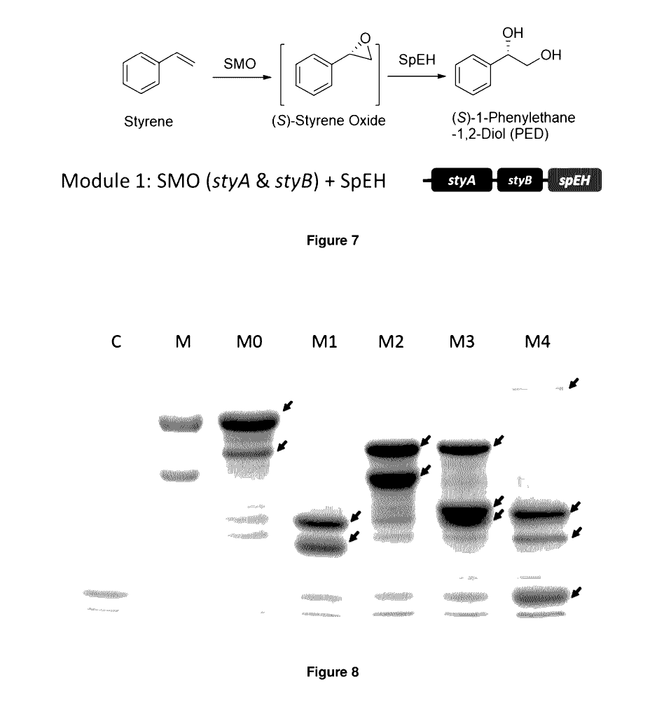

A representative example of an enzyme catalytic cascade is the conversion of (substituted) styrene to chiral (substituted) (S)-phenylethanol amine and (S)-phenylglycine (FIG. 4). Module 1 effects the transformation of styrene to (S)-phenylethane diol (PED) (FIG. 7). Previously, we engineered E. coli co-expressing styrene monooxygenase (SMO, from styrene degradation Pseudomonas sp. VLB120) and an epoxide hydrolase (SpEH) from Sphingomonas sp. HXN-200 to do the same chemistry and to produce (S)-PEDs in high e.e. and in good yield, without accumulation of epoxides (Wu, S. et al. ACS Catal. 2014, 4: 409-420). Thus, the genetic construction of an artificial operon containing styA, styB, and spEH was used as Module 1 in this work (FIG. 7). The expression of two enzymes was examined by SDS-PAGE analysis, and both StyA and SpEH were clearly visible (FIG. 8, lane M1). Module 1 was sub-cloned into four different but compatible plasmids (pACYCDuet, pCDFDuet, pETDuet, and pRSFDuet) to give A-M1, C-M1, E-M1, and R-M1, respectively.

Module 2

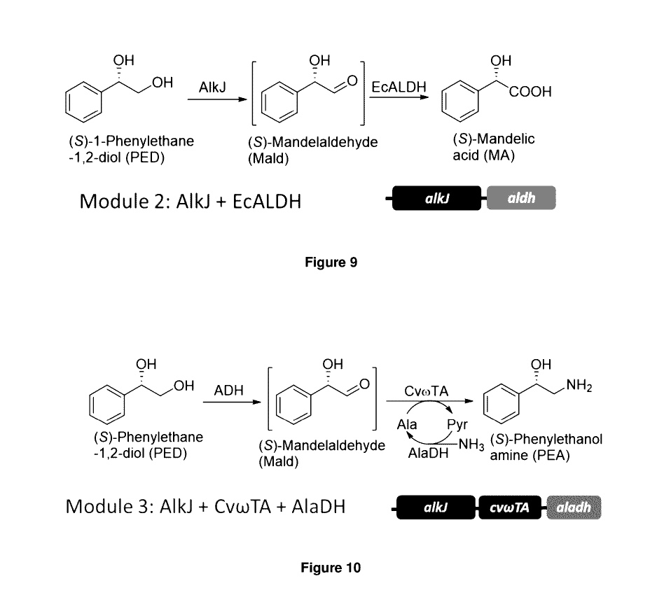

Module 2 effects the terminal oxidation of (S)-phenylethane diol to (S)-mandelic acid by alcohol dehydrogenase and aldehyde dehydrogenase (FIG. 9). Previously, we engineered E. coli to co-express alkanol dehydrogenase (AlkJ, from alkane degradation Pseudomonas putida GPo1) and aldehyde dehydrogenase (EcALDH) from E. coli (Li, Z. et al. PCT Application No. PCT/SG2014/000221). The recombinant E. coli expressing these two enzymes converted (S)-phenylethane diol to (S)-mandelic acid efficiently. Hence, the genetic construction of AlkJ and EcALDH was directly used here as Module 2, which was also sub-cloned to four plasmids to give A-M2, C-M2, E-M2, and R-M2, similarly.

General Procedure 2: Growing of E. coli Strains

Recombinant E. coli strain was first inoculated in 1 mL LB medium containing appropriate antibiotics (50 mg/L chloramphenicol, 50 mg/L streptomycin, 100 mg/L ampicillin, 50 mg/L kanamycin or a combination of them) at 37° C. for 7-10 h. The culture was then transferred into 25 mL M9 medium containing glucose (20 g/L), yeast extract (6 g/L) and appropriate antibiotics in a 125 mL tri-baffled flask. The cells were grown at 37° C. and 300 r.p.m. for about 2 h to reach an OD₆₀₀ of 0.6, followed by the addition of IPTG (0.5 mM) to induce the enzyme expression. The cells were grown for 12-13 h at 22° C. to reach late exponential phase, and they were collected by centrifugation (3500 g, 10 min). The cell pellets were resuspended in an appropriate buffer to the desired density as resting cells for biotransformation.

General Procedure 3: Conversion of Substituted Styrenes to Substituted (S)-Phenylethanol Amines Using E. coli Containing Module 1 and 3

Overall, 2 mL suspension (10 g cdw/L) of freshly prepared E. coli (AE) cells in NaP buffer (200 mM, pH 8.0) containing glucose (1%, w/v) and NH₃/NH₄Cl (200 mM, NH₃:NH₄Cl=1:10) were mixed with 2 ml n-hexadecane containing one of the substituted styrene substrates (20 mM). The mixture was shaken at 300 r.p.m. and 25° C. for 24 h. At 12 h, additional glucose (0.5%, w/v) and NH₃/NH₄Cl (100 mM) were added. Aliquots of each phase of the samples (150 μL) were taken out at different time points and prepared for analysis. For organic phase, 100 μL n-hexadecane was separated after centrifugation (13000 g, 2 min), diluted with 900 μL n-hexane (containing 2 mM benzyl alcohol as internal standard) and analyzed by normal phase HPLC for quantifying the substituted styrenes and possible epoxides. For aqueous phase, 100 μL supernatant were separated after centrifugation (13000 g, 2 min), diluted with 400 μL TFA solution (0.5%) and 500 μL acetonitrile (containing 2 mM benzyl alcohol as internal standard) and then analyzed by reverse phase HPLC for quantifying the substituted (S)-phenylethanol amines. To determine the e.e. of the substituted (S)-phenylethanol amines, the remaining aqueous phase at the end of reaction was separated after centrifugation (13000 g, 2 min), acidified with TFA, and 100 μL of the sample was separated and diluted with 900 μL TFA solution (0.1%) for chiral HPLC analysis.

General Procedure 4: Conversion of Substituted Styrenes to Substituted (S)-Phenylglycines Using E. coli Containing Module 1, 2, and 4

Overall, 2 ml suspension (10 g cdw/L) of freshly prepared E. coli (ARC) cells in KP buffer (200 mM, pH 8.0) containing glucose (0.5%, w/v) and NH₃/NH₄Cl (100 mM, NH₃:NH₄Cl=1:10) were mixed with 2 mL n-hexadecane containing one of the substituted styrene substrates (20 or 5 mM). The reaction mixture was shaken at 300 r.p.m. and 30° C. for 24 h. At 20 h, additional glucose (0.5%, w/v) and NH₃/NH₄Cl (100 mM) were added. 300 μL aliquots of the mixture (150 μL of each phase) were taken out at different time points for following the reaction. 150 μL HCl solution (0.8 M) were mixed with the 300 μL sample, followed by centrifugation (13000 g, 2 min) to separate the organic and aqueous phases. For organic phase, 100 μL n-hexadecane was separated after centrifugation (13000 g, 2 min), diluted with 900 μL n-hexane (containing 2 mM benzyl alcohol as internal standard) and analyzed by normal phase HPLC for quantifying the substituted styrenes and possible epoxides. For the aqueous phase, 200 μL supernatant were diluted with 300 μL TFA solution (0.1%) and 500 μL acetonitrile (containing 2 mM benzyl alcohol as internal standard), and the samples were analyzed by reverse phase HPLC for quantifying the substituted (S)-phenylglycines and other hydrophilic products. To determine the e.e. of the substituted (S)-phenylglycines, the remaining aqueous phase at the end of reaction was separated after centrifugation (13000 g, 2 min), acidified with TFA, and 100 μL of the sample was separated and diluted with 900 μL TFA solution (0.1%) for chiral HPLC analysis.

### Example 1

### Genetic Engineering of E. coli Containing Module 3 and Expressing AlkJ, CvωTA, and AlaDH

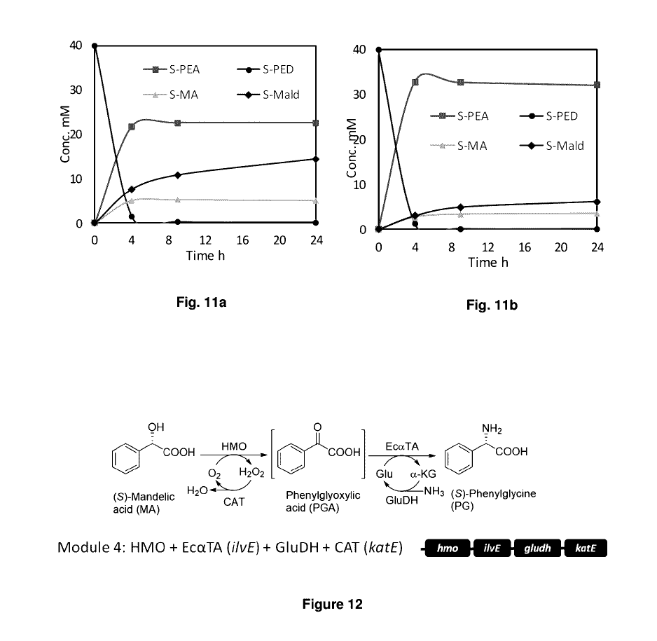

Briefly, Module 3 is a cascade transformation, which includes oxidation of terminal alcohol to aldehyde and reductive amination of aldehyde to amine. As AlkJ catalyzed the highly regioselective oxidation of (S)-phenylethane diol to (S)-mandelaldehyde in Module 2, it is also used as the first enzyme in Module 3. For the second step (reductive amination), we cloned and tested the ω-transaminase (ω-TA) from Chromobacterium violaceum (CvωTA), which had been reported to catalyzed a very broad substrate scope. An E. coli strain was engineered to co-express AlkJ and CvωTA, and biotransformation of 40 mM (S)-phenylethane diol using 200 mM L-alanine as amine donor gave 22 mM desired (S)-phenylethanol amine (55% yield), with 13 mM (S)-mandelaldehyde (intermediate) and 5 mM (S)-mandelic acid (byproduct) remained in the system (FIG. 11a). To increase the yield of amine and utilize the easily available ammonia as amine donor, we employed L-alanine dehydrogenase (AlaDH) from Bacillus subtilis to regenerate L-alanine from pyruvate using ammonia. An E. coli strain was engineered to co-express AlkJ, CvωTA, and AlaDH by construction of a non-natural operon (Module 3, FIG. 10, protein expression see FIG. 8, lane M3). By using this strain, the biotransformation of 40 mM (S)-phenylethane diol was significantly improved to afford 32 mM (S)-phenylethanol amine (80% yield) with 200 mM ammonia without external L-alanine (FIG. 11b). Module 3 was also sub-cloned to four plasmids to give A-M3, C-M3, E-M3, and R-M3, similarly.

More particularly, the alkJ gene nucleic acid sequence (SEQ ID NO: 1) encoding ADH protein sequence (SEQ ID NO: 2) was amplified from the OCT megaplasmid from Pseudomonas putida GPo1 using primers AlkJ-BamHI-F (ACTGGGATCCGATGTACGACTATATAATCGTTGGTGCTG; SEQ ID NO: 3) and AlkJ-BglII-R (ACTGAGATCTTTACATGCAGACAGCTATCATGGCC; SEQ ID NO: 4) and Phusion DNA polymerase (available from Thermo). The PCR product was double digested with BamHI and BglII, and then ligated to same digested pRSFDuet plasmid (available from Novagen) with T4 DNA ligase. The ligation product was transformed (heat shock) into E. coli T7 Expression competent cells (available from New England Biolabs) to give pRSF-AlkJ. In the next step, the cvωTA gene nucleic acid sequence (SEQ ID NO: 5) encoding ωTA protein sequence (SEQ ID NO: 6) from Chromobacterium violaceum was first synthesized and codon optimized for E. coli according the published sequence. Using this synthesized DNA as template, the gene was amplified using primers CvTA-BglII-RBS-F (CAGATCTTAAGGAGATATATAATGCAAAAACAACGCACCACCTCAC; SEQ ID NO: 7) and CvTA-XhoI-EcoRI-R (ACTGCTCGAGGAATTCTTACGCCAGGCCACGAGCTTTCAG; SEQ ID NO: 8) and Phusion DNA polymerase. The PCR product was double digested with BglII and XhoI, and then ligated to pRSF-AlkJ (digested with BglII and XhoI) with T4 DNA ligase. The ligation product was transformed (heat shock) into E. coli T7 Expression competent cells to give pRSF-AlkJ-CvTA. On the other hand, aladh gene nucleic acid sequence (SEQ ID NO: 9) encoding AlaDH protein sequence (SEQ ID NO: 10) was amplified from the genome of Bacillus subtilis str.168 using primers AlaDH-EcoRI-RBS-F (ACTGGAATTCTAAGGAGATATATAATGATCATAGGGGTTCCTAAAGAGAT; SEQ ID NO: 11) and AlaDH-XhoI-R (ACTGCTCGAGTTAAGCACCCGCCACAGATGATTCA; SEQ ID NO: 12). The PCR product was double digested with EcoRI and XhoI, and then ligated to pRSF-AlkJ-CvTA (digested with EcoRI and XhoI). The ligation product was transformed (heat shock) into E. coli T7 Expression competent cells to give Module 3 on pRSFDuet-1 plasmid, namely R-M3. Similarly, Module 3 was also sub-cloned to other three vectors by the following procedures. Module 3 operon was amplified with primers AlkJ-BamHI-F (ACTGGGATCCGATGTACGACTATATAATCGTTGGTGCTG; SEQ ID NO: 13) and AlaDH-XhoI-R (ACTGCTCGAGTTAAGCACCCGCCACAGATGATTCA; SEQ ID NO: 14), digested with BamHI and XhoI, and then ligated to double digested pACYCDuet-1, pCDFDuet-1, and pETDuet-1 (available from Novagen). The transformation of these products gave A-M3, C-M3, and E-M3, respectively.

### Example 2

### Production of (S)-Phenylethanol Amine from (S)-Phenylethane Diol Via Cascade Biocatalysis by Using E. coli Containing Module 3 and Expressing AlkJ, CvωTA, and AlaDH

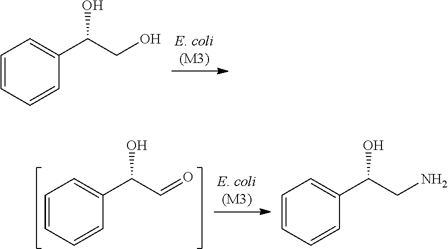

The recombinant E. coli strain containing the plasmid pRSF-AlkJ-CvTA-AlaDH (R-M3) was grown in 1 mL LB medium containing kanamycin (50 mg/L) at 37° C. and with 2% of the culture inoculated into 25 mL M9 medium containing glucose (20 g/L), yeast extract (6 g/L), and kanamycin (50 mg/L). When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in KPB buffer (200 mM, pH 8.0) to 10 g cdw/L and used for biotransformation of (S)-phenylethane diol (40 mM) with 0.5% glucose (for cofactor regeneration) and NH₃—NH₄Cl (200 mM, pH 8.25). The reaction was conducted at 30° C. and 300 rpm in a 100-mL flask for 24 h. Aliquots of the aqueous sample (100 μL) were taken during the reaction and analyzed by reverse phase HPLC (Agilent poroshell 120 EC-C18 column, acetonitrile:water (0.1% TFA)=30:70, and flow rate of 0.5 mL/min) to quantify the production of amino alcohol. (S)-phenylethanol amine was produced from (S)-phenylethane diol, with the highest conversion of 80% yield (32 mM) obtained in 4 h (FIG. 11b). This result showed that the constructed recombinant strain is a powerful catalyst for the cascade biotransformation of (S)-phenylethane diol to (S)-phenylethanol amine.

### Example 3

### Genetic Engineering of E. coli Containing Module 4 and Expressing HMO, EclIvE, GluDH, and KatE

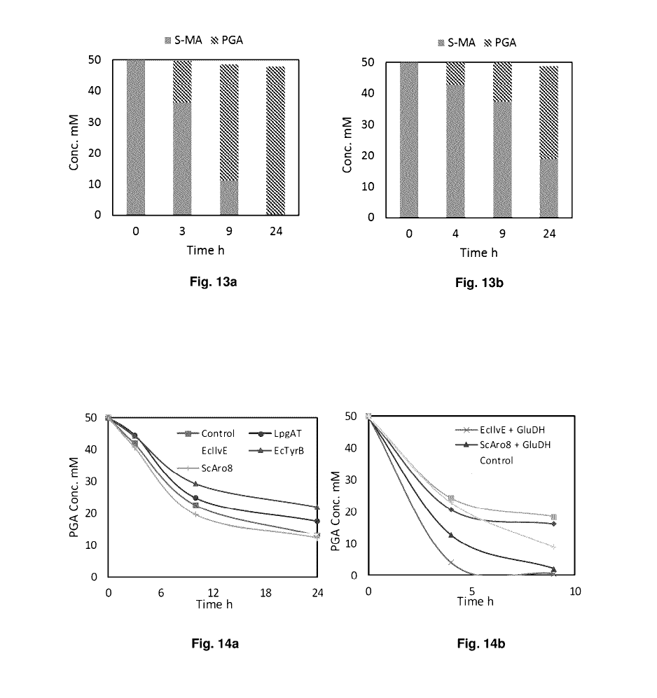

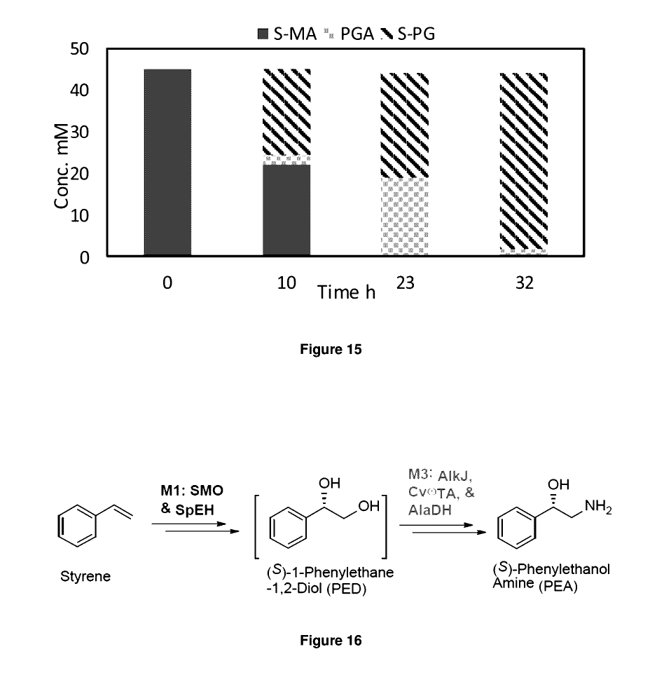

Briefly, Module 4 requires a cascade transformation of (S)-mandelic acid to (S)-phenylglycine via oxidation and reductive amination (FIG. 12). Two different enzymes were known to oxidize (S)-mandelic acid to phenylglyoxylic acid: (S)-mandelate dehydrogenase (MDH) in the mandelic acid degradation pathway of Pseudomonas putida ATCC 12633, and hydroxymandelate oxidase (HMO) in the vancomycin biosynthesis pathway of Streptomyces coelicolor A3(2). Both enzymes were successfully cloned and overexpressed in E. coli, and the corresponding whole cells were evaluated for oxidation of 50 mM (S)-mandelic acid to phenylglyoxylic acid (FIGS. 13a and 13b). Clearly, HMO showed higher activity and fully converted 50 mM substrates in 24 h. Thus, HMO was chosen for the Module 4. The second reaction in Module 4 is enantioselective amination of phenylglyoxylic acid to L-phenylglycine. We focused on α-transaminase (α-TA) for amination of phenylglyoxylic acid. Four different α-TAs were cloned and tested: L-phenylglycine transaminase (LpgAT) from Streptomyces pristinaespiralis branch chain amino acid transaminase (IlvE) from E. coli, aromatic amino acid transaminase (TyrB) from E. coli, and aromatic amino acid transaminase (Aro8) from Saccharomyces cerevisiae. The E. coli cells expressing these α-TAs were examined for amination of 50 mM phenylglyoxylic acid with 200 mM glutamate as amino donor. As shown in FIG. 14a, phenylglyoxylic acid was converted, though incompletely, and the best two are EclIvE and ScAro8. Obviously, the transamination is a reversible reaction and difficult to complete in this reaction. The glutamate dehydrogenase (GluDH) from E. coli was cloned to regenerate glutamate (direct amine donor) using ammonia. GluDH was successfully expressed alone and together with EclIvE or ScAro8. The corresponding E. coli strains were evaluated for amination of 50 mM phenylglyoxylic acid with 200 mM ammonia (FIG. 14b). E. co/i co-expressing EclIvE and GluDH was found to be the best biocatalyst for complete amination of the phenylglyoxylic acid in 9 h. To construct Module 4, we first combined HMO, EclIvE, and GluDH together as an operon. Since HMO is a H₂O₂ generating oxidase, a catalase, KatE, from E. coli, was further integrated to improve the oxidation of phenylglyoxylic acid of HMO. With that, Module 4 containing four enzymes was constructed (FIG. 12), and the four enzymes were clearly co-expressed in E. coli containing Module 4 (FIG. 8, lane M4). Cascade transformation was optimized to convert 45 mM (S)-mandelic acid to 42.5 mM (S)-phenylglycine (95% yield) by 32 h (FIG. 15). Similarly, the Module 4 was also sub-cloned to four plasmids to give A-M4, C-M4, E-M4, and R-M4, respectively.

More particularly, the hmo gene nucleic acid sequence (SEQ ID NO: 15) encoding HMO protein sequence (SEQ ID NO: 16) was first synthesized and codon optimized for E. coli according to the published sequence (Li, T. L. et al. Chem. Commun. 2001, 1752-1753). Using this synthesized DNA as template, the gene was amplified using primers HMO-BspHI-F (ACTGTCATGATGCGTGAACCGCTGACGCTGGATG; SEQ ID NO: 17) and HMO-EcoRI-R (ACTGGAATTCTTAGCCGTGAGAACGATCGCGATGC; SEQ ID NO: 18). The PCR product was double digested with BspHI and EcoRI, and then ligated to pRSF (digested with NcoI and EcoRI) with T4 DNA ligase. The ligation product was transformed (heat shock) into E. coli T7 Expression competent cells to give pRSF-HMO. Next, ilvE gene nucleic acid sequence (SEQ ID NO: 19) encoding α-TA protein sequence (SEQ ID NO: 20) was amplified from the genome of E. coli K12 MG 1655 using primers IlvE-EcoRI-RBS-F (ACTGGAATTC TAAGGAGATATATAATGACCACGAAGAAAGCTGATTACA; SEQ ID NO: 21) and IlvE-BglII-R (ACTGAGATCTTTATTGATTAACTTGATCTAACCAGCCC; SEQ ID NO: 22). The PCR product was digested with EcoRI and BglII, ligated to pRSF-HMO (digested with EcoRI and BglII), and then transformed (heat shock) into E. coli T7 Expression competent cells to yield pRSF-HMO-IlvE. Similarly, gludh gene nucleic acid sequence (SEQ ID NO: 23) encoding GluDH protein sequence (SEQ ID NO: 24) was amplified from the genome of E. coli K12 MG 1655 using primers GluDH-BglII-RBS-F (ACTGAGATCTTAAGGAGATATATAATGGATCAGACATATTCTCTGGAGTC; SEQ ID NO: 25) and GluDH-KpnI-R (ACTGGTACCTTAAATCACACCCTGCGCCAGCATC; SEQ ID NO: 26). The PCR product was digested with BglII and KpnI, ligated to pRSF-HMO-IlvE (digested with BglII and KpnI), and then transformed into E. coli competent cells to offer pRSF-HMO-IlvE-GluDH. In the last step, catalase gene katE nucleic acid sequence (SEQ ID NO: 27) encoding CAT protein sequence (SEQ ID NO: 28) was amplified from the genome of E. coli K12 MG 1655 using primers KatE-KpnI-RBS-F (ACTGGGTACCTAAGGAGATATATAATGTCGCAACATAACGAAAAGAACC; SEQ ID NO: 29) and KatE-XhoI-R (ACTGCTCGAGTCAGGCAGGAATTTTGTCAATCTTAG; SEQ ID NO: 30). The PCR product was digested with KpnI and XhoI, ligated to pRSF-HMO-IlvE-GluDH (digested with KpnI and XhoI), and then transformed into E. coli competent cells to offer pRSF-HMO-IlvE-GluDH-KatE (Module 4 on pRSF, R-M4). Similarly, Module 4 was also sub-cloned to other three vectors by first amplified with primers HMO-BspHI-F (ACTGTCATGATGCGTGAACCGCTGACGCTGGATG; SEQ ID NO: 31) and KatE-XhoI-R (ACTGCTCGAGTCAGGCAGGAATTTTGTCAATCTTAG; SEQ ID NO: 32), digested with BspHI and XhoI, and then ligated to double digested pACYCduet, pCDFduet, and pETduet. The transformation of these products gave A-M4, C-M4, and E-M4, respectively.

### Example 4

### Production of (S)-Phenylglycine from (S)-Mandelic Acid Via Cascade Biocatalysis by Using E. coli Containing Module 4 and Expressing HMO, EclIvE, GluDH, and KatE

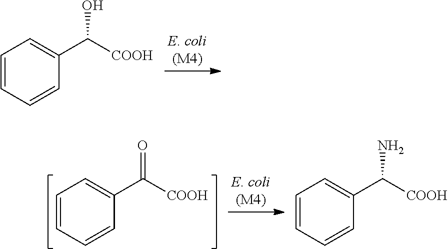

The recombinant E. coli strain containing the plasmid pRSF-HMO-EclIvE-GluDH-KatE (R-M4) was grown in 1 mL LB medium containing kanamycin (50 mg/L) at 37° C. and with 2% of the culture inoculated into 25 mL M9 medium containing glucose (20 g/L), yeast extract (6 g/L), and kanamycin (50 mg/L). When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in KPB buffer (200 mM, pH 8.0) to 10 g cdw/L and used for biotransformation of (S)-mandelic acid (45 mM) with 0.5% glucose (for cofactor regeneration) and 50 mM NH₃—NH₄Cl (pH 8.25). The reaction was conducted at 30° C. and 300 rpm in a 100-mL flask for 32 h. At 23 h, additional 1% glucose (for cofactor regeneration) and NH₃—NH₄Cl (100 mM, pH 8.25) were added to complete the reaction. Aliquots of the aqueous sample (100 μL) were taken during the reaction and analysed by reverse phase HPLC (Agilent poroshell 120 EC-C18 column, acetonitrile:water (0.1% TFA)=30:70, and flow rate of 0.5 mL/min) to quantify the production of phenylglycine. (S)-Phenylglycine was produced from (S)-mandelic acid, with the highest conversion at 95% yield (42.5 mM) obtained in 32 h (FIG. 15). This result showed that the constructed recombinant strain is a powerful catalyst for the cascade biotransformation of (S)-mandelic acid to (S)-phenylglycine.

### Example 5

### Genetic Engineering of E. coli Containing Module 1 and Module 3, Coexpressing SMO, SpEH, AlkJ, CvωTA, and AlaDH

To achieve formal asymmetric aminohydroxylation of styrene to chiral (S)-phenylethanol amine (FIG. 16), Module 1 (formal dihydroxylation) and Module 3 (alcohol to amine) need to be combined together.

Module 1 (M1) is as described in WO 2014189469 and comprises styrene monooxygenase gene styAB with nucleic acid sequence (SEQ ID NOs: 33 and 35) encoding SMO protein sequences (SEQ ID NO: 34 and 36) and an spEH gene with nucleic acid sequence (SEQ ID NO: 37) encoding epoxide hydrolase protein sequence (SEQ ID NO: 38). Four E. coli strains containing A-M3, C-M3, E-M3, and R-M3 were grown in 1 mL LB media containing appropriate antibiotic (50 mg/L chloramphenicol, 50 mg/L streptomycin, 100 mg/L ampicillin, or 50 mg/L kanamycin) at 37° C. overnight. The culture (100 μL) was then inoculated into 5 mL of fresh LB media containing appropriate antibiotic at 37° C. until OD₆₀₀ reached 0.5 (about 2 h). The cells were then harvested by centrifugation (2500 g, 10 min, 4° C.) and resuspended in 1 mL of cold CaCl₂ solution (0.1 M) on ice. The cell suspension was kept on ice and shaken at 90 rpm for 2 h, and then harvested by centrifugation (2500 g, 8 min, 4° C.) and resuspended in 0.2-0.5 mL of cold CaCl₂ solution (0.1 M) to obtain the competent cells of E. coli containing A-M3, C-M3, E-M3, and R-M3, respectively. Next, 0.5 μL of plasmid A-M1, C-M1, E-M1, and R-M1 (100-500 ng/μL) was transformed into the competent cells according to standard heat shock procedure (on ice for 30 min, 42° C. for 90 sec, ice for 5 min, recovery at 37° C. for 45 min). The recombinant cells were spread on LB agar plates with two appropriate antibiotics. The twelve combinatorial transformations gave twelve E. coli strains AC, AE, AR, CA, CE, CR, EA, EC, ER, RA, RC, RE. Each of these twelve strains contains both Module 1 and Module 3, and expresses five enzymes (SMO, SpEH, AlkJ, CvωTA, and AlaDH).

### Example 6

### Production of (S)-Phenylethanol Amine from Styrene Via Cascade Biocatalysis by Using E. coli Containing Module 1 and Module 3

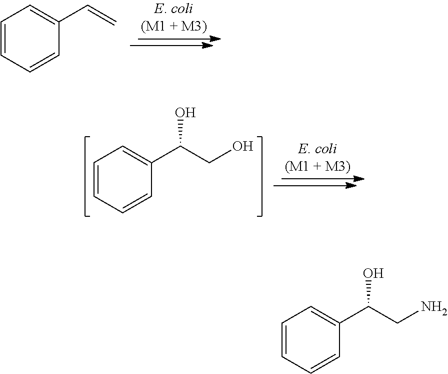

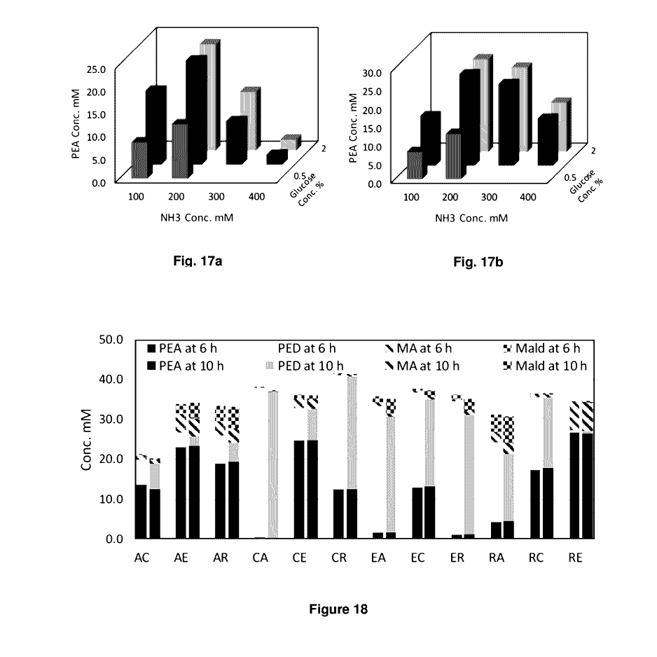

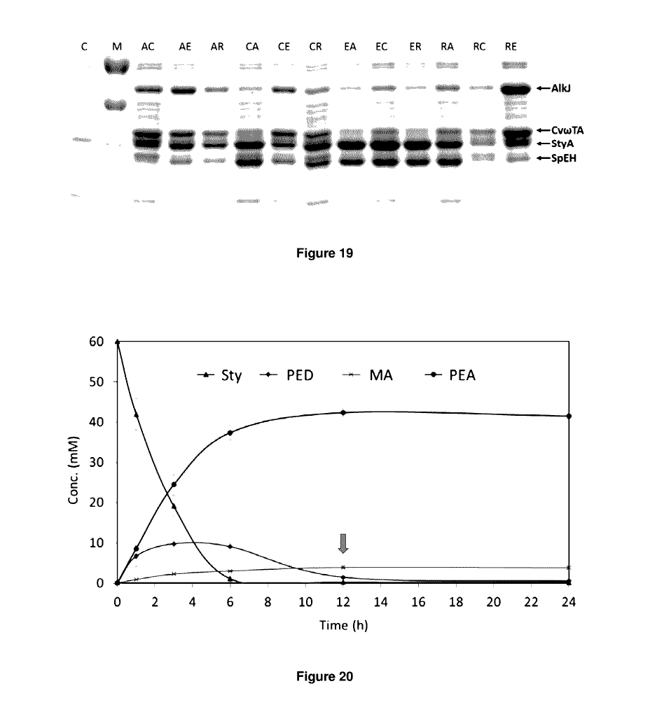

Since the reaction is complex, the cascade reaction was first optimized by applying different amount of glucose and ammonia. Biotransformation of 50 mM styrene was performed with resting cells of E. coli strain AR and CE (two representative strains co-expressing five enzymes) with 0.5-2% glucose and 100-400 mM ammonia. The result indicated that the best condition is 2% glucose with 200 mM ammonia for both strains (FIGS. 17a and 17b). The twelve recombinant E. coli strains containing M1 and M3 were grown in 1 mL LB medium containing two appropriate antibiotics at 37° C. and then with 2% inoculated into 25 mL M9 medium containing glucose (20 g/L), yeast extract (6 g/L), and two appropriate antibiotics. When OD₆₀₀ reached 0.6, IPTG (0.5 mM) was added to induce the expression of enzymes. The cells continued to grow and express enzymes for 12 hours at 22° C. before they were harvested by centrifugation (2500 g, 10 min). The cells were resuspended in KPB buffer (200 mM, pH 8.0) to 10 g cdw/L with 2% glucose (for cofactor regeneration) and NH₃—NH₄Cl (200 mM, pH 8.25). A 2 mL n-hexadecane containing styrene (50 mM) was added to the reaction system to form a second phase. The reaction was conducted at 30° C. and 300 rpm in a 100-mL flask for 24 h. Aliquots of the aqueous sample (100 μL) were taken during the reaction and analyzed by reverse phase HPLC (Agilent poroshell 120 EC-C18 column, acetonitrile:water (0.1% TFA)=30:70, and flow rate of 0.5 mL/min) to quantify the production of amino alcohol. Using the optimal condition, all the twelve E. coli strains were individually evaluated for cascade transformation of 50 mM styrene, resulting in very different amount of (S)-phenylethanol amine in 10 h (FIG. 18). (S)-phenylethanol amine was produced from styrene, with high conversion of 44-54% yield (22-27 mM) obtained in 6 hours and, more importantly, in excellent e.e., using the best three strains AE, CE, RE (FIG. 18). This result demonstrated the feasibility of the cascade biotransformation of alkenes (styrene) to chiral amino alcohols ((S)-phenylethanol amine). A SDS-PAGE analysis was also performed to examine the expression of five enzymes (SMO, SpEH, AlkJ, CvωTA, and AlaDH) (FIG. 19). Strong expression of AlkJ and CvωTA (Module 3) was observed for the best three strains (AE, CE, RE) while strong expression of SMO and SpEH (Module 1) often led to substantial accumulation of intermediate phenylethane diol. The current system can be further improved with optimization of reaction conditions or using more efficient enzymes.

### Example 7

### Efficient Production of (S)-Phenylethanol Amine from Styrene Via Cascade Biocatalysis by Using E. coli (AE)

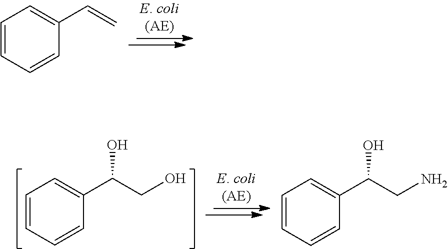

The best E. coli strain containing M1 and M3, E. coli (AE), was grown in 1 mL LB medium containing two appropriate antibiotics at 37° C. and with 2% of the culture inoculated into 25 mL M9 medium containing glucose (20 g/L), yeast extract (6 g/L), and two appropriate antibiotics. When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in 200 mM of NaP buffer (Na₂HPO₄—NaH₂PO₄, pH 8.0) to 15 g cdw/L with 2% glucose and NH₃—NH₄Cl (200 mM, pH 8.25). A 2 mL n-hexadecane containing styrene (60 mM) was added to the reaction system to form a second phase. The reaction was conducted at 25° C. and 300 rpm in a 100-mL flask for 24 h. Additional 0.5% glucose and NH₃/NH₄Cl (100 mM) were added into the system at 12 h. Aliquot of the aqueous sample (100 μL) were taken during the reaction and analyzed by reverse phase HPLC (Agilent poroshell 120 EC-C18 column, acetonitrile:water (0.1% TFA)=30:70, and flow rate of 0.5 mL/min) to quantify the production of amino alcohol. The conversion of styrene to (S)-phenylethanol amine was achieved in 70% yield (42 mM) and 98% e.e. in 12 h (FIG. 20). It is worth noting that unreacted substrates, intermediates, and by-products were kept in low amounts 5 mM). More importantly, (R)-phenylethanol amine (phenylglycinol) was not observed, indicating the high stereo-selectivity of the cascade biotransformation. The result demonstrated the potential of the cascade biotransformation for further scaling up and optimization.

### Example 8

### Efficient Production of Substituted (S)-Phenylethanol Amines from Substituted Styrenes Via Cascade Biocatalysis by Using E. coli (AE)

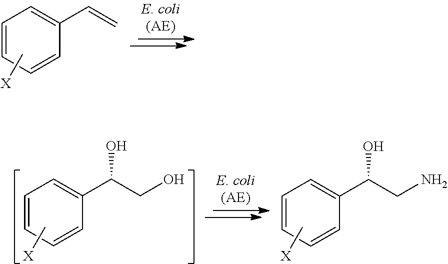

E. coli (AE) was grown in 1 mL LB medium containing two appropriate antibiotics at 37° C. and with 2% of the culture inoculated into 25 mL M9 medium containing glucose (20 g/L), yeast extract (6 g/L), and two appropriate antibiotics. When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in 200 mM of NaP buffer (Na₂HPO₄—NaH₂PO₄, pH 8.0) to 10 g cdw/L with 1% glucose and NH₃—NH₄Cl (200 mM, pH 8.25). A 2 mL n-hexadecane containing substituted styrene (20 mM) was added to the reaction system to form a second phase. The reaction was conducted at 25° C. and 300 rpm in a 100-mL flask for 24 h. Additional 0.5% glucose and NH₃/NH₄Cl (100 mM) were added in to the system at 12 h. An aliquot of the aqueous sample (100 μL) was taken at the end of reaction and analysed by reverse phase HPLC (Agilent poroshell 120 EC-C18 column, acetonitrile:water (0.1% TFA)=30:70, and flow rate of 0.5 mL/min) to quantify the production of amino alcohol. The enantiomeric excess (e.e.) of amino alcohols was determined by chiral HPLC analysis (Daicel Crownpak CR(+) column, mobile phase 100-90% water with 0.1% TFA and 0-10% methanol). As shown in Table 2, all the eleven (substituted) (S)-phenylethanol amines were produced in good to excellent e.e. of 91% and moderate to good yield in 24 h, demonstrating the relative broad scope of the cascade biotransformation.

TABLE 2
Regio- and enantioselective one-pot conversion of substituted styrenes
1a-k to substituted (S)-phenylethanol amines 6a-k with E. coli (AE).

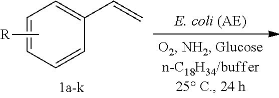

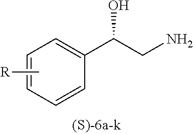

| | | | Yield | e.e. |
|---|---|---|---|---|
| Substrate* | R group | Product | (%)^{†} | (%)^{‡} |
| 1a | H | (S)-6a | 86 | 98 |
| 1b | o-F | (S)-6b | 65 | >99 |
| 1c | m-F | (S)-6c | 71 | 97 |
| 1d | p-F | (S)-6d | 78 | 91 |
| 1e | m-Cl | (S)-6e | 20 | 99 |
| 1f | p-Cl | (S)-6f | 36 | >99 |
| 1g | m-Br | (S)-6g | 16 | 99 |
| 1h | p-Br | (S)-6h | 26 | 96 |
| 1i | m-Me | (S)-6i | 81 | >99 |
| 1j | p-Me | (S)-6j | 69 | 96 |
| 1k | m-OMe | (S)-6k | 81 | 98 |

### Example 9

### Genetic Engineering of E. coli Containing Module 1, Module 2, and Module 4, Coexpressing SMO, SpEH, AlkJ, EcALDH, HMO, EclIvE, GluDH, and CAT

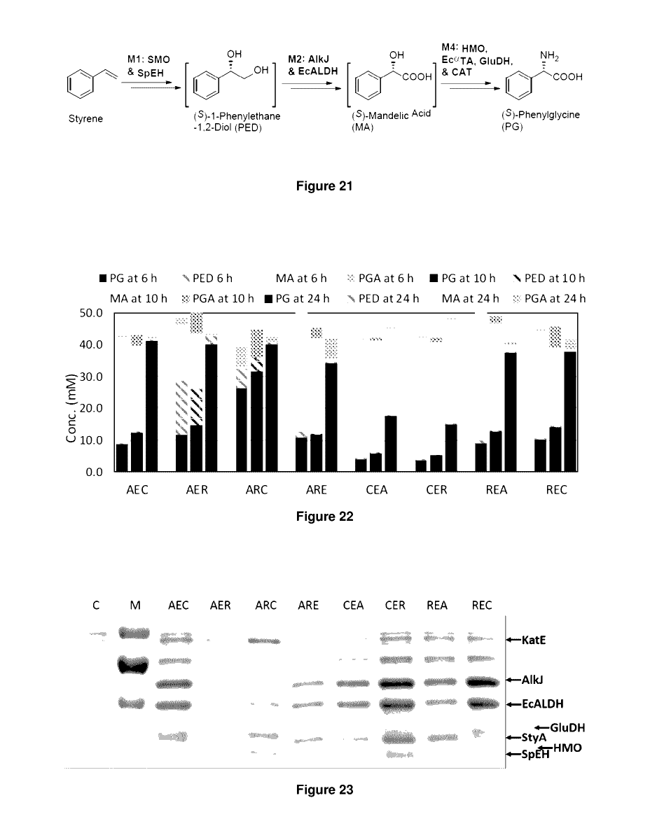

To achieve more challenging asymmetric oxy- and amino-functionalization of alkene to chiral α-amino acid, three modular transformations need to be combined together, including Module 1 (dihydroxylation), Module 2 (terminal oxidation), and Module 4 (sub-terminal amination) (FIG. 21). Module 2 (M2) is as described supra and comprises alcohol dehydrogenase gene alkJ with nucleic acid sequence (SEQ ID NO: 1) encoding ADH protein sequence (SEQ ID NO: 2) and an aldehyde dehydrogenase gene aldh with nucleic acid sequence (SEQ ID NO: 39) encoding EcALDH protein sequence (SEQ ID NO: 40) amplified using F primer CGAGATCTTAAGGAGATATATAATGACAGAGCCGCATGTAGCAGTATTA (SEQ ID NO: 58) and R primer ACTGCTCGAGTTAATACCGTACACACACCGACTTAG (SEQ ID NO: 59). The following four E. coli strains each containing both Module 1 and Module 2 were engineered by co-transforming two plasmids in E. coli T7 Expression competent cells: E. coli (A-M1_E-M2), E. coli (A-M1_R-M2), E. coli (C-M1_E-M2), E. coli (R-M1_E-M2). These four strains served as parental strains for further integration with Module 4. The four E. coli strains were grown in 1 mL LB media containing two appropriate antibiotics (50 mg/L chloramphenicol, 50 mg/L streptomycin, 100 mg/L ampicillin, or 50 mg/L kanamycin) at 37° C. overnight. The culture (100 μL) was then inoculated into 5 mL fresh LB media containing two appropriate antibiotics at 37° C. until OD₆₀₀ reached 0.5 (about 2 h). The cells were then harvested by centrifugation (2500 g, 10 min, 4° C.) and resuspended in 1 mL cold CaCl₂ solution (0.1 M) on ice. The cell suspension was kept on ice and shaken at 90 rpm for 2 h, and then harvested by centrifugation (2500 g, 8 min, 4° C.) and resuspended in 0.2-0.5 mL cold CaCl₂ solution (0.1 M) to obtain the competent cells of E. coli strains. Next, 0.5 μL of plasmid A-M4, C-M4, E-M4, and R-M4 (100-500 ng/μL) was transformed into the competent cells according to standard heat shock procedure (on ice for 30 min, 42° C. for 90 sec, ice for 5 min, recovery at 37° C. for 45 min). The recombinant cells were spread on LB agar plates with three appropriate antibiotics. The eight combinatorial transformations gave eight E. coli strains AEC, AER, ARC, ARE, CEA, CER, REA, REC, each containing Module 1, Module 2, and Module 4 on different plasmids, and expressing eight enzymes, SMO, SpEH, AlkJ, EcALDH, HMO, EclIvE, GluDH, and CAT.

### Example 10

### Production of (S)-Phenylglycine from Styrene Via Cascade Biocatalysis by Using E. coli Containing Module 1, Module 2, and Module 4

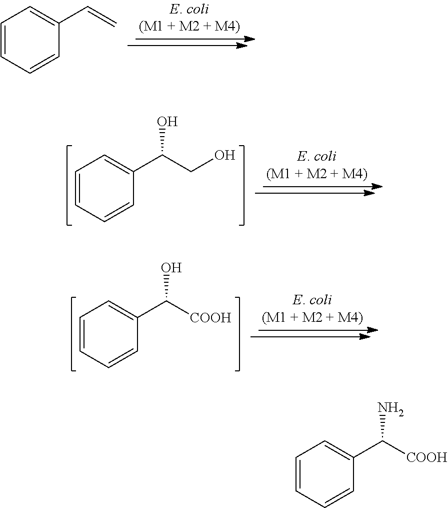

The eight recombinant E. coli strains containing M1, M2, and M4 were grown in 1 mL LB medium containing three appropriate antibiotics at 37° C. and with 2% of the culture inoculated into 25 mL M9 medium containing glucose (20 g/L), yeast extract (6 g/L), and three appropriate antibiotics. When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in KPB buffer (200 mM, pH 8.0) to 10 g cdw/L with 0.5% glucose (for cofactor regeneration) and NH₃—NH₄Cl (50 mM, pH 8.25). A 2 mL n-hexadecane containing styrene (50 mM) was added to the reaction system to form a second phase. The reaction was conducted at 30° C. and 300 rpm in a 100-mL flask for 24 h. At 20 h, additional 1% glucose (for cofactor regeneration) and NH₃—NH₄Cl (100 mM, pH 8.25) were added to complete the reaction. Aliquots of the aqueous sample (100 μL) were taken during the reaction and analyzed by reverse phase HPLC (Agilent poroshell 120 EC-C18 column, acetonitrile:water (0.1% TFA)=30:70, and flow rate of 0.5 mL/min) to quantify the production of amino acid. (S)-phenylglycine was produced from styrene with the highest conversion at 80% (40 mM) obtained in 24 h using the best strain ARC (FIG. 22). Other intermediates including phenylethane diol, mandelic acid, and phenylglyoxylic acid, only accounted for less than 5 mM of the total concentrations. This is a significant achievement that incorporated six selective enzymatic transformations that allow the conversion of initial substrates to products in more than 80% yield. This clearly demonstrated the feasibility of the synthetic route from alkene (styrene) to chiral α-amino acid ((S)-phenylglycine). A SDS-PAGE analysis was performed for the whole-cell protein of all the eight strains (FIG. 23).

### Example 11

### Efficient Production of (S)-Phenylglycine from Styrene Via Cascade Biocatalysis by Using E. coli (ARC)

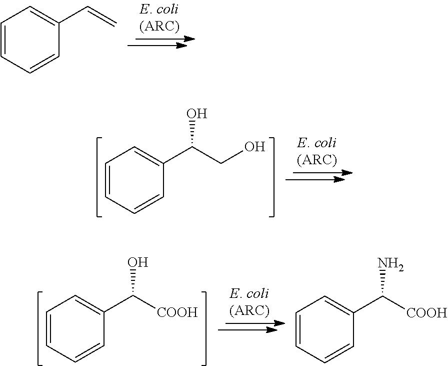

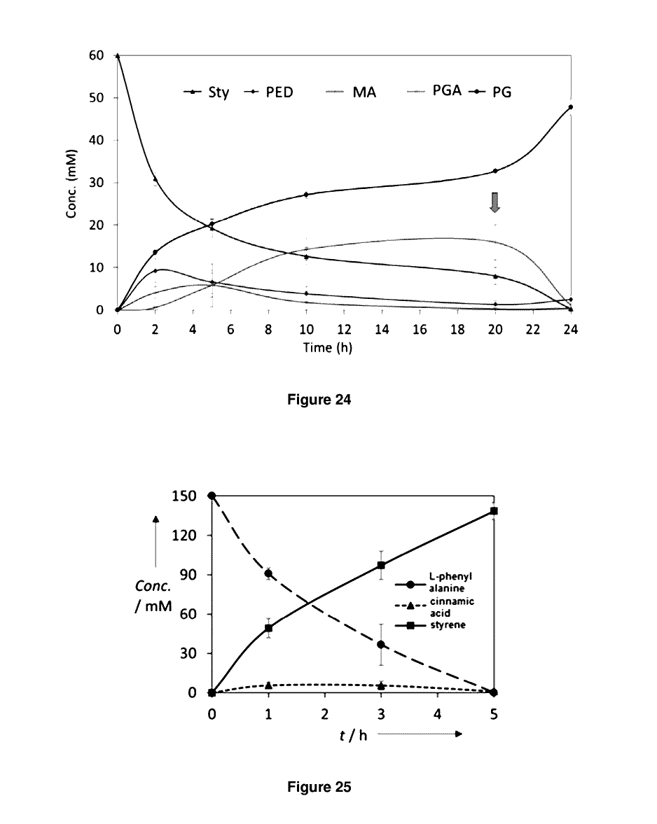

The best E. coli strain containing M1, M2, and M4, E. coli (ARC), was grown in 1 mL LB medium containing two appropriate antibiotics at 37° C. and with 2% of the culture inoculated into 25 mL M9 medium containing glucose (20 g/L), yeast extract (6 g/L), and two appropriate antibiotics. When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in KP buffer (200 mM, pH 8.0) to 15 g cdw/L with 0.5% glucose and NH₃—NH₄Cl (100 mM, pH 8.25). A 2 mL n-hexadecane containing styrene (60 mM) was added to the reaction system to form a second phase. The reaction was conducted at 25° C. and 300 rpm in a 100-mL flask for 24 h. Additional 0.5% glucose and NH₃/NH₄Cl (100 mM) were added into the system at 20 h. Aliquots of the aqueous sample (100 μL) were taken during the reaction and analyzed by reverse phase HPLC (Agilent poroshell 120 EC-C18 column, acetonitrile:water (0.1% TFA)=30:70, and flow rate of 0.5 mL/min) to quantify the production of amino acid. The conversion of styrene to (S)-phenylglycine was achieved in 80% yield (48 mM) and in 99% e.e. in 24 h (FIG. 24). It is worth noting that unreacted substrates, intermediates, and by-products were kept in low amounts 5 mM). The result demonstrated the potential of the cascade biotransformation for further scaling up and optimization.

### Example 12

### Efficient Production of Substituted (S)-Phenylglycines from Substituted Styrenes Via Cascade Biocatalysis by Using E. coli (ARC)

E. coli (ARC), was grown in 1 mL LB medium containing two appropriate antibiotics at 37° C. and with 2% of the culture inoculated into 25 mL M9 medium containing glucose (20 g/L), yeast extract (6 g/L), and two appropriate antibiotics. When OD₆₀₀ reached 0.6, IPTG (0.5 mM) was added to induce the expression of enzymes. The cells continued to grow and express enzymes for 12 h at 22° C. before they were harvested by centrifugation (2500 g, 10 min). The cells were resuspended in KP buffer (200 mM, pH 8.0) to 10 g cdw/L with 0.5% glucose and NH₃—NH₄Cl (100 mM, pH 8.25). A 2 mL n-hexadecane containing substituted styrene (20 mM) was added to the reaction system to form a second phase. The reaction was conducted at 25° C. and 300 rpm in a 100-mL flask for 24 h. Additional 0.5% glucose and NH₃/NH₄Cl (100 mM) were added into the system at 20 h. An aliquot of the aqueous sample (100 μL) was taken at the end of reaction and analysed by reverse phase HPLC (Agilent poroshell 120 EC-C18 column, acetonitrile:water (0.1% TFA)=30:70, and flow rate of 0.5 mL/min) to quantify the production of amino acids. The enantiomeric excess (e.e.) of amino acids was determined by chiral HPLC analysis (Daicel Crownpak CR(+) column, mobile phase 100-90% water with 0.1% TFA and 0-10% methanol). As shown in Table 3, all the eleven (substituted) (S)-phenylglycines were produced in excellent e.e. of 90% and moderate to good yield in 24 h. This demonstrated the relative broad scope of the cascade biotransformation, with potential to extent to other amino acids.

TABLE 3
Regio- and enantioselective one-pot conversion of substituted styrenes
1a-k to (S)-phenylglycines 8a-k with E. coli (ARC).

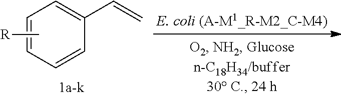

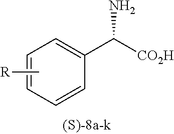

| | | | Yield | e.e. |
|---|---|---|---|---|
| Substrate* | R group | Product | (%)^{†} | (%)^{‡} |
| 1a | H | (S)-8a | 88 | >99 |
| 1b | o-F | (S)-8b | 55 | 99 |
| 1c | m-F | (S)-8c | 91 | >99 |
| 1d | p-F | (S)-8d | 76 | >99 |
| 1e | m-Cl | (S)-8e | 73 | >99 |
| 1f | p-Cl | (S)-8f | 63 | >99 |
| 1g | m-Br | (S)-8g | 60 | >99 |
| 1h | p-Br | (S)-8h | 28 | >99 |
| 1i | m-Me | (S)-8i | 73 | >99 |
| 1j | p-Me | (S)-8j | 63 | >99 |
| 1k | m-OMe | (S)-8k | 68 | >99 |

### Example 13

### Genetic Engineering of Plasmids Containing PAD-PAL

The synthesized gene of AnFDC (fdc1) with nucleic acid sequence (SEQ ID NO: 41) encoding AnPAD protein sequence (SEQ ID NO: 42) was amplified using primers “ACTGTCATGAGCGCGCAACCTGCGCACCTG” (SEQ ID NO: 43) and “ACTGGAATTCTTAGTTACTGAAGCCCATTTTGGTC” (SEQ ID NO: 44) with Phusion DNA polymerase. The PCR product was double-digested with BspHI and EcoRI, and then ligated to the NcoI and EcoRI digested pRSFDuet-1 with T4 DNA ligase. The ligation product was transformed into E. coli T7 Expression competent cells to give pRSF-AnFDC. On the other hand, the synthesized gene of AnPAD (pad1) with nucleic acid sequence (SEQ ID NO: 45) encoding AnPAD protein sequence (SEQ ID NO: 46) was amplified using primers “ACTGGAATTCTAAGGAGATATATCATGTTCAACTCACTTCTGTCCGGC” (SEQ ID NO: 47) and “ACTGCTGCAGTTATTTTTCCCAACCATTCCAACG” (SEQ ID NO: 48). The PCR product was double digested with EcoRI and PstI, and then ligated to the same digested pRSF-AnFDC with T4 DNA ligase. The ligation product was transformed into E. coli T7 Expression competent cells to give pRSF-PAD plasmid. Then, the gene of AtPAL2 with nucleic acid sequence (SEQ ID NO: 49) encoding PAL protein sequence (SEQ ID NO: 50) was amplified from the cDNA library of Arabidopsis thaliana (purchased from ATCC 77500) using primers “ACTGCATATGGATCAAATCGAAGCAATGTTGTG” (SEQ ID NO: 51) and “ACTGCTCGAGTTATTTTTCCCAACCATTCCAACG” (SEQ ID NO: 52). The PCR product was double digested with NdeI and XhoI, and then ligated to the same digested pRSF-PAD with T4 DNA ligase. The ligation product was transformed into E. coli T7 Expression competent cells to give pRSF-PAD-PAL plasmid. PAD-PAL was also sub-cloned to the other three vectors by the following procedure. PAD-PAL was amplified with primers “ACTGTCATGAGCGCGCAACCTGCGCACCTG” (SEQ ID NO: 43) and “ACTGCTCGAGTTATTTTTCCCAACCATTCCAACG” (SEQ ID NO: 52), digested with BspHI and XhoI, and then ligated to NcoI and XhoI digested pACYCDuet-1, pCDFDuet-1, and pETDuet-1. The transformation of these products gave pACYC-PAD-PAL, pCDF-PAD-PAL, and pET-PAD-PAL, respectively.

### Example 14

### Production of Styrene from L-Phenylalanine Via Cascade Biocatalysis Using E. coli Strain (pRSF-PAL-PAD)

The recombinant E. coli strain (pRSF-PAL-PAD) was first inoculated in 1 mL LB medium containing appropriate antibiotics (50 mg/L chloramphenicol, 50 mg/L streptomycin, 100 mg/L ampicillin, 50 mg/L kanamycin or a combination of them) at 37° C. for 7-10 h. The culture was then transferred into 25 mL M9 medium containing glucose (20 g/L), yeast extract (6 g/L) and appropriate antibiotics in a 125 mL tri-baffled flask. The cells were grown at 37° C. and 300 r.p.m. for about 2 h to reach an OD₆₀₀ of 0.6, followed by the addition of IPTG (0.5 mM) to induce the enzyme expression. The cells were grown for 12-13 h at 22° C. to reach late exponential phase, and they were collected by centrifugation (3500 g, 10 min). The cell pellets were resuspended to a cell density of 15 gcdw/L in KP buffer (200 mM, pH 8) containing glucose (0.5%, w/v) and substrate L-phenylalanine (150 mM). Next, 2 mL of above cell suspension and 2 mL n-hexadecane were added into a shaking flask (100 mL). The reaction mixture was incubated in a shaking incubator at 250 rpm and 30° C. for 5 h. Aliquots of each phase (100 μL) were taken out at 1 h, 3 h, and 5 h during the course of the reaction. For organic phase, 50 μL of n-hexadecane was separated after centrifugation (13000 g, 2 min), diluted with 950 μL n-hexane (containing 2 mM benzyl alcohol as internal standard), and analyzed by GC-FID for quantifying styrene. For aqueous phase, 20 μL of supernatant was separated after centrifugation (13000 g, 2 min), diluted with 480 μL TFA solution (0.5%) and 500 μL acetonitrile (containing 2 mM benzyl alcohol as internal standard), and then analysed by reverse phase HPLC for L-phenylalanine and cinnamic acid. E. coli (pRSF-PAL-PAD) efficiently converted 150 mM of L-phenylalanine into 139 mM of styrene in 92% yield at 5 h with no accumulation of the intermediate by-products (FIG. 25), demonstrating the potential of using amino acids as the starting material for the synthesis of other valuable compounds.

### Example 15

### Genetic Engineering of Plasmids Containing SMO

The styA gene of SMO was amplified from pSPZ10 (Panke, S., et al., Biotechnology and bioengineering, 2000, 69(1): 91-100) using primers “ACTGTCATGAAAAAGCGTATCGGTATTGTTGG” (SEQ ID NO: 53) and “ACTGGAATTCTCATGCTGCGATAGTTGGTGCGAACTG” (SEQ ID NO: 54) with Phusion DNA polymerase. The PCR product was double-digested with BspHI and EcoRI, and then ligated to the NcoI and EcoRI digested pRSFDuet-1 with T4 DNA ligase. The ligation product was transformed into E. coli T7 Expression competent cells to give pRSF-StyA. On the other hand, the styB gene of SMO was amplified from pSPZ10 using primers “ACTG GAATTCTAAGGAGATTTCAAATGACGCTGAAAAAAGATATGGC” (SEQ ID NO: 55) and “ACTGGGTACCTCAATTCAGTGGCAACGGGTTGC” (SEQ ID NO: 56). The PCR product was double digested with EcoRI and KpnI, and then ligated to the same digested pRSF-StyA with T4 DNA ligase. The ligation product was transformed into E. coli T7 Expression competent cells to give pRSF-SMO plasmid. SMO was also sub-cloned to the other three vectors by the following procedure. SMO was amplified with primers “ACTGTCATGAAAAAGCGTATCGGTATTGTTGG” (SEQ ID NO: 53) and “ACTGGGTACCTCAATTCAGTGGCAACGGGTTGC” (SEQ ID NO: 56), digested with BspHI and KpnI, and then ligated to NcoI and KpnI digested pACYCDuet-1, pCDFDuet-1, and pETDuet-1. The transformation of these products gave pACYC-SMO, pCDF-SMO, and pET-SMO, respectively.

Genetic engineering of plasmids containing SMO-StEH: The previously constructed SST1 plasmid (S. Wu, et al., ACS Catal. 2014, 4: 409) was used as pRSF-SMO-StEH plasmid in this study. SMO was also sub-cloned to the other three vectors by the following procedure. SMO-StEH was amplified with primers “ACTGTCATGAAAAAGCGTATCGGTATTGTTGG” (SEQ ID NO: 53) and “ACTGCTCGAGTTAGAATTTTTGAATAAAATC” (SEQ ID NO: 57), digested with BspHI and XhoI, and then ligated to NcoI and XhoI digested pACYCDuet-1, pCDFDuet-1, and pETDuet-1. The transformation of these products gave pACYC-SMO-StEH, pCDF-SMO-StEH, and pET-SMO-StEH, respectively.

The 4 plasmids containing SMO-SpEH (pACYC-SMO-SpEH, pCDF-SMO-SpEH, pET-SMO-SpEH, and pRSF-SMO-SpEH) had been engineered in a previous project (S. Wu, et al., Nat. Commun. 2016, 7: 11917) thus they were directly used in this study.

The 4 plasmids containing AlkJ-EcALDH (pACYC-AlkJ-EcALDH, pCDF-AlkJ-EcALDH, pET-AlkJ-EcALDH, and pRSF-AlkJ-EcALDH) had been engineered in a previous project (S. Wu, et al., Nat. Commun. 2016, 7: 11917) thus they were directly used in this study.

The 4 plasmids containing HMO-EcaTA-GluDH-CAT (pACYC-HMO-EcaTA-GluDH-CAT, pCDF-HMO-EcaTA-GluDH-CAT, pET-HMO-EcaTA-GluDH-CAT, and pRSF-HMO-EcaTA-GluDH-CAT) had been engineered in a previous project (S. Wu, et al., Nat. Commun. 2016, 7: 11917) thus they were directly used in this study.

### Example 16

### Engineering of E. coli Strains Harboring Multiple Plasmids

The full list of strains and the plasmids contained is provided in Table 1.

Engineering of E. coli (LZ01-12): each of PAD-PAL plasmids (pACYC-PAD-PAL, pCDF-PAD-PAL, pET-PAD-PAL, and pRSF-PAD-PAL) and each of SMO plasmids (pACYC-SMO, pCDF-SMO, pET-SMO, and pRSF-SMO) were co-transformed into E. coli T7 Expression competent cells to give E. coli (LZ01-12).

Engineering of E. coli (LZ13-24): each of PAD-PAL plasmids (pACYC-PAD-PAL, pCDF-PAD-PAL, pET-PAD-PAL, and pRSF-PAD-PAL) and each of SMO-StEH plasmids (pACYC-SMO-StEH, pCDF-SMO-StEH, pET-SMO-StEH, and pRSF-SMO-StEH) were co-transformed into E. coli T7 Expression competent cells to give E. coli (LZ13-24).

Engineering of E. coli (LZ25-36): each of PAD-PAL plasmids (pACYC-PAD-PAL, pCDF-PAD-PAL, pET-PAD-PAL, and pRSF-PAD-PAL) and each of SMO-SpEH plasmids (pACYC-SMO-SpEH, pCDF-SMO-SpEH, pET-SMO-SpEH, and pRSF-SMO-SpEH) were co-transformed into E. coli T7 Expression competent cells to give E. coli (LZ25-36).

Engineering of the competent cells of E. coli (LZ25-36): E. coli (LZ25-36) strains were inoculated in 1 mL LB medium containing appropriate antibiotics at 37° C. overnight. 100 μL overnight culture was inoculated into 5 mL fresh LB medium containing appropriate antibiotics and grew at 37° C. until OD₆₀₀ reached 0.5 (about 2 h). The cells were harvested by centrifugation (2500 g, 10 min, 4° C.) and resuspended with 1 mL cold CaCl₂ solution (0.1 M) on ice. The cell suspension was kept on ice and shaken at 90 rpm for 2 h, and then harvested by centrifugation (2500 g, 8 min, 4° C.) and resuspended in 0.2-0.5 mL cold CaCl₂ solution (0.1 M) to obtain the competent cells of E. coli.

Engineering of E. coli (LZ37-60): the AlkJ-EcALDH plasmids (pACYC-AlkJ-EcALDH, pCDF-AlkJ-EcALDH, pET-AlkJ-EcALDH, and pRSF-AlkJ-EcALDH) were transformed into E. coli (LZ25-36) competent cells to give E. coli (LZ37-60).

Engineering of E. coli (LZ61-84): each of the AlkJ-EcALDH plasmids (pACYC-AlkJ-EcALDH, pCDF-AlkJ-EcALDH, pET-AlkJ-EcALDH, and pRSF-AlkJ-EcALDH) and each of the HMO-EcaTA-GluDH-CAT plasmids (pACYC-HMO-EcaTA-GluDH-CAT, pCDF-HMO-EcaTA-GluDH-CAT, pET-HMO-EcaTA-GluDH-CAT, and pRSF-HMO-EcaTA-GluDH-CAT) were co-transformed into E. coli (LZ25-36) competent cells to give E. coli (LZ61-84).

### Example 17

### Biotransformation of (S)-1 to (S)-4 with E. coli (LZ01-L12)

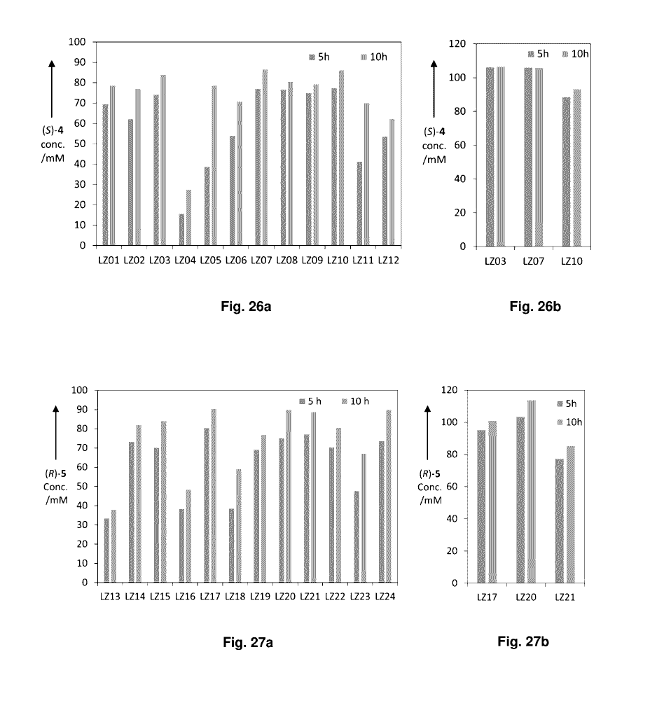

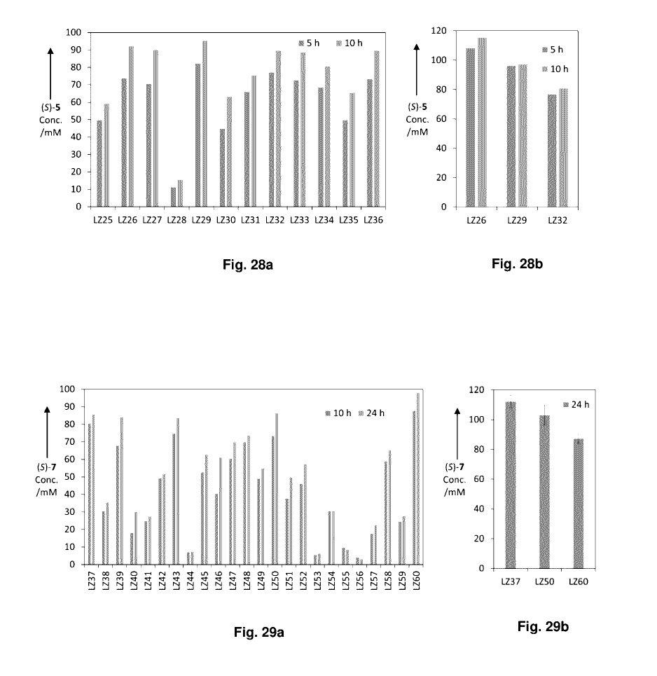

Freshly prepared E. coli (LZ01-L12) cells were resuspended to a cell density of 10-15 g cdw L⁻¹ in KP buffer (200 mM, pH 8) containing glucose (2%, w/v) and substrate L-phenylalanine (S)-1 (100-140 mM). 2 mL of above cell suspension and 2 mL n-hexadecane was added into a shaking flask (100 mL). The reaction mixture was incubated in a shaking incubator at 250 rpm and 30° C. for 10 h. 100 μL aliquots of each phase were taken out at 1 h, 3 h, 5 h, and 10 h for following the reaction. For organic phase, 50 μL of n-hexadecane was separated after centrifugation (13000 g, 2 min), diluted with 950 μL n-hexane (containing 2 mM benzyl alcohol as internal standard), and analyzed by GC-FID for quantifying styrene 3 and (S)-styrene oxide 4. For aqueous phase, 20 μL of supernatant was separated after centrifugation (13000 g, 2 min), diluted with 480 μL TFA solution (0.5%) and 500 μL acetonitrile (containing 2 mM benzyl alcohol as internal standard), and then analyzed by reverse phase HPLC for L-phenylalanine (S)-1, cinnamic acid 2, and diol 5 (generated from autohydrolysis of 4). At the end of the reaction, the organic phase was separated by centrifugation (13000 g, 2 min) and used to determine the e.e. of 4 by chiral HPLC analysis. FIG. 26a shows biotransformation of 100 mM L-phenylalanine (S)-1 to (S)-styrene oxide 4 with 12 different E. coli strains (10 g cdw L⁻¹ resting cells) coexpressing PAL, PAD, and SMO. FIG. 26b shows biotransformation of 120 mM (S)-1 to (S)-4 with the best three E. coli strains (15 g cdw L⁻¹ resting cells). The reaction was performed in a two-phase system containing KP buffer (200 mM, pH 8, 2% glucose) and n-hexadecane (1:1) and shaken at 250 rpm and 30° C.

### Example 18

### Preparation of (S)-4 from Biotransformation of (S)-1 with E. coli (LZ03)

Newly prepared E. coli (LZ03) cells were suspended in KP buffer (200 mM, pH 8) containing glucose (2%, w/v) to form a 100 mL suspension (20 g cdw L⁻¹) in a tri-baffled flask (1 L). 1.652 g of (S)-1 (solid) and 100 mL of n-hexadecane was added into the flask to start the reaction at 250 rpm and 30° C. 100 μL aliquots of the aqueous and organic phases were taken out at different time points to follow the reaction. After 24 h, the reaction mixture was subjected to centrifugation (4000 g, 15 min) to remove the cells and the aqueous phase. The n-hexadecane phase was collected and extracted with acetonitrile two times (2×100 mL), and the acetonitrile phase was collected and dried over Na₂SO₄. After filtration, the acetonitrile phase was subjected to evaporation by using a rotary evaporator (Buchi Rotavapor® R-215) to remove the solvent. The crude (S)-4 product was purified by flash chromatography on a silica gel column with EtOAc:n-hexane of 1:50 as eluent (R₁≈0.3). The collected fractions were subjected to GC-FID analysis to confirm the purity. The organic solvent of the desired fractions was removed by evaporation, and the product was dried overnight under vacuum.

(S)-Styrene oxide 4 was obtained as colorless oil: 926 mg, 77% yield from 1, 99% ee, [α]_{D}²⁰: +24° (c 1.0, CHCl₃) {literature^{[34]} [α]_{D}²³: +23.6° (c 0.83, CHCl₃), 95% e.e.}. ¹H NMR (400 MHz, CDCl₃): δ=7.30-7.17 (m, 5H), 3.79-3.77 (dd, J=4.0, 2.8 Hz, 1H), 3.08-3.05 (dd, J=5.6, 4.0 Hz, 1H), 2.74-2.71 (dd, J=4.0, 2.8 Hz, 1H) ppm; ¹³C NMR (100 MHz, CDCl₃): δ=136.6, 127.5, 127.5, 127.2, 124.5, 124.5, 51.3, 50.2 ppm.

### Example 19

### Biotransformation of (S)-1 to (R)- or (S)-5 with E. coli (LZ13-LZ36)

Freshly prepared E. coli (LZ13-L36) cells were resuspended to a cell density of 10-15 g cdw L⁻¹ in KP buffer (200 mM, pH 8) containing glucose (2%, w/v) and substrate L-phenylalanine (S)-1 (100-120 mM). 2 mL of above cell suspension and 2 mL n-hexadecane was added into a shaking flask (100 mL). The reaction mixture was incubated in a shaking incubator at 250 rpm and 30° C. for 10 h. 100 μL aliquots of each phase were taken out at 1 h, 3 h, 5 h, and 10 h for following the reaction. For organic phase, 50 μL of n-hexadecane was separated after centrifugation (13000 g, 2 min), diluted with 950 Lit n-hexane (containing 2 mM benzyl alcohol as internal standard), and analyzed by GC-FID for quantifying styrene 3 and (S)-styrene oxide 4. For aqueous phase, 20 μL of supernatant was separated after centrifugation (13000 g, 2 min), diluted with 480 μL TFA solution (0.5%) and 500 μL acetonitrile (containing 2 mM benzyl alcohol as internal standard), and then analyzed by reverse phase HPLC for L-phenylalanine (S)-1, cinnamic acid 2, and product diol 5. At the end of the reaction, the aqueous phase was separated by centrifugation (13000 g, 2 min) and used to determine the e.e. of 5 by chiral HPLC analysis. FIG. 27a shows biotransformation of 100 mM L-phenylalanine (S)-1 to (R)-1-phenylethane-1,2-diol 5 with twelve different E. coli strains (10 g cdw L⁻¹ resting cells) coexpressing PAL, PAD, SMO, and StEH. FIG. 27b shows biotransformation of 120 mM (S)-1 to (R)-5 with the best three E. coli strains (15 g cdw L⁻¹ resting cells). The reaction was performed in a two-phase system containing KP buffer (200 mM, pH 8, 2% glucose) and n-hexadecane (1:1) and shaken at 250 rpm and 30° C.

### Example 20

### Preparation of (R)- or (S)-5 from Biotransformation of (S)-1 with E. coli (LZ20) or E. coli (LZ26)

Newly prepared E. coli (LZ20) or E. coli (LZ26) cells were suspended in KP buffer (200 mM, pH 8) containing glucose (2%, w/v) to form a 100 mL suspension (20 g cdw L⁻¹) in a tri-baffled flask (500 mL). 1.652 g of (S)-1 (solid) and 10 mL of ethyl oleate was added into the flask to start the reaction at 250 rpm and 30° C. 100 μL aliquots of the aqueous phases were taken out at different time points to follow the reaction. After 24 h, the reaction mixture was subjected to centrifugation (4000 g, 15 min) to remove the cells and the organic phase. The aqueous phase was collected, saturated with NaCl, and extracted with ethyl acetate (3×100 mL). The organic phase was collected and dried over Na₂SO₄. After filtration, the organic phase was subjected to evaporation by using a rotary evaporator to remove the solvent. The crude product was purified by flash chromatography on a silica gel column with EtOAc:n-hexane of 1:1 as eluent (R_{f}≈0.3). The collected fractions were subjected to GC-FID analysis to confirm the purity. The organic solvent of the desired fractions was removed by evaporation, and the product was dried overnight under vacuum.

(R)-1-Phenylethane-1,2-diol 5 was obtained as white solid: 975 mg, 71% yield from 1, 96% e.e., [α]_{D}²⁰: −37° (c 1.0, EtOH) {literature^{[35]} [α]_{D}²⁵: −37.8° (c 1.0, EtOH), 99% e.e.}. ¹H NMR (400 MHz, CDCl₃): δ=7.35-7.28 (m, 5H), 4.80-4.77 (dd, J=8.4, 3.2 Hz, 1H), 3.74-3.70 (m, 1H), 3.65-3.60 (m, 1H) ppm; ¹³C NMR (100 MHz, CDCl₃): δ=140.7, 128.7, 128.7, 128.2, 126.3, 126.3, 74.9, 68.2 ppm.

(S)-1-Phenylethane-1,2-diol 5 was obtained as white solid: 1082 mg, 78% yield from 1, 97% ee, [α]_{D}²⁰: +37° (c 1.0, EtOH) {literature^{[36]} [α]_{D}²³: +38.4° (c 4.38, EtOH), 99% e.e.}. ¹H NMR (400 MHz, CDCl₃): δ=7.34-7.25 (m, 5H), 4.79-4.75 (dd, J=8.4, 3.2 Hz, 1H), 3.73-3.69 (m, 1H), 3.64-3.59 (m, 1H) ppm; ¹³C NMR (100 MHz, CDCl₃): δ=140.7, 128.7, 128.7, 128.1, 126.3, 126.3, 74.9, 68.2 ppm. FIG. 28a shows biotransformation of 100 mM L-phenylalanine (S)-1 to (S)-1-phenylethane-1,2-diol 5 with 12 different E. coli strains (10 g cdw L⁻¹ resting cells) co-expressing PAL, PAD, SMO, and SpEH. FIG. 28b shows biotransformation of 120 mM (S)-1 to (S)-5 with the best three E. coli strains (15 g cdw L⁻¹ resting cells). The reaction was performed in a two-phase system containing KP buffer (200 mM, pH 8, 2% glucose) and n-hexadecane (1:1) and shaken at 250 rpm and 30° C.

### Example 21

### Biotransformation of (S)-1 to (S)-7 with E. coli (LZ37-LZ60)

Freshly prepared E. coli (LZ37-LZ60) cells were resuspended to a cell density of 10-15 g cdw L⁻¹ in KP buffer (200 mM, pH 8) containing glucose (0.5%, w/v) and substrate L-phenylalanine (S)-1 (100-120 mM). 2 mL of above cell suspension and 2 mL n-hexadecane was added into a shaking flask (100 mL). The reaction mixture was incubated in a shaking incubator at 250 rpm and 30° C. for 24 h. 100 μL aliquots of each phase were taken out at 1 h, 3 h, 5 h, 10 h, and 24 h for following the reaction. For organic phase, 50 μL of n-hexadecane was separated after centrifugation (13000 g, 2 min), diluted with 950 μL n-hexane (containing 2 mM benzyl alcohol as internal standard), and analyzed by GC-FID for quantifying styrene 3 and (S)-styrene oxide 4. For aqueous phase, 20 μL of supernatant was separated after centrifugation (13000 g, 2 min), diluted with 480 μL TFA solution (0.5%) and 500 μL acetonitrile (containing 2 mM benzyl alcohol as internal standard), and then analyzed by reverse phase HPLC for L-phenylalanine (S)-1, cinnamic acid 2, diol 5, and mandelic acid 7. At the end of reaction, aqueous phase was separated by centrifugation (13000 g, 2 min) and used to determine the e.e. of 7 by chiral HPLC analysis.

### Example 22

### Preparation of (S)-7 from biotransformation of (S)-1 with E. coli (LZ37)

Newly prepared E. coli (LZ37) cells were suspended in KP buffer (200 mM, pH 8) containing glucose (0.5%, w/v) to form a 100 mL suspension (20 g cdw L⁻¹) in a tri-baffled flask (500 mL). 1.652 g of (S)-1 (solid) and 10 mL of ethyl oleate was added into the flask to start the reaction at 250 rpm and 30° C. 100 μL aliquots of the aqueous phases were taken out at different time points to follow the reaction. After 24 h, the reaction mixture was subjected to centrifugation (4000 g, 15 min) to remove the cells and the organic phase. The aqueous phase was collected, saturated with NaCl, adjusted to pH=13 with NaOH (10 M), and washed with ethyl acetate two times (2×50 mL) to remove trace ethyl oleate and other organic impurities. The aqueous phase was adjusted to pH=1 with HCl (10 M) and extracted with ethyl acetate (3×100 mL). The organic phase was collected and dried over Na₂SO₄. After filtration, the organic phase was subjected to evaporation by using a rotary evaporator to remove the solvent. The crude product was purified by crystallization in ethyl acetate through dissolving at 65° C. and slowly cooling down to −20° C. The crystals were taken by filtration, and the mother liquor was evaporated and subjected to crystallization again. The collected crystals were combined and dried overnight under vacuum.

(S)-2-Hydroxy-2-phenylacetic acid 7 was obtained as white crystal: 1058 mg, 70% yield from 1, 99% e.e., [α]_{D}²⁰: +151° (c 1.0, H₂O) {literature^{[37]} [α]_{D}²⁰: +148.8° (c 0.5, H₂O), 99% ee}. ¹H NMR (400 MHz, D₂O): δ=7.36-7.30 (m, 5H), 5.18 (s, 1H) ppm; ¹³C NMR (100 MHz, D₂O): δ=176.1, 137.9, 129.0, 129.0, 127.0, 127.0, 72.9 ppm. FIG. 29a shows biotransformation of 100 mM L-phenylalanine (S)-1 to (S)-mandelic acid 7 with 24 different E. coli strains (10 g cdw L⁻¹ resting cells) co-expressing PAL, PAD, SMO, SpEH, AlkJ, and EcALDH. FIG. 29b shows biotransformation of 120 mM (S)-1 to (S)-7 with the best three E. coli strains (15 g cdw L⁻¹ resting cells). The reaction was performed in a two-phase system containing KP buffer (200 mM, pH 8, 0.5% glucose) and n-hexadecane (1:1) and shaken at 250 rpm and 30° C. The biotransformation in FIG. 29b was performed in triplicate, and error bars show ±s.d.

### Example 23

### Biotransformation of (S)-1 to (S)-9 with E. coli (LZ061-LZ84)

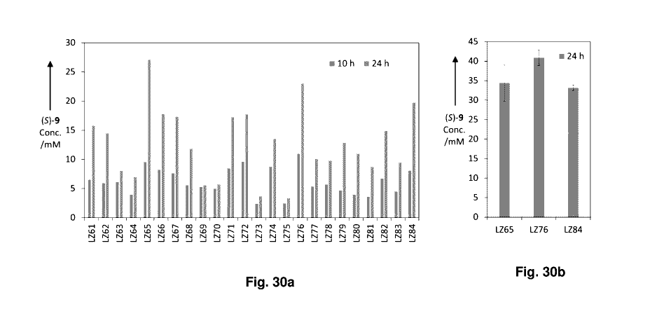

Freshly prepared E. coli (LZ61-LZ84) cells were resuspended to a cell density of 10-15 g cdw L⁻¹ in KP buffer (200 mM, pH 8) containing glucose (0.5%, w/v) and substrate L-phenylalanine (S)-1 (30-40 mM). 2 mL of above cell suspension and 2 mL n-hexadecane was added into a shaking flask (100 mL). The reaction mixture was incubated in a shaking incubator at 250 rpm and 30° C. for 24 h. 200 mM NH₃/NH₄Cl and 2% glucose was added at 10 h. 200 Lit aliquots of the mixture (100 μL of each phase) were taken out at 1 h, 3 h, 5 h, 10 h, and 24 h. 100 Lit HCl solution (0.8M) was mixed with 200 μL sample before centrifugation (13000 g, 2 min). For organic phase, 50 μL of n-hexadecane was separated and diluted with 950 μL n-hexane (containing 2 mM benzyl alcohol as internal standard), and analyzed by GC-FID for quantifying styrene 3 and (S)-styrene oxide 4. For aqueous phase, 100 μL of aqueous supernatant was separated and diluted with 400 μL TFA solution (0.1%) and 500 μL acetonitrile (containing 2 mM benzyl alcohol as internal standard), and then analyzed by reverse phase HPLC for L-phenylalanine (S)-1, cinnamic acid 2, diol 5, mandelic acid 7, keto acid 8, and phenylglycine 9. At the end of the reaction, the aqueous phase was separated by centrifugation (13000 g, 2 min) and used to determine the e.e. of 9 by chiral HPLC analysis. FIG. 30a shows biotransformation of 30 mM L-phenylalanine (S)-1 to (S)-phenylglycine 9 with 24 different E. coli strains (10 g cdw L⁻¹ resting cells) coexpressing PAL, PAD, SMO, SpEH, AlkJ, EcALDH, HMO, EcaTA, GluDH, and CAT. FIG. 30b shows biotransformation of 50 mM (S)-1 to (S)-9 with the best three E. coli strains (15 g cdw L⁻¹ resting cells). The reaction was performed in a two-phase system containing KP buffer (200 mM, pH 8, 0.5% glucose) and n-hexadecane (1:1) and shaken at 250 rpm and 30° C. 200 mM NH₃/NH₄Cl and 2% glucose was added at 10 h. The biotransformation in FIG. 30b was performed in triplicate, and error bars show ±s.d.

### Example 24

### Preparation of (S)-9 from Biotransformation of (S)-1 with E. coli (LZ76)

Newly prepared E. coli (LZ76) cells were suspended in KP buffer (200 mM, pH 8) containing glucose (0.5%, w/v) to form a 100 mL suspension (20 g cdw L⁻¹) in a tri-baffled flask (500 mL). 0.661 g of (S)-1 (solid) and 10 mL of ethyl oleate was added into the flask to start the reaction at 250 rpm and 30° C. 100 μL aliquots of the aqueous phases were taken out at different time points to follow the reaction. At 10 h, additional glucose (2%, w/v) and NH₃/NH₄Cl (200 mM) was added. After 24 h, the reaction mixture was first acidified with HCl (10 M) to pH=1 and subjected to centrifugation (4000 g, 15 min) to remove the cells and the organic phase. The collected aqueous phase was filtered to further remove solid impurities, followed by washing with ethyl acetate (2×50 mL) to remove trace ethyl oleate and other organic impurities. After neutralization to pH=7 with NaOH (10 M), the aqueous solution was concentrated to about 10 mL by evaporation to precipitate the amino acid. The solid was collected by filtration, washed with cold H₂O, and dried overnight under vacuum.

(S)-2-Amino-2-phenylacetic acid 9 was obtained as white solid: 376 mg, 62% yield from 1, >99% e.e., [α]_{D}²⁰: +149° (c 1.0, 1M HCl) {literature^{[38]} [α]_{D}²³: +150° (c 1.0, 1 M HCl), 99% e.e.}. ¹H NMR (400 MHz, D₂O containing 2% H₂SO₄): δ=7.34-7.29 (m, 5H), 5.02 (s, 1H) ppm; ¹³C NMR (100 MHz, D₂O containing 2% H₂SO₄): δ=170.6, 131.3, 130.3, 129.7, 129.7, 128.0, 128.0, 56.4 ppm.

### Example 25

### General Procedure for Fermentative Production of (S)-4, (R)-5, (S)-5, (S)-7, (S)-9

Recombinant E. coli strain (LZ03, LZ20, LZ26, LZ37, LZ76) was firstly inoculated in 1 mL LB medium containing appropriate antibiotic (50 mg L⁻¹ chloramphenicol, 50 mg L⁻¹ streptomycin, 100 mg L⁻¹ ampicillin, 50 mg L⁻¹ kanamycin, or a mixture of them) and grew at 37° C. for 7-10 h. The culture was inoculated into 50 mL M9 medium containing glucose (20 g L⁻¹), yeast extract (6 g L⁻¹), and appropriate antibiotics in a 250 mL tri-baffled flask. The cells continued to grow at 37° C. and 300 rpm for about 2 h to reach an OD₆₀₀ of 0.6, followed by the addition of IPTG to 0.5 mM to induce the enzyme expression. For the production of (S)-4 by E. coli (LZ03), 10 mL of ethyl oleate was added together with IPTG to reduce the autohydrolysis. The cells further grew at 22° C. for 12 h to reach late exponential phase. For the production of (R)-5, (S)-5, (S)-7, (S)-9, 1 mL aliquot of the medium was taken out and centrifuged (13000 g, 2 min). 500 μL supernatant was mixed with 500 μL acetonitrile (containing 2 mM benzyl alcohol as internal standard), and then analyzed by reverse phase HPLC for quantifying (R)-5, (S)-5, (S)-7, (S)-9. For production of (S)-4 by E. coli (LZ03), the whole culture (50 mL+10 mL ethyl oleate) was subjected to centrifugation (4000 rpm, 10 min). 500 μL ethyl oleate was taken and mixed with 500 μL n-hexane (containing 2 mM benzyl alcohol as internal standard) and analyzed by GC-FID for quantifying (S)-4.

1. A method for producing an enantiomerically pure or enantiomerically enriched 1,2-amino alcohol or α-amino acid, which method comprises subjecting an alkene starting material to multiple enzyme-catalyzed chemical transformations in a one-pot reaction system, wherein the method comprises generating a vicinal diol from the alkene and an α-hydroxyaldehyde from the vicinal diol.

2. The method of claim 1, wherein the method produces an enantiomerically pure or enantiomerically enriched 1,2-amino alcohol, comprising the steps of:
(a) generating a vicinal diol from an alkene by a dihydroxylation reaction catalyzed by a dioxygenase, or by conducting an epoxidation reaction catalyzed by an epoxidase to form an epoxide and conducting a hydrolysis reaction catalyzed by an epoxide hydrolase on the epoxide;
(b) generating an α-hydroxyaldehyde or a or an α-hydroxyketone from the vicinal diol by an oxidation reaction catalyzed by an alcohol oxidase or alcohol dehydrogenase; and
(c) generating a 1,2-aminoalcohol from the α-hydroxyaldehyde or α-hydroxyketone by a transamination reaction catalyzed by a transaminase or a reductive amination reaction catalyzed by an amine dehydrogenase.

3. The method of claim 2, wherein the method further comprises providing the alkene by generating a vinyl carboxylic acid from an α-amino acid by a deamination reaction catalyzed by an ammonia lyase and generating the alkene from the vinyl carboxylic acid in a decarboxylation reaction catalyzed by a decarboxylase.

4. The method of claim 2, wherein the alcohol oxidase is alditol oxidase or its mutants, or the alcohol dehydrogenase is selected from the group consisting of AlkJ from Pseudomonas putida, AlkJ homologue from Sphingomonas sp. HXN-200, dihydrodiol dehydrogenase, and mutants thereof.

5. The method of claim 2, wherein the transaminase is a ω-transaminase, or the amine dehydrogenase is a phenylalanine dehydrogenase, a leucine dehydrogenase or their mutants.

6. The method of claim 2, wherein the alkene has the formula (I): 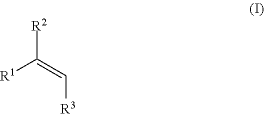
where:
R¹ to R³ independently represent H, an alkyl group, an alkenyl group, an alkynyl group, a cycloalkyl group, a cycloalkenyl group, an aryl group, an aryl alkyl group, a heterocyclic group, and a heterocyclic alkyl group, which groups are substituted or unsubstituted by one or more substituents, provided that at least one of R¹ to R³ is not H.

7. The method of claim 2, wherein the method comprises the use of one or more reactive components selected from the group consisting of cells, immobilized cells, cell extract, isolated enzymes and immobilized enzymes.

8. The method of claim 1, wherein the method produces an enantiomerically pure or enantiomerically enriched α-amino acid, comprising the steps of:
(a) generating a vicinal diol from an alkene by a dihydroxylation reaction catalyzed by a dioxygenase, or by conducting an epoxidation reaction catalyzed by an epoxidase to form an epoxide and conducting a hydrolysis reaction catalyzed by an epoxide hydrolase on the epoxide;
(b) generating an α-hydroxyaldehyde from the vicinal diol by an oxidation reaction catalyzed by an alcohol oxidase or alcohol dehydrogenase;
(c) generating an α-hydroxy acid from the α-hydroxyaldehyde in an oxidation reaction catalyzed by an aldehyde dehydrogenase or an aldehyde oxidase;
(d) generating an α-ketoacid from the α-hydroxy acid in an oxidation reaction catalyzed by a hydroxy acid dehydrogenase or a hydroxy acid oxidase; and
(e) generating an α-amino acid from the α-ketoacid by a transamination reaction catalyzed by a transaminase or a reductive amination reaction catalyzed by an amino acid dehydrogenase.

9. The method of claim 8, wherein the method further comprises providing the alkene by generating a vinyl carboxylic acid from an α-amino acid by a deamination reaction catalyzed by an ammonia lyase and generating the alkene from the vinyl carboxylic acid in a decarboxylation reaction catalyzed by a decarboxylase.

10. The method of claim 8, wherein the alcohol oxidase is alditol oxidase or its mutants, or the alcohol dehydrogenase is selected from the group consisting of AlkJ from Pseudomonas putida, AlkJ homologue from Sphingomonas sp. HXN-200, dihydrodiol dehydrogenase, and mutants thereof.

11. The method of claim 8, wherein the aldehyde dehydrogenase is AlkH from Pseudomonas putida or its mutants and/or phenyl aldehyde dehydrogenase from Escherichia coli or its mutants.

12. The method of claim 8, wherein the hydroxy acid dehydrogenase is mandelate dehydrogenase or its mutants, and the hydroxy acid oxidase is mandelate oxidase or its mutants and/or hydroxymandelate oxidase from S. coelicolor and its mutants.

13. The method of claim 8, wherein the transaminase is an α-transaminase.

14. The method of claim 8, wherein the alkene has the formula (II): 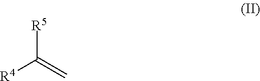
where:
R⁴ and R⁵ independently represent H, a straight chain or branched alkyl group, a straight chain or branched alkenyl group, a straight chain or branched alkynyl group, a cycloalkyl group, a cycloalkenyl group, an aryl group, an aryl alkyl group, and a heterocyclic group, which groups are substituted or unsubstituted by one or more substituents, provided that at least one of R⁴ and R⁵ is not H.

15. The method of claim 8, wherein the method comprises the use of one or more reactive components selected from the group consisting of cells, immobilized cells, cell extract, isolated enzymes and immobilized enzymes.

16. An isolated nucleic acid molecule encoding at least one heterologous catalytic enzyme selected from the group comprising:
(a) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(b) an alcohol oxidase or alcohol dehydrogenase for generating an α-hydroxyaldehyde or an α-hydroxyketone from the vicinal diol by an oxidation reaction; and
(c) a transaminase or an amine dehydrogenase for generating a 1,2-aminoalcohol from the α-hydroxyaldehyde or the α-hydroxyketone by a transamination reaction or a reductive amination; or
(d) a dioxygenase for generating a vicinal diol from an alkene by a dihydroxylation reaction, or an epoxidase for conducting an epoxidation reaction to form an epoxide and an epoxide hydrolase for conducting a hydrolysis reaction on the epoxide;
(e) an alcohol oxidase or alcohol dehydrogenase for generating an α-hydroxyaldehyde from the vicinal diol by an oxidation reaction;
(f) an aldehyde dehydrogenase or an aldehyde oxidase for generating an α-hydroxy acid from the α-hydroxyaldehyde in an oxidation reaction;
(g) a hydroxy acid dehydrogenase or a hydroxy acid oxidase for generating an α-ketoacid from the α-hydroxy acid in an oxidation reaction; and
(h) a transaminase or an amino acid dehydrogenase for generating an α-amino acid from the α-ketoacid by a transamination reaction or reductive amination reaction.

17. The isolated nucleic acid of claim 16, encoding a plurality of said catalytic enzymes.

18. The isolated nucleic acid molecule of claim 17, wherein said plurality of catalytic enzymes is arranged as at least one module selected from the group comprising:
i) a module comprising heterologous nucleic acid sequences that, when expressed, enzymatically transforms a terminal alkene to a 1,2-diol;
ii) a module comprising heterologous nucleic acid sequences that, when expressed, enzymatically transforms a 1,2-diol to a 1,2-amino alcohol;
iii) a module comprising heterologous nucleic acid sequences that, when expressed, enzymatically transforms a 1,2-diol to an α-hydroxy acid;
iv) a module comprising heterologous nucleic acid sequences that, when expressed, enzymatically transforms an α-hydroxy acid to an α-amino acid; and
v) a module comprising heterologous nucleic acid sequences that, when expressed, enzymatically transforms an L-amino acid to a terminal alkene,
or any combination thereof.

19. The isolated nucleic acid molecule of claim 18, comprising one or more modules selected from the group comprising:
i) a module comprising heterologous nucleic acid sequences that, when expressed, enzymatically transforms a terminal alkene to a 1,2-diol and a module comprising heterologous nucleic acid sequences that, when expressed, enzymatically transforms a 1,2-diol to a 1,2-amino alcohol;
ii) a module comprising heterologous nucleic acid sequences that, when expressed, enzymatically transforms a terminal alkene to a 1,2-diol and a module comprising heterologous nucleic acid sequences that, when expressed, enzymatically transforms a 1,2-diol to an α-hydroxy acid;
iii) a module comprising heterologous nucleic acid sequences that, when expressed, enzymatically transforms a terminal alkene to a 1,2-diol; a module comprising heterologous nucleic acid sequences that, when expressed, enzymatically transforms a 1,2-diol to a 1,2-amino alcohol and a module comprising heterologous nucleic acid sequences that, when expressed, enzymatically transforms an α-hydroxy acid to an α-amino acid;
optionally, a module comprising heterologous nucleic acid sequences that, when expressed, enzymatically transforms an L-amino acid to a terminal alkene,
or any combination thereof.

20. The isolated nucleic acid molecule of claim 18, wherein the isolated nucleic acid molecule encodes at least one enzyme selected from the group comprising:
i) at least one of SEQ ID NOs: 34, 36 and 38, variants, mutants, or fragments thereof to transform a terminal alkene to a 1,2-diol;
ii) at least one of SEQ ID NOs: 2 and 40, variants, mutants, or fragments thereof to transform a 1,2-diol to a 1,2-amino alcohol;
iii) at least one of SEQ ID NOs: 2, 6 and 10, variants, mutants, or fragments thereof to transform a 1,2-diol to an α-hydroxy acid;
iv) at least one of SEQ ID NOs: 16, 20, 24 and 28, variants, mutants, or fragments thereof to transform an α-hydroxy acid to an α-amino acid;
v) at least one of SEQ ID NOs: 42, 46 and 50, variants, mutants, or fragments thereof to transform an L-amino acid to a terminal alkene,
or any combination thereof.

21. The isolated nucleic acid molecule of claim 18, wherein the isolated nucleic acid molecule is selected from the group comprising:
i) at least one of SEQ ID NOs: 33, 35 and 37, variants, mutants, or fragments thereof to transform a terminal alkene to a 1,2-diol;
ii) at least one of SEQ ID NOs: 1 and 39, variants, mutants, or fragments thereof to transform a 1,2-diol to a 1,2-amino alcohol;
iii) at least one of SEQ ID NOs: 1, 5 and 9, variants, mutants, or fragments thereof to transform a 1,2-diol to an α-hydroxy acid;
iv) at least one of SEQ ID NOs: 15, 19, 23 and 27, variants, mutants, or fragments thereof to transform an α-hydroxy acid to an α-amino acid;
v) at least one of SEQ ID NOs: 41, 45 and 49, variants, mutants, or fragments thereof to transform an L-amino acid to a terminal alkene or any combination thereof.

22. The isolated nucleic acid molecule of claim 18, wherein the isolated nucleic acid molecule at least one enzyme selected from the group comprising:
i) SEQ ID NOs: 34, 36, 38, 2 and 40, variants, mutants, or fragments thereof to transform a terminal alkene to a 1,2-amino alcohol;
ii) SEQ ID NOs: 34, 36, 38, 2, 6 and 10, variants, mutants, or fragments thereof to transform a terminal alkene to an α-hydroxy acid;
iii) SEQ ID NOs: 34, 36, 38, 2, 40, 16, 20, 24 and 28 variants, mutants, or fragments thereof to transform a terminal alkene to an α-amino acid,
iv) SEQ ID NOs: 42, 46 and 50, variants, mutants, or fragments thereof to transform an L-amino acid to a terminal alkene,
or any combination thereof.

23. An expression construct comprising at least one nucleic acid molecule of claim 16.

24. One or more recombinant prokaryotic or eukaryotic cells selected from the group comprising bacterial cells, yeast cells, mammalian cells and insect cells, wherein said cells comprise at least one expression construct of claim 23.

25. The one or more prokaryotic or eukaryotic cells of claim 24, wherein said cells are recombinant bacterial cells.

26. The one or more recombinant cells of claim 24, wherein said enzymes have at least 60% amino acid identity with at least one enzyme selected from the group comprising an alcohol dehydrogenase with amino acid sequence represented by SEQ ID NO: 2, an ω-transaminase with amino acid sequence represented by SEQ ID NO: 5, an alanine dehydrogenase with amino acid sequence represented by SEQ ID NO: 9, a styrene monooxygenase with amino acid sequence represented by SEQ ID NOs: 34 & 36, an epoxide hydrolase with amino acid sequence represented by SEQ ID NO: 38, an aldehyde dehydrogenase with amino acid sequence represented by SEQ ID NO: 40; a phenylacrylic acid decarboxylase with amino acid sequence represented by SEQ ID NOs: 42 & 45 and a phenylalanine ammonia lyase with amino acid sequence represented by SEQ ID NO: 50.

27. The one or more recombinant cells of claim 24, wherein said cells express catalytic enzymes selected from the groups comprising;
i) styrene monooxygenase, epoxide hydrolase, alcohol dehydrogenase, ω-transaminase, and alanine dehydrogenase for producing a 1, 2 amino-alcohol from a terminal alkene; or
ii) styrene monooxygenase, epoxide hydrolase, alcohol dehydrogenase, aldehyde dehydrogenase, hydroxy acid oxidase, α-transaminase, glutamate dehydrogenase, and catalase, variants or bioactive fragments thereof for producing an alpha amino acid from a terminal alkene and,
optionally;
iii) lyase and decarboxylase, variants or bioactive fragments thereof, for producing a terminal alkene from an L-amino acid.

28. A kit comprising at least one isolated nucleic acid according to claim 16.
